SEQUENCE LISTING (27 sequences)

SEQ ID NO: 1 - DNA, 9587 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggattaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgactaggaagacttccgagcggtcgcagcctcgtggaaggcgacaacctatccccaaggctcgccagcccgagggcagggcctgggctcagcccggatacccttggcccctctacggcaatgagggtctggggtgggcaggatggctcctgtcaccccgtggctctcggcctagttggggccctacggacccccggcgtaggtcgcgtaatttgggtaaggtcatcgataccctcacatgcggcttcgccgacctcatggggtacatcccgctcgtcggcgcccccctaggaggcgctgccagggccctggcgcatggcgtccgggttctggaggacggcgtgaactatgcaacagggaatttgcccggttgctctttctctatcttcctcttggctttgctgtcctgtttgaccatcccagcttccgcttacgaagtgcgcaacgtatccggggtgtaccatgtcacgaacgactgctccaactcaagtattgtgtatgaggcagcggacatgatcatgcacacccccgggtgcgtgccctgcgttcgggaggacaattcctcccgttgctgggtagcgctcactcccacgcttgcggccaggaacagcagcgtccccaccacgacaatacgacgccacgtcgacttgctcgttggggcagctgctttctgttccgctatgtacgtgggagacctctgcggatctgttttcctcatttcccagctgttcaccttctcacctcgccggtatgagacggtgcaagactgcaactgctcgctctatcccggccacgtatcaggtcaccgcatggcttgggatatgatgatgaactggtcacctacaacagccctggtggtgtcgcagttgctccggatcccacaagccgtcgtggacatggtggcgggggcccactggggagtcctggcgggccttgcctactattccatggtggggaactgggctaaggtcttgattgtgatgctactttttgccggcgtcgacgggaacactcgtgtgtcagggggggaagcagccaagaacaccatgggctttgcatccctctttgtgtctggcccgtcgcagaaaatccaacttattaacaccaatggcagctggcacatcaacaggactgctctgaattgcgatgactccctccacaccgggttccttgctgcgctgttttacgcgcacaagttcaactcgtccggatgctcggggcgcatggccagctgccgccccattgacgagtttgcccaggggtggggtcccatcactcacggtgtgcctgacaacctggaccagaggccttactgctggcactacgcgcctcggccgtgcggtatcgtacccgcgtcgcaggtgtgtggcccagtgtattgcttcaccccgagccctgtcgtggtggggacgaccgatcgtttcggcgcccccacgtacagctggggggagaatgagacggacgtgctactccttaacaacacgcgaccgccacaaggcaactggttcggttgcacatggatgaacagcaccgggttcaccaagacgtgcgggggccccccgtgcaacattggaggggtcggcaacaacaccttgacctgtcctacggactgcttccggaagcaccccgaggccacttacaccaaatgcggctcgggcccttggttgacacctaggtgcatggttgactacccatacagactctggcactacccttgcactgtcaattttaccatcttcaaggtcaggatgtatgtagggggtgtggagcacaggctcgacgccgcgtgcaattggacccgaggagagcgttgcaatgtggaggacagggatagatcagagcttagcccactgctactgtccacaacagagtggcagatactgccctgttccttcaccaccctaccggctctgtccactggtttgatccacctccaccagaacatcgtggacgtgcaatacctgtacggtgtggggtcagtggttgtctccgttgtaatcagatgggagtatgtcgtgctgctcttccttctcctggcggacgcacgcgtctgcgcctgcttgtggatgatgctgctgatagctcaggctgaggccgccttagagaacttggtggtcctcaatgcggcatctgtagctggagcgcatggcactctctccttccttgtgttcttctgtgccgcctggtacatcaagggcaagctggtccctggagcggcatatgctttctatggcgtgtggccgctgctcctgctcctgctggcattaccaccacgagcatacgccatggaccgggagatggctgcatcatgcggaggcgcggtcttcataggtctggcactcctgaccttgtcaccacactataaagcgttcctcgctaggctcatttggtggttgcagtatcttatcaccagggtcgaggcgctactgcaagtgtgggtcccccctcttaacgttcgggggggccgcgatgccatcatcctcctcacgtgcgcggtccatccagagctaatctttgaaatcaccaaaatcttgctcgccataattggtccgctcatggtgctccaggctggcttaaccagagtgccgtacttcgtgcgcgctcaggggctcatccgtgtgtgcatgttggtgcggaaagtcgctgggggtcactacgtccagatggctctcatgaggctggccgcgctgacgggcacgtacgtttacaaccatcttactccgctgcgggactgggcccacgcgggcttgcgggaccttgtggtggcagttgagcccgtcgtcttctctgacatggagaccaagatcatcacctggggggcagacaccgcggcgtgtggggacatcatcttgggtctacccgtctccgcccgaagggggagggagatacttttgggaccggccgacagttatgaagggcagggatggcggcttcttgcgcctatcacggcctattcccaacagacgcggggcctactcggttgcatcatcactagtctcacaggccgggacaggaaccaggtcgagggggaggttcaggtggtttccaccgcaacgcagtccttcttggcgacctgcatcaatggtgtgtgttggactgtctaccatggtgccggctcaaagaccctagccggcccaaagggtccgatcacccaaatgtacaccaatgtagaccaggacctcgtcggctggcaggcgccccccggggcgcgttccttaacaccatgcacctgcggcagctcggacctctacttggtcacgaggcatgctgatgtcattccggtgcgccggcggggcgataacaggggcagcctgctttcccccagacctgtctcttacttaaagggctcctcgggtggtccactgctctgcccctcagggcacgctgtgggcatcttccgggccgccgtgtgcaccaggggggttgcgaaggcggtggactttgtgcccgttgagtccatggaaactaccatgcggtctccggtcttcacggacaattcatctcccccggccgtaccgcagacattccaagtggcccatctacacgctcccactggcagcggtaagagcactaaggtgccggctgcttatgcagcccaagggtacaaggtactcgtcctgaacccatccgttgccgccaccttaggttttggggcgtatatgtctaaagcacatggtgttgaccccaacatcagaactggggtaaggaccatcaccacaggcgctcccattacgtattccacttatggcaagttccttgccgacggtggttgctctgggggcgcctatgacatcataatatgtgatgagtgccactcaactgactcgacttccatcttgggcatcggcactgtcttggaccaagcggagacggctggagcgcggctcgtcgtgctcgccaccgctacacctccgggatcagtcaccgtgccacatcccaatatcgaggaggtggctttgtccaacactggagagatccccttttatggtaaggcctttcccatcgaggccatcaagggggggaggcatctcattttctgccactccaagaagaaatgtgacgagctcgccacaaagctgtcggcccttggaatcaacgctgtagcgtactaccggggtcttgatgtgtccgtcataccgacaagcggagatgtcgttgtcgtggcaacagacgctctgatgacgggctacactggcgactttgactcagtgatcgactgtaacacatgtgtcacccagacagtcgacttcagcttggaccctaccttcaccattgagacgacgaccgtgccccaagacgcggtatcgcgctcgcaacggcgaggcaggactggtaggggcagggggggcatatacaggtttgtgactccaggggaacggccctcgggcatgttcgattcttcggtcctgtgcgagtgctatgacgcgggctgtgcttggtacgagctcacgcccgccgagacctcggttaggttgcgggcttacctaaatacaccagggttgcccgtctgccaggaccatctggagttctgggagagcgtcttcacaggcctcacccacatagatgcccatttcttatcccagaccaagcaggcaggagacaacttcccctacctggtagcgtaccaggctacggtgtgcgccagggcccaggccccaccaccatcgtgggaccaaatgtggaagtgtctcatacggctaaagcctacactgcacgggccaacacccctgctgtacaggctaggagccgtccaaaacgaagtcactctcacacaccccataaccaaattcatcatggcatgcatgtcggctgacctagaggtcgtcacgagcacctgggtgctagtaggcggggtccttgcagctctggccgcgtactgcctgacaacgggcagcgtggtcattgtgggtaggatcatcttgtctgggaagccggctgtcattcccgatagggaagtgctctaccaggagttcgatgaaatggaagagtgcgcctcgcacctcccttacattgaacagggaatgcagctcgccgagcaattcaagcagaaggcgctcgggttgctgcaagcagccaccaagcaagcggaggctgctgctcccgtggtggagtccaagtggcaagcccttgaggccttttgggcgaagcacatgtggaatttcatcagcgggatacagtacttagcaggcttgtccactctgcctgggaaccccccgatagcttcactgatggcattcacagcctctatcaccagcccgctcaccacccagcataccctcttgtttaacatcttgggggggtgggtggcagcccaactcgccccccccagcgctgcttcagctttcgtgggcgccgggatcgctggcgcggctgttggcagcataggccttgggaaggtgcttgtggacatcctggcgggctatggggcgggggtggcaggcgcgctcgtggcctttaaggtcatgagcggcgaaatgccctccaccgaggacctagtcaacttgctccctgccatcctctcccccggtgccctggttgtcggggtcgtgtgcgcggcaatactgcgccggcacgtgggcccgggggagggggctgtgcagtggatgaaccggctgatagcgttcgcttcgcggggtaaccatgtttcccccacgcactatgtgcctgagagcgacgcggccgcgcgtgtcactcagatcctctccagccttaccatcacccagctgttgaagagactccaccagtggatcaatgaggactgctccacgccatgctccggctcgtggctcagggatgtttgggactggatatgcacggtgttgactgacttcaagacctggctccagtccaagctcctgccacggttgccgggggtccctttcctttcatgccaacgtggatataagggagtctggcgaggagatggcgtcatgcacaccacttgcccatgtggcgcacaaatcaccggacatgtcaaaaacggttccatgaggatcgttgggcctaaaacctgtagcaacacgtggcacggaacattccccatcaacgcgtacaccacgggttcctgcacaccctccccggcgccaaactattccagggcgttgtggcgggtagctgctgaggagtacgtggaggtcacgcgggtgggggatttccactacgtgacgggcatgaccactgacaacataaaatgcccatgccaggttccggcccccgaatttttcacagagttggacggggtgcggctgcacaggtacgctccggcgtgcaaacctctcttacgggatgaggtcacattccaggtcgggctcaaccaatacgtggttgggtcacagcttccatgcgagcccgaaccggatgtaacagtgctcacttccatgctcaccgacccctcccacattacagcagagacggctaagcgtaggctggccagggggtctcccccttccttggccagctcttcagctagtcagttgtctgcgccttctctgaaggcgacatgcaccacccatcatgactccccagacgccgacctcatcgaggccaacctcctgtggcggcaggagatgggcgggaacatcacccgcgtggagtcagagaacaaagtagtgattctggactctttcgacccgcttcgagcggaggaggatgagagggaagtatccgttccggcggagatcctgcggaaaaccaggaaattcccctcagcgttgcccatatgggcacgcccggactacaacccaccactgttagagtcttggagagacccggactatgtccctccggtggtacacgggtgcccgttaccgcccaccaaggcccctccaataccacctccacggagaaagagaacagttatcctgacagaatccactgtgtcttctgccttggcggagcttgctacaaagaccttcggcagctccggatcgtcggccgtcgacagcggcacagcgactgcccctcctgatgggccctccgacgacggtgatgcaggatcggacgctgagtcatactcctccatgcccccccttgagggggagccgggggatcccgatctcagcgacgggtcttggtccactgtgagcgaggaggccagtgaggacgtcgtctgctgctcaatgtcctacacatggacaggtgccttaatcacgccatgcgccgcggaggagagcaagctgcccatcaatgcgttgagcaactctttgctgcgccaccacaacatggtctatgctacaacatcccgcagcgcaagccagcggcagaaaaaggtcacctttgacagactgcaagtcctggacgaccactaccgggacgtgctcaaggagatgaaggcgaaggcgtccacagttaaggccaaacttctatccgtagaagaggcctgcaagctgacgcccccacattcagccagatctaagtttggctatggggcaaaggacgtccggaacctatccagtaaggccgttaaccacatccactccgtgtggaaggacttgctggaagacactgagacaccaattgataccaccgtcatggcaaaaaatgaggtcttctgtgtccaaccagagaaaggaggccgcaagccagctcgccttatcgtgttcccagacctgggggtgcgtgtgtgcgagaaaatggccctttatgatgtggtctccactcttcctcaggccgtgatgggctcctcatacggattccaatactctcctggacagcgggtcgagttcctggtgaatgcctggaaatcaaagaagacccctatgggcttcgcatatgacacccgctgttttgactcaacagtcactgagaatgacatccgtgttgaggagtcaatttaccaatgttgtgacttggcccccgaggccagacaggccataaggtcgctcacagagcggctttatatcgggggccccctgactaattcaaaagggcagaactgcggttatcgccggtgccgcgccagcggcgtgctgacgactagctgcggtaacaccctcacatgttacttgaaggcctctgcagcctgtcgagctgcaaagctccaggactgcacgatgctcgtgtgcggagacgacctcgtcgttatctgtgagagcgcggggacccaggaggacgcggcgagcctgcgagtcttcacggaggctatgaccaggtactctgccccccccggggacccgccccaaccggaatacgacttggagctgataacatcatgctcctccaacgtgtcggtcgcgcacgatgcatctggcaagcgggtgtactatctcacccgcgaccccaccacccctcttgctcgggctgcgtgggagacagctagacacactccagtcaactcctggctaggcaacatcatcatgtatgcgcccaccttatgggcaaggatgattctgatgacccacttcttctcaatccttctagcccaggagcaacttgagaaagccctagattgccagatctacggggccacttactccattgagccacttgacctacctcagatcattcaacgactccatggtcttagcgcattttcactccatagttactctccaggtgagatcaatagggtggcttcatgcctcaggaaacttggggtaccgcccttgcgagtctggagacatcgggccagaagtgtccgcgctaagctactgtcccagggggggagggccgccacgtgtggcaagtacctcttcaactgggccgtacggaccaagctcaaactcactccaattccggctgcgtcccggttggacttgtccggctggttcgtcgccggttacagcgggggagacatatatcacagcctgtctcgcgcccgaccccgctggttcatgtggtgcctactcctactttccgtaggggtaggcatctacctgctccccaaccggtgaacggggagctaaacactccaggccaataggccattcctttttttttttttttttttttttttctttttttttccttttttttttttttttttttttttccttttctttcttctttggtggctccatcttagccctagtcacggctagctgtgaaaggtccgtgagccgcatgactgcagagagtgctgatactggcctctctgcagatcatgt

SEQ ID NO: 2 - protein, 3010 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNSSIVYEAADMIMHTPGCVPCVREDNSSRCWVALTPTLAARNSSVPTTTIRRHVDLLVGAAAFCSAMYVGDLCGSVFLISQLFTFSPRRYETVQDCNCSLYPGHVSGHRMAWDMMMNWSPTTALVVSQLLRIPQAVVDMVAGAHWGVLAGLAYYSMVGNWAKVLIVMLLFAGVDGNTRVSGGEAAKNTMGFASLFVSGPSQKIQLINTNGSWHINRTALNCDDSLHTGFLAALFYAHKFNSSGCSGRMASCRPIDEFAQGWGPITHGVPDNLDQRPYCWHYAPRPCGIVPASQVCGPVYCFTPSPVVVGTTDRFGAPTYSWGENETDVLLLNNTRPPQGNWFGCTWMNSTGFTKTCGGPPCNIGGVGNNTLTCPTDCFRKHPEATYTKCGSGPWLTPRCMVDYPYRLWHYPCTVNFTIFKVRMYVGGVEHRLDAACNWTRGERCNVEDRDRSELSPLLLSTTEWQILPCSFTTLPALSTGLIHLHQNIVDVQYLYGVGSVVVSVVIRWEYVVLLFLLLADARVCACLWMMLLIAQAEAALENLVVLNAASVAGAHGTLSFLVFFCAAWYIKGKLVPGAAYAFYGVWPLLLLLLALPPRAYAMDREMAASCGGAVFIGLALLTLSPHYKAFLARLIWWLQYLITRVEALLQVWVPPLNVRGGRDAIILLTCAVHPELIFEITKILLAIIGPLMVLQAGLTRVPYFVRAQGLIRVCMLVRKVAGGHYVQMALMRLAALTGTYVYNHLTPLRDWAHAGLRDLVVAVEPVVFSDMETKIITWGADTAACGDIILGLPVSARRGREILLGPADSYEGQGWRLLAPITAYSQQTRGLLGCIITSLTGRDRNQVEGEVQVVSTATQSFLATCINGVCWTVYHGAGSKTLAGPKGPITQMYTNVDQDLVGWQAPPGARSLTPCTCGSSDLYLVTRHADVIPVRRRGDNRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTSILGIGTVLDQAETAGARLVVLATATPPGSVTVPHPNIEEVALSNTGEIPFYGKAFPIEAIKGGRHLIFCHSKKKCDELATKLSALGINAVAYYRGLDVSVIPTSGDVVVVATDALMTGYTGDFDSVIDCNTCVTQTVDFSLDPTFTIETTTVPQDAVSRSQRRGRTGRGRGGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKFIMACMSADLEVVTSTWVLVGGVLAALAAYCLTTGSVVIVGRIILSGKPAVIPDREVLYQEFDEMEECASHLPYIEQGMQLAEQFKQKALGLLQAATKQAEAAAPVVESKWQALEAFWAKHMWNFISGIQYLAGLSTLPGNPPIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPRLPGVPFLSCQRGYKGVWRGDGVMHTTCPCGAQITGHVKNGSMRIVGPKTCSNTWHGTFPINAYTTGSCTPSPAPNYSRALWRVAAEEYVEVTRVGDFHYVTGMTTDNIKCPCQVPAPEFFTELDGVRLHRYAPACKPLLRDEVTFQVGLNQYVVGSQLPCEPEPDVTVLTSMLTDPSHITAETAKRRLARGSPPSLASSSASQLSAPSLKATCTTHHDSPDADLIEANLLWRQEMGGNITRVESENKVVILDSFDPLRAEEDEREVSVPAEILRKTRKFPSALPIWARPDYNPPLLESWRDPDYVPPVVHGCPLPPTKAPPIPPPRRKRTVILTESTVSSALAELATKTFGSSGSSAVDSGTATAPPDGPSDDGDAGSDAESYSSMPPLEGEPGDPDLSDGSWSTVSEEASEDVVCCSMSYTWTGALITPCAAEESKLPINALSNSLLRHHNMVYATTSRSASQRQKKVTFDRLQVLDDHYRDVLKEMKAKASTVKAKLLSVEEACKLTPPHSARSKFGYGAKDVRNLSSKAVNHIHSVWKDLLEDTETPIDTTVMAKNEVFCVQPEKGGRKPARLIVFPDLGVRVCEKMALYDVVSTLPQAVMGSSYGFQYSPGQRVEFLVNAWKSKKTPMGFAYDTRCFDSTVTENDIRVEESIYQCCDLAPEARQAIRSLTERLYIGGPLTNSKGQNCGYRRCRASGVLTTSCGNTLTCYLKASAACRAAKLQDCTMLVCGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGATYSIEPLDLPQIIQRLHGLSAFSLHSYSPGEINRVASCLRKLGVPPLRVWRHRARSVRAKLLSQGGRAATCGKYLFNWAVRTKLKLTPIPAASRLDLSGWFVAGYSGGDIYHSLSRARPRWFMWCLLLLSVGVGIYLLPNR

SEQ ID NO: 3 - DNA, 6168 bp, Hepatitis C virus
gcgcccatcacggcttactcccaacagacgcggggcctactcggttgcatcatcactagtctcacaggccgggacaggaaccaggtcgagggggaggttcaggtggtttccaccgcaacgcagtccttcttggcgacctgcatcaatggtgtgtgttggactgtctaccatggtgccggctcaaagaccctagccggcccaaagggtccgatcacccaaatgtacaccaatgtagaccaggacctcgtcggctggcgggcgccccccggggcgcgttccttaacaccatgcacctgcggcagctcggacctctacttggtcacgaggcatgctgatgtcattccggtgcgccggcggggcgataacaggggcagcctgctttcccccagacctgtctcttacttaaagggctcctcgggtggtccactgctctgcccctcagggcacgctgtgggcatcttccgggccgccgtgtgcaccaggggggttgcgaaggcggtggactttgtgcccgttgagtccatggaaactaccatgcggtctccggtcttcacggacaattcatctcccccggccgtaccgcagacattccaagtggcccatctacacgctcccactggcagcggtaagagcactaaggtgccggctgcttatgcagcccaagggtacaaggtactcgtcctgaacccatccgttgccgccaccttaggttttggggcgtatatgtctaaagcacatggtgttgaccccaacatcagaactggggtaaggaccatcaccacaggcgctcccattacgtattccacttatggcaagttccttgccgacggtggttgctctgggggcgcctatgacatcataatatgtgatgagtgccactcaactgactcgacttccatcttgggcatcggcactgtcttggaccaagcggagacggctggagcgcggctcgtcgtgctcgccaccgctacacctccgggatcagtcaccgtgccacatcccaatatcgaggaggtggctttgtccaacactggagagatccccttttatggtaaggcctttcccatcgaggccatcaagggggggaggcatctcattttctgccactccaagaagaaatgtgacgagctcgccacaaagctgtcggcccttggaatcaacgctgtagcgtactaccggggtcttgatgtgtccgtcataccgacaagcggagatgtcgttgtcgtggcaacagacgctctgatgacgggctacactggcgactttgactcagtgatcgactgtaacacatgtgtcacccagacagtcgacttcagcttggaccctaccttcaccattgagacgacgaccgtgccccaagacgcggtatcgcgctcgcaacggcgaggcaggactggtaggggcagggggggcatatacaggtttgtgactccaggggaacggccctcgggcatgttcgattcttcggtcctgtgcgagtgctatgacgcgggctgtgcttggtacgagctcacgcccgccgagacctcggttaggttgcgggcttacctaaatacaccagggttgcccgtctgccaggaccatctggagttctgggagagcgtcttcacaggcctcacccacatagatgcccatttcttatcccagaccaagcaggcaggagacaacttcccctacctggtagcgtaccaggctacggtgtgcgccagggcccaggccccaccaccatcgtgggaccaaatgtgggagtgtctcatacggctaaagcctacactgcacgggccaacacccctgctgtacaggctaggagccgtccaaaacgaagtcactctcacacaccccataaccaaattcatcatggcatgcatgtcggctgacctagaggtcgtcacgagcacctgggtgctagtaggcggggtccttgcagctctggccgcgtactgcctgacaacgggcagcgtggtcattgtgggtaggatcatcttgtctgggaagccggctgtcattcccgatagggaagtgctctaccaggagttcgatgaaatggaagagtgcgcctcgcacctcccttacattgaacagggaatgcagctcgccgagcaattcaagcagaaggcgctcgggttgctgcaagcagccaccaagcaagcggaggctgctgctcccgtggtggagtccaagtggcaagcccttgaggccttttgggcgaagcacatgtggaatttcatcagcgggatacagtacttagcaggcttgtccactctgcctgggaaccccccgatagcttcactgatggcattcacagcctctatcaccagcccgctcaccacccagcataccctcttgtttaacatcttgggggggtgggtggcagcccaactcgccccccccagcgctgcttcagctttcgtgggcgccgggatcgctggcgcggctgttggcagcataggccttgggaaggtgcttgtggacatcctggcgggctatggggcgggggtggcaggcgcgctcgtggcctttaaggtcatgagcggcgaaatgccctccaccgaggacctagtcaacttgctccctgccatcctctcccccggtgccctggttgtcggggtcgtgtgcgcggcaatactgcgccggcacgtgggcccgggggagggggctgtgcagtggatgaaccggctgatagcgttcgcttcgcggggtaaccatgtttcccccacgcactatgtgcctgagagcgacgcggccgcgcgtgtcactcagatcctctccagccttaccatcacccagctgttgaagagactccaccagtggatcaatgaggactgctccacgccatgctccggctcgtggctcagggatgtttgggactggatatgcacggtgttgactgacttcaagacctggctccagtccaagctcctgccacggttgccgggggtccctttcctttcatgccaacgtggatataagggagtctggcgaggagatggcgtcatgcacaccacttgcccatgtggcgcacaaatcaccggacatgtcaaaaacggttccatgaggatcgttgggcctaaaacctgtagcaacacgtggcacggaacattccccatcaacgcgtacaccacgggttcctgcacaccctccccggcgccaaactattccagggcgttgtggcgggtagctgctgaggagtacgtggaggtcacgcgggtgggggatttccactacgtgacgggcatgaccactgacaacataaaatgcccatgccaggttccggcccccgaatttttcacagagttggacggggtgcggctgcacaggtacgctccggcgtgcaaacctctcttacgggatgaggtcacattccaggtcgggctcaaccaatacgtggttgggtcacagcttccatgcgagcccgaaccggatgtaacagtgctcacttccatgctcaccgacccctcccacattacagcagagacggctaagcgtaggctggccagggggtctcccccttccttggcccgctcttcagctagtcagttgtctgcgccttctctgaaggcgacatgcaccacccatcatgactccccagacgccgacctcatcgaggccaacctcctgtggcggcaggagatgggcgggaacatcacccgcgtggagtcagagaacaaagtagtgattctggactctttcgacccgcttcgagcggaggaggatgagagggaagtatccgttccggcggagatcctgcggaaaaccaggaaattcccctcagcgttgcccatatgggcacgcccggactacaacccaccactgttagagtcttggagagacccggactatgtccctccggtggtacacgggtgcccgttaccgcccaccaaggcccctccaataccacctccacggagaaagagaacagttatcctgacagaatccactgtgtcttctgccttggcggagcttgctacaaagaccttcggcagctccggatcgtcggccgtcgacagcggcacagcgactgcccctcctgatgggccctccgacgacggtgatgcaggatcggacgctgagtcatactcctccatgcccccccttgagggggagccgggggatcccgatctcagcgacgggtcttggtccactgtgagcgaggaggccagtgaggacgtcgtctgctgctcaatgtcctacacatggacaggtgccttaatcacgccatgcgccgcggaggagagcaagctgcccatcaatgcgttgagcaactctttgctgcgccaccacaacatggtctatgctacaacatcccgcagcgcaagccagcggcagaaaaaggtcacctttgacagactgcaagtcctggacgaccactaccgggacgtgctcaaggagatgaaggcgaaggcgtccacagttaaggccaaacttctatccgtagaagaggcctgcaagctgacgcccccacattcagccagatctaagtttggctatggggcaaaggacgtccggaacctatccagtaaggccgttaaccacatccactccgtgtggaaggacttgctggaagacactgagacaccaattgataccaccgtcatggcaaaaaatgaggtcttctgtgtccaaccagagaaaggaggccgcaagccagctcgccttatcgtgttcccagacctgggggtgcgtgtgtgcgagaaaatggccctttatgatgtggtctccactcttcctcaggccgtgatgggctcctcatacggattccaatactctcctggacagcgggtcgagttcctggtgaatgcctggaaatcaaagaagacccctatgggcttcgcatatgacacccgctgttttgactcaacagtcactgagaatgacatccgtgttgaggagtcaatttaccaatgttgtgacttggcccccgaggccagacaggccataaggtcgctcacagagcggctttatatcgggggccccctgactaattcaaaagggcagaactgcggttatcgccggtgccgcgccagcggcgtgctgacgactagctgcggtaacaccctcacatgttacttgaaggcctctgcagcctgtcgagctgcaaagctccaggactgcacgatgctcgtgtgcggagacgacctcgtcgttatctgtgagagcgcggggacccaggaggacgcggcgagcctgcgagtcttcacggaggctatgaccaggtactctgccccccccggggacccgccccaaccggaatacgacttggagctgataacatcatgctcctccaacgtgtcggtcgcgcacgatgcatctggcaagcgggtgtactatctcacccgcgaccccaccacccctcttgctcgggctgcgtgggagacagctagacacactccagtcaactcctggctaggcaacatcatcatgtatgcgcccaccttatgggcaaggatgattctgatgacccacttcttctcaatccttctagcccaggagcaacttgagaaagccctagattgccagatctacggggccacttactccattgagccacttgacctacctcagatcattcaacgactccatggtcttagcgcattttcactccatagttactctccaggtgagatcaatagggtggcttcatgcctcaggaaacttggggtaccgcccttgcgagtctggagacatcgggccagaagtgtccgcgctaagctactgtcccagggggggagggccgccacgtgtggcaagtacctcttcaactgggccgtacggaccaagctcaaactcactccaattccggctgcgtcccggttggacttgtccggctggttcgtcgccggttacagcgggggagacatatatcacagcctgtctcgcgcccgaccccgctggttcatgtggtgcctactcctactttccgtaggggtaggcatctacctgctccccaaccggtgaacggggagctaaacactccaggccaataggccattcctttttttttttttttttttttttttctttttttttccttttttttttttttttttttttttccttttctttcttctttggtggctccatcttagccctagtcacggctagctgtgaaaggtccgtgagccgcatgactgcagagagtgctgatactggcctctctgcagatcatgt

SEQ ID NO: 4 - protein, 1984 aa, Hepatitis C virus
APITAYSQQTRGLLGCIITSLTGRDRNQVEGEVQVVSTATQSFLATCINGVCWTVYHGAGSKTLAGPKGPITQMYTNVDQDLVGWRAPPGARSLTPCTCGSSDLYLVTRHADVIPVRRRGDNRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTSILGIGTVLDQAETAGARLVVLATATPPGSVTVPHPNIEEVALSNTGEIPFYGKAFPIEAIKGGRHLIFCHSKKKCDELATKLSALGINAVAYYRGLDVSVIPTSGDVVVVATDALMTGYTGDFDSVIDCNTCVTQTVDFSLDPTFTIETTTVPQDAVSRSQRRGRTGRGRGGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWECLIRLKPTLHGPTPLLYRLGAVQNEVTLTHPITKFIMACMSADLEVVTSTWVLVGGVLAALAAYCLTTGSVVIVGRIILSGKPAVIPDREVLYQEFDEMEECASHLPYIEQGMQLAEQFKQKALGLLQAATKQAEAAAPVVESKWQALEAFWAKHMWNFISGIQYLAGLSTLPGNPPIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPRLPGVPFLSCQRGYKGVWRGDGVMHTTCPCGAQITGHVKNGSMRIVGPKTCSNTWHGTFPINAYTTGSCTPSPAPNYSRALWRVAAEEYVEVTRVGDFHYVTGMTTDNIKCPCQVPAPEFFTELDGVRLHRYAPACKPLLRDEVTFQVGLNQYVVGSQLPCEPEPDVTVLTSMLTDPSHITAETAKRRLARGSPPSLARSSASQLSAPSLKATCTTHHDSPDADLIEANLLWRQEMGGNITRVESENKVVILDSFDPLRAEEDEREVSVPAEILRKTRKFPSALPIWARPDYNPPLLESWRDPDYVPPVVHGCPLPPTKAPPIPPPRRKRTVILTESTVSSALAELATKTFGSSGSSAVDSGTATAPPDGPSDDGDAGSDAESYSSMPPLEGEPGDPDLSDGSWSTVSEEASEDVVCCSMSYTWTGALITPCAAEESKLPINALSNSLLRHHNMVYATTSRSASQRQKKVTFDRLQVLDDHYRDVLKEMKAKASTVKAKLLSVEEACKLTPPHSARSKFGYGAKDVRNLSSKAVNHIHSVWKDLLEDTETPIDTTVMAKNEVFCVQPEKGGRKPARLIVFPDLGVRVCEKMALYDVVSTLPQAVMGSSYGFQYSPGQRVEFLVNAWKSKKTPMGFAYDTRCFDSTVTENDIRVEESIYQCCDLAPEARQAIRSLTERLYIGGPLTNSKGQNCGYRRCRASGVLTTSCGNTLTCYLKASAACRAAKLQDCTMLVCGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGATYSIEPLDLPQIIQRLHGLSAFSLHSYSPGEINRVASCLRKLGVPPLRVWRHRARSVRAKLLSQGGRAATCGKYLFNWAVRTKLKLTPIPAASRLDLSGWFVAGYSGGDIYHSLSRARPRWFMWCLLLLSVGVGIYLLPNR

SEQ ID NO: 5 - DNA, 6168 bp, Hepatitis C virus
gcgcccatcacggcttactcccaacagacgcggggcctactcggttgcatcatcactagtctcacaggccgggacaggaaccaggtcgagggggaggttcaggtggtttccaccgcaacgcagtccttcttggcgacctgcatcaatggtgtgtgttggactgtctaccatggtgccggctcaaagaccctagccggcccaaagggtccgatcacccaaatgtacaccaatgtagaccaggacctcgtcggctggcgggcgcccctcggggcgcgttccttaacaccatgcacctgcggcagctcggacctctacttggtcacgaggcatgctgatgtcattccggtgcgccggcggggcgataacaggggcagcctgctttcccccagacctgtctcttacttaaagggctcctcgggtggtccactgctctgcccctcagggcacgctgtgggcatcttccgggccgccgtgtgcaccaggggggttgcgaaggcggtggactttgtgcccgttgagtccatggaaactaccatgcggtctccggtcttcacggacaattcatctcccccggccgtaccgcagacattccaagtggcccatctacacgctcccactggcagcggtaagagcactaaggtgccggctgcttatgcagcccaagggtacaaggtactcgtcctgaacccatccgttgccgccaccttaggttttggggcgtatatgtctaaagcacatggtgttgaccccaacatcagaactggggtaaggaccatcaccacaggcgctcccattacgtattccacttatggcaagttccttgccgacggtggttgctctgggggcgcctatgacatcataatatgtgatgagtgccactcaactgactcgacttccatcttgggcatcggcactgtcttggaccaagcggagacggctggagcgcggctcgtcgtgctcgccaccgctacacctccgggatcagtcaccgtgccacatcccaatatcgaggaggtggctttgtccaacactggagagatccccttttatggtaaggcctttcccatcgaggccatcaagggggggaggcatctcattttctgccactccaagaagaaatgtgacgagctcgccacaaagctgtcggcccttggaatcaacgctgtagcgtactaccggggtcttgatgtgtccgtcataccgacaagcggagatgtcgttgtcgtggcaacagacgctctgatgacgggctacactggcgactttgactcagtgatcgactgtaacacatgtgtcacccagacagtcgacttcagcttggaccctaccttcaccattgagacgacgaccgtgccccaagacgcggtatcgcgctcgcaacggcgaggcaggactggtaggggcagggggggcatatacaggtttgtgactccaggggaacggccctcgggcatgttcgattcttcggtcctgtgcgagtgctatgacgcgggctgtgcttggtacgagctcacgcccgccgagacctcggttaggttgcgggcttacctaaatacaccagggttgcccgtctgccaggaccatctggagttctgggagagcgtcttcacaggcctcacccacatagatgcccatttcttatcccagaccaagcaggcaggagacaacttcccctacctggtagcgtaccaggctacggtgtgcgccagggcccaggccccaccaccatcgtgggaccaaatgtggaagtgtctcatacggctaaagcctacactgcacgggccaacacccctgctgtacaggctaggagccgtccaaaacgaagtcactctcacacaccccataaccaaattcatcatggcatgcatgtcggctgacctagaggtcgtcacgagcacctgggtgctagtaggcggggtccttgcagctctggccgcgtactgcctgacaacgggcagcgtggtcattgtgggtaggatcatcttgtctgggaagccggctgtcattcccgatagggaagtgctctaccaggagttcgatgaaatggaagagtgcgcctcgcacctcccttacattgaacagggaatgcagctcgccgagcaattcaagcagaaggcgctcgggttgctgcaagcagccaccaagcaagcggaggctgctgctcccgtggtggagtccaagtggcaagcccttgaggccttttgggcgaagcacatgtggaatttcatcagcgggatacagtacttagcaggcttgtccactctgcctgggaaccccccgatagcttcactgatggcattcacagcctctatcaccagcccgctcaccacccagcataccctcttgtttaacatcttgggggggtgggtggcagcccaactcgccccccccagcgctgcttcagctttcgtgggcgccgggatcgctggcgcggctgttggcagcataggccttgggaaggtgcttgtggacatcctggcgggctatggggcgggggtggcaggcgcgctcgtggcctttaaggtcatgagcggcgaaatgccctccaccgaggacctagtcaacttgctccctgccatcctctcccccggtgccctggttgtcggggtcgtgtgcgcggcaatactgcgccggcacgtgggcccgggggagggggctgtgcagtggatgaaccggctgatagcgttcgcttcgcggggtaaccatgtttcccccacgcactatgtgcctgagagcgacgcggccgcgcgtgtcactcagatcctctccagccttaccatcacccagctgttgaagagactccaccagtggatcaatgaggactgctccacgccatgctccggctcgtggctcagggatgtttgggactggatatgcacggtgttgactgacttcaagacctggctccagtccaagctcctgccacggttgccgggggtccctttcctttcatgccaacgtggatataagggagtctggcgaggagatggcgtcatgcacaccacttgcccatgtggcgcacaaatcaccggacatgtcaaaaacggttccatgaggatcgttgggcctaaaacctgtagcaacacgtggcacggaacattccccatcaacgcgtacaccacgggttcctgcacaccctccccggcgccaaactattccagggcgttgtggcgggtagctgctgaggagtacgtggaggtcacgcgggtgggggatttccactacgtgacgggcatgaccactgacaacataaaatgcccatgccaggttccggcccccgaatttttcacagagttggacggggtgcggctgcacaggtacgctccggcgtgcaaacctctcttacgggatgaggtcacattccaggtcgggctcaaccaatacgtggttgggtcacagcttccatgcgagcccgaaccggatgtaacagtgctcacttccatgctcaccgacccctcccacattacagcagagacggctaagcgtaggctggccagggggtctcccccttccttggcccgctcttcagctagtcagttgtctgcgccttctctgaaggcgacatgcaccacccatcatgactccccagacgccgacctcatcgaggccaacctcctgtggcggcaggagatgggcgggaacatcacccgcgtggagtcagagaacaaagtagtgattctggactctttcgacccgcttcgagcggaggaggatgagagggaagtatccgttccggcggagatcctgcggaaaaccaggaaattcccctcagcgttgcccatatgggcacgcccggactacaacccaccactgttagagtcttggagagacccggactatgtccctccggtggtacacgggtgcccgttaccgcccaccaaggcccctccaataccacctccacggagaaagagaacagttatcctgacagaatccactgtgtcttctgccttggcggagcttgctacaaagaccttcggcagctccggatcgtcggccgtcgacagcggcacagcgactgcccctcctgatgggccctccgacgacggtgatgcaggatcggacgctgagtcatactcctccatgcccccccttgagggggagccgggggatcccgatctcagcgacgggtcttggtccactgtgagcgaggaggccagtgaggacgtcgtctgctgctcaatgtcctacacatggacaggtgccttaatcacgccatgcgccgcggaggagagcaagctgcccatcaatgcgttgagcaactctttgctgcgccaccacaacatggtctatgctacaacatcccgcagcgcaagccagcggcagaaaaaggtcacctttgacagactgcaagtcctggacgaccactaccgggacgtgctcaaggagatgaaggcgaaggcgtccacagttaaggccaaacttctatccgtagaagaggcctgcaagctgacgcccccacattcagccagatctaagtttggctatggggcaaaggacgtccggaacctatccagtaaggccgttaaccacatccactccgtgtggaaggacttgctggaagacactgagacaccaattgataccaccgtcatggcaaaaaatgaggtcttctgtgtccaaccagagaaaggaggccgcaagccagctcgccttatcgtgttcccagacctgggggtgcgtgtgtgcgagaaaatggccctttatgatgtggtctccactcttcctcaggccgtgatgggctcctcatacggattccaatactctcctggacagcgggtcgagttcctggtgaatgcctggaaatcaaagaagacccctatgggcttcgcatatgacacccgctgttttgactcaacagtcactgagaatgacatccgtgttgaggagtcaatttaccaatgttgtgacttggcccccgaggccagacaggccataaggtcgctcacagagcggctttatatcgggggccccctgactaattcaaaagggcagaactgcggttatcgccggtgccgcgccagcggcgtgctgacgactagctgcggtaacaccctcacatgttacttgaaggcctctgcagcctgtcgagctgcaaagctccaggactgcacgatgctcgtgtgcggagacgacctcgtcgttatctgtgagagcgcggggacccaggaggacgcggcgagcctgcgagtcttcacggaggctatgaccaggtactctgccccccccggggacccgccccaaccggaatacgacttggagctgataacatcatgctcctccaacgtgtcggtcgcgcacgatgcatctggcaagcgggtgtactatctcacccgcgaccccaccacccctcttgctcgggctgcgtgggagacagctagacacactccagtcaactcctggctaggcaacatcatcatgtatgcgcccaccttatgggcaaggatgattctgatgacccacttcttctcaatccttctagcccaggagcaacttgagaaagccctagattgccagatctacggggccacttactccattgagccacttgacctacctcagatcattcaacgactccatggtcttagcgcattttcactccatagttactctccaggtgagatcaatagggtggcttcatgcctcaggaaacttggggtaccgcccttgcgagtctggagacatcgggccagaagtgtccgcgctaagctactgtcccagggggggagggccgccacgtgtggcaagtacctcttcaactgggccgtacggaccaagctcaaactcactccaattccggctgcgtcccggttggacttgtccggctggttcgtcgccggttacagcgggggagacatatatcacagcctgtctcgcgcccgaccccgctggttcatgtggtgcctactcctactttccgtaggggtaggcatctacctgctccccaaccggtgaacggggagctaaacactccaggccaataggccattcctttttttttttttttttttttttttctttttttttccttttttttttttttttttttttttccttttctttcttctttggtggctccatcttagccctagtcacggctagctgtgaaaggtccgtgagccgcatgactgcagagagtgctgatactggcctctctgcagatcatgt

SEQ ID NO: 6 - protein, 1984 aa, Hepatitis C virus
APITAYSQQTRGLLGCIITSLTGRDRNQVEGEVQVVSTATQSFLATCINGVCWTVYHGAGSKTLAGPKGPITQMYTNVDQDLVGWRAPLGARSLTPCTCGSSDLYLVTRHADVIPVRRRGDNRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTSILGIGTVLDQAETAGARLVVLATATPPGSVTVPHPNIEEVALSNTGEIPFYGKAFPIEAIKGGRHLIFCHSKKKCDELATKLSALGINAVAYYRGLDVSVIPTSGDVVVVATDALMTGYTGDFDSVIDCNTCVTQTVDFSLDPTFTIETTTVPQDAVSRSQRRGRTGRGRGGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKFIMACMSADLEVVTSTWVLVGGVLAALAAYCLTTGSVVIVGRIILSGKPAVIPDREVLYQEFDEMEECASHLPYIEQGMQLAEQFKQKALGLLQAATKQAEAAAPVVESKWQALEAFWAKHMWNFISGIQYLAGLSTLPGNPPIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPRLPGVPFLSCQRGYKGVWRGDGVMHTTCPCGAQITGHVKNGSMRIVGPKTCSNTWHGTFPINAYTTGSCTPSPAPNYSRALWRVAAEEYVEVTRVGDFHYVTGMTTDNIKCPCQVPAPEFFTELDGVRLHRYAPACKPLLRDEVTFQVGLNQYVVGSQLPCEPEPDVTVLTSMLTDPSHITAETAKRRLARGSPPSLARSSASQLSAPSLKATCTTHHDSPDADLIEANLLWRQEMGGNITRVESENKVVILDSFDPLRAEEDEREVSVPAEILRKTRKFPSALPIWARPDYNPPLLESWRDPDYVPPVVHGCPLPPTKAPPIPPPRRKRTVILTESTVSSALAELATKTFGSSGSSAVDSGTATAPPDGPSDDGDAGSDAESYSSMPPLEGEPGDPDLSDGSWSTVSEEASEDVVCCSMSYTWTGALITPCAAEESKLPINALSNSLLRHHNMVYATTSRSASQRQKKVTFDRLQVLDDHYRDVLKEMKAKASTVKAKLLSVEEACKLTPPHSARSKFGYGAKDVRNLSSKAVNHIHSVWKDLLEDTETPIDTTVMAKNEVFCVQPEKGGRKPARLIVFPDLGVRVCEKMALYDVVSTLPQAVMGSSYGFQYSPGQRVEFLVNAWKSKKTPMGFAYDTRCFDSTVTENDIRVEESIYQCCDLAPEARQAIRSLTERLYIGGPLTNSKGQNCGYRRCRASGVLTTSCGNTLTCYLKASAACRAAKLQDCTMLVCGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGATYSIEPLDLPQIIQRLHGLSAFSLHSYSPGEINRVASCLRKLGVPPLRVWRHRARSVRAKLLSQGGRAATCGKYLFNWAVRTKLKLTPIPAASRLDLSGWFVAGYSGGDIYHSLSRARPRWFMWCLLLLSVGVGIYLLPNR

SEQ ID NO: 7 - DNA, 9587 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggattaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgactaggaagacttccgagcggtcgcagcctcgtggaaggcgacaacctatccccaaggctcgccagcccgagggcagggcctgggctcagcccggatacccttggcccctctacggcaatgagggtctggggtgggcaggatggctcctgtcaccccgtggctctcggcctagttggggccctacggacccccggcgtaggtcgcgtaatttgggtaaggtcatcgataccctcacatgcggcttcgccgacctcatggggtacatcccgctcgtcggcgcccccctaggaggcgctgccagggccctggcgcatggcgtccgggttctggaggacggcgtgaactatgcaacagggaatttgcccggttgctctttctctatcttcctcttggctttgctgtcctgtttgaccatcccagcttccgcttacgaagtgcgcaacgtatccggggtgtaccatgtcacgaacgactgctccaactcaagtattgtgtatgaggcagcggacatgatcatgcacacccccgggtgcgtgccctgcgttcgggaggacaattcctcccgttgctgggtagcgctcactcccacgcttgcggccaggaacagcagcgtccccaccacgacaatacgacgccacgtcgacttgctcgttggggcagctgctttctgttccgctatgtacgtgggagacctctgcggatctgttttcctcatttcccagctgttcaccttctcacctcgccggtatgagacggtgcaagactgcaactgctcgctctatcccggccacgtatcaggtcaccgcatggcttgggatatgatgatgaactggtcacctacaacagccctggtggtgtcgcagttgctccggatcccacaagccgtcgtggacatggtggcgggggcccactggggagtcctggcgggccttgcctactattccatggtggggaactgggctaaggtcttgattgtgatgctactttttgccggcgtcgacgggaacactcgtgtgtcagggggggaagcagccaagaacaccatgggctttgcatccctctttgtgtctggcccgtcgcagaaaatccaacttattaacaccaatggcagctggcacatcaacaggactgctctgaattgcgatgactccctccacaccgggttccttgctgcgctgttttacgcgcacaagttcaactcgtccggatgctcggggcgcatggccagctgccgccccattgacgagtttgcccaggggtggggtcccatcactcacggtgtgcctgacaacctggaccagaggccttactgctggcactacgcgcctcggccgtgcggtatcgtacccgcgtcgcaggtgtgtggcccagtgtattgcttcaccccgagccctgtcgtggtggggacgaccgatcgtttcggcgcccccacgtacagctggggggagaatgagacggacgtgctactccttaacaacacgcgaccgccacaaggcaactggttcggttgcacatggatgaacagcaccgggttcaccaagacgtgcgggggccccccgtgcaacattggaggggtcggcaacaacaccttgacctgtcctacggactgcttccggaagcaccccgaggccacttacaccaaatgcggctcgggcccttggttgacacctaggtgcatggttgactacccatacagactctggcactacccttgcactgtcaattttaccatcttcaaggtcaggatgtatgtagggggtgtggagcacaggctcgacgccgcgtgcaattggacccgaggagagcgttgcaatgtggaggacagggatagatcagagcttagcccactgctactgtccacaacagagtggcagatactgccctgttccttcaccaccctaccggctctgtccactggtttgatccacctccaccagaacatcgtggacgtgcaatacctgtacggtgtggggtcagtggttgtctccgttgtaatcagatgggagtatgtcgtgctgctcttccttctcctggcggacgcacgcgtctgcgcctgcttgtggatgatgctgctgatagctcaggctgaggccgccttagagaacttggtggtcctcaatgcggcatctgtagctggagcgcatggcactctctccttccttgtgttcttctgtgccgcctggtacatcaagggcaagctggtccctggagcggcatatgctttctatggcgtgtggccgctgctcctgctcctgctggcattaccaccacgagcatacgccatggaccgggagatggctgcatcatgcggaggcgcggtcttcataggtctggcactcctgaccttgtcaccacactataaagcgttcctcgctaggctcatttggtggttgcagtatcttatcaccagggtcgaggcgctactgcaagtgtgggtcccccctcttaacgttcgggggggccgcgatgccatcatcctcctcacgtgcgcggtccatccagagctaatctttgaaatcaccaaaatcttgctcgccataattggtccgctcatggtgctccaggctggcttaaccagagtgccgtacttcgtgcgcgctcaggggctcatccgtgtgtgcatgttggtgcggaaagtcgctgggggtcactacgtccagatggctctcatgaggctggccgcgctgacgggcacgtacgtttacaaccatcttactccgctgcgggactgggcccacgcgggcttgcgggaccttgtggtggcagttgagcccgtcgtcttctctgacatggagaccaagatcatcacctggggggcagacaccgcggcgtgtggggacatcatcttgggtctacccgtctccgcccgaagggggagggagatacttttgggaccggccgacagttatgaagggcagggatggcggcttcttgcgcctatcacggcctattcccaacagacgcggggcctactcggttgcatcatcactagtctcacaggccgggacaggaaccaggtcgagggggaggttcaggtggtttccaccgcaacgcagtccttcttggcgacctgcatcaatggtgtgtgttggactgtctaccatggtgccggctcaaagaccctagccggcccaaagggtccgatcacccaaatgtacaccaatgtagaccaggacctcgtcggctggcgggcgccccccggggcgcgttccttaacaccatgcacctgcggcagctcggacctctacttggtcacgaggcatgctgatgtcattccggtgcgccggcggggcgataacaggggcagcctgctttcccccagacctgtctcttacttaaagggctcctcgggtggtccactgctctgcccctcagggcacgctgtgggcatcttccgggccgccgtgtgcaccaggggggttgcgaaggcggtggactttgtgcccgttgagtccatggaaactaccatgcggtctccggtcttcacggacaattcatctcccccggccgtaccgcagacattccaagtggcccatctacacgctcccactggcagcggtaagagcactaaggtgccggctgcttatgcagcccaagggtacaaggtactcgtcctgaacccatccgttgccgccaccttaggttttggggcgtatatgtctaaagcacatggtgttgaccccaacatcagaactggggtaaggaccatcaccacaggcgctcccattacgtattccacttatggcaagttccttgccgacggtggttgctctgggggcgcctatgacatcataatatgtgatgagtgccactcaactgactcgacttccatcttgggcatcggcactgtcttggaccaagcggagacggctggagcgcggctcgtcgtgctcgccaccgctacacctccgggatcagtcaccgtgccacatcccaatatcgaggaggtggctttgtccaacactggagagatccccttttatggtaaggcctttcccatcgaggccatcaagggggggaggcatctcattttctgccactccaagaagaaatgtgacgagctcgccacaaagctgtcggcccttggaatcaacgctgtagcgtactaccggggtcttgatgtgtccgtcataccgacaagcggagatgtcgttgtcgtggcaacagacgctctgatgacgggctacactggcgactttgactcagtgatcgactgtaacacatgtgtcacccagacagtcgacttcagcttggaccctaccttcaccattgagacgacgaccgtgccccaagacgcggtatcgcgctcgcaacggcgaggcaggactggtaggggcagggggggcatatacaggtttgtgactccaggggaacggccctcgggcatgttcgattcttcggtcctgtgcgagtgctatgacgcgggctgtgcttggtacgagctcacgcccgccgagacctcggttaggttgcgggcttacctaaatacaccagggttgcccgtctgccaggaccatctggagttctgggagagcgtcttcacaggcctcacccacatagatgcccatttcttatcccagaccaagcaggcaggagacaacttcccctacctggtagcgtaccaggctacggtgtgcgccagggcccaggccccaccaccatcgtgggaccaaatgtgggagtgtctcatacggctaaagcctacactgcacgggccaacacccctgctgtacaggctaggagccgtccaaaacgaagtcactctcacacaccccataaccaaattcatcatggcatgcatgtcggctgacctagaggtcgtcacgagcacctgggtgctagtaggcggggtccttgcagctctggccgcgtactgcctgacaacgggcagcgtggtcattgtgggtaggatcatcttgtctgggaagccggctgtcattcccgatagggaagtgctctaccaggagttcgatgaaatggaagagtgcgcctcgcacctcccttacattgaacagggaatgcagctcgccgagcaattcaagcagaaggcgctcgggttgctgcaagcagccaccaagcaagcggaggctgctgctcccgtggtggagtccaagtggcaagcccttgaggccttttgggcgaagcacatgtggaatttcatcagcgggatacagtacttagcaggcttgtccactctgcctgggaaccccccgatagcttcactgatggcattcacagcctctatcaccagcccgctcaccacccagcataccctcttgtttaacatcttgggggggtgggtggcagcccaactcgccccccccagcgctgcttcagctttcgtgggcgccgggatcgctggcgcggctgttggcagcataggccttgggaaggtgcttgtggacatcctggcgggctatggggcgggggtggcaggcgcgctcgtggcctttaaggtcatgagcggcgaaatgccctccaccgaggacctagtcaacttgctccctgccatcctctcccccggtgccctggttgtcggggtcgtgtgcgcggcaatactgcgccggcacgtgggcccgggggagggggctgtgcagtggatgaaccggctgatagcgttcgcttcgcggggtaaccatgtttcccccacgcactatgtgcctgagagcgacgcggccgcgcgtgtcactcagatcctctccagccttaccatcacccagctgttgaagagactccaccagtggatcaatgaggactgctccacgccatgctccggctcgtggctcagggatgtttgggactggatatgcacggtgttgactgacttcaagacctggctccagtccaagctcctgccacggttgccgggggtccctttcctttcatgccaacgtggatataagggagtctggcgaggagatggcgtcatgcacaccacttgcccatgtggcgcacaaatcaccggacatgtcaaaaacggttccatgaggatcgttgggcctaaaacctgtagcaacacgtggcacggaacattccccatcaacgcgtacaccacgggttcctgcacaccctccccggcgccaaactattccagggcgttgtggcgggtagctgctgaggagtacgtggaggtcacgcgggtgggggatttccactacgtgacgggcatgaccactgacaacataaaatgcccatgccaggttccggcccccgaatttttcacagagttggacggggtgcggctgcacaggtacgctccggcgtgcaaacctctcttacgggatgaggtcacattccaggtcgggctcaaccaatacgtggttgggtcacagcttccatgcgagcccgaaccggatgtaacagtgctcacttccatgctcaccgacccctcccacattacagcagagacggctaagcgtaggctggccagggggtctcccccttccttggcccgctcttcagctagtcagttgtctgcgccttctctgaaggcgacatgcaccacccatcatgactccccagacgccgacctcatcgaggccaacctcctgtggcggcaggagatgggcgggaacatcacccgcgtggagtcagagaacaaagtagtgattctggactctttcgacccgcttcgagcggaggaggatgagagggaagtatccgttccggcggagatcctgcggaaaaccaggaaattcccctcagcgttgcccatatgggcacgcccggactacaacccaccactgttagagtcttggagagacccggactatgtccctccggtggtacacgggtgcccgttaccgcccaccaaggcccctccaataccacctccacggagaaagagaacagttatcctgacagaatccactgtgtcttctgccttggcggagcttgctacaaagaccttcggcagctccggatcgtcggccgtcgacagcggcacagcgactgcccctcctgatgggccctccgacgacggtgatgcaggatcggacgctgagtcatactcctccatgcccccccttgagggggagccgggggatcccgatctcagcgacgggtcttggtccactgtgagcgaggaggccagtgaggacgtcgtctgctgctcaatgtcctacacatggacaggtgccttaatcacgccatgcgccgcggaggagagcaagctgcccatcaatgcgttgagcaactctttgctgcgccaccacaacatggtctatgctacaacatcccgcagcgcaagccagcggcagaaaaaggtcacctttgacagactgcaagtcctggacgaccactaccgggacgtgctcaaggagatgaaggcgaaggcgtccacagttaaggccaaacttctatccgtagaagaggcctgcaagctgacgcccccacattcagccagatctaagtttggctatggggcaaaggacgtccggaacctatccagtaaggccgttaaccacatccactccgtgtggaaggacttgctggaagacactgagacaccaattgataccaccgtcatggcaaaaaatgaggtcttctgtgtccaaccagagaaaggaggccgcaagccagctcgccttatcgtgttcccagacctgggggtgcgtgtgtgcgagaaaatggccctttatgatgtggtctccactcttcctcaggccgtgatgggctcctcatacggattccaatactctcctggacagcgggtcgagttcctggtgaatgcctggaaatcaaagaagacccctatgggcttcgcatatgacacccgctgttttgactcaacagtcactgagaatgacatccgtgttgaggagtcaatttaccaatgttgtgacttggcccccgaggccagacaggccataaggtcgctcacagagcggctttatatcgggggccccctgactaattcaaaagggcagaactgcggttatcgccggtgccgcgccagcggcgtgctgacgactagctgcggtaacaccctcacatgttacttgaaggcctctgcagcctgtcgagctgcaaagctccaggactgcacgatgctcgtgtgcggagacgacctcgtcgttatctgtgagagcgcggggacccaggaggacgcggcgagcctgcgagtcttcacggaggctatgaccaggtactctgccccccccggggacccgccccaaccggaatacgacttggagctgataacatcatgctcctccaacgtgtcggtcgcgcacgatgcatctggcaagcgggtgtactatctcacccgcgaccccaccacccctcttgctcgggctgcgtgggagacagctagacacactccagtcaactcctggctaggcaacatcatcatgtatgcgcccaccttatgggcaaggatgattctgatgacccacttcttctcaatccttctagcccaggagcaacttgagaaagccctagattgccagatctacggggccacttactccattgagccacttgacctacctcagatcattcaacgactccatggtcttagcgcattttcactccatagttactctccaggtgagatcaatagggtggcttcatgcctcaggaaacttggggtaccgcccttgcgagtctggagacatcgggccagaagtgtccgcgctaagctactgtcccagggggggagggccgccacgtgtggcaagtacctcttcaactgggccgtacggaccaagctcaaactcactccaattccggctgcgtcccggttggacttgtccggctggttcgtcgccggttacagcgggggagacatatatcacagcctgtctcgcgcccgaccccgctggttcatgtggtgcctactcctactttccgtaggggtaggcatctacctgctccccaaccggtgaacggggagctaaacactccaggccaataggccattcctttttttttttttttttttttttttctttttttttccttttttttttttttttttttttttccttttctttcttctttggtggctccatcttagccctagtcacggctagctgtgaaaggtccgtgagccgcatgactgcagagagtgctgatactggcctctctgcagatcatgt

SEQ ID NO: 8 - protein, 3010 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNSSIVYEAADMIMHTPGCVPCVREDNSSRCWVALTPTLAARNSSVPTTTIRRHVDLLVGAAAFCSAMYVGDLCGSVFLISQLFTFSPRRYETVQDCNCSLYPGHVSGHRMAWDMMMNWSPTTALVVSQLLRIPQAVVDMVAGAHWGVLAGLAYYSMVGNWAKVLIVMLLFAGVDGNTRVSGGEAAKNTMGFASLFVSGPSQKIQLINTNGSWHINRTALNCDDSLHTGFLAALFYAHKFNSSGCSGRMASCRPIDEFAQGWGPITHGVPDNLDQRPYCWHYAPRPCGIVPASQVCGPVYCFTPSPVVVGTTDRFGAPTYSWGENETDVLLLNNTRPPQGNWFGCTWMNSTGFTKTCGGPPCNIGGVGNNTLTCPTDCFRKHPEATYTKCGSGPWLTPRCMVDYPYRLWHYPCTVNFTIFKVRMYVGGVEHRLDAACNWTRGERCNVEDRDRSELSPLLLSTTEWQILPCSFTTLPALSTGLIHLHQNIVDVQYLYGVGSVVVSVVIRWEYVVLLFLLLADARVCACLWMMLLIAQAEAALENLVVLNAASVAGAHGTLSFLVFFCAAWYIKGKLVPGAAYAFYGVWPLLLLLLALPPRAYAMDREMAASCGGAVFIGLALLTLSPHYKAFLARLIWWLQYLITRVEALLQVWVPPLNVRGGRDAIILLTCAVHPELIFEITKILLAIIGPLMVLQAGLTRVPYFVRAQGLIRVCMLVRKVAGGHYVQMALMRLAALTGTYVYNHLTPLRDWAHAGLRDLVVAVEPVVFSDMETKIITWGADTAACGDIILGLPVSARRGREILLGPADSYEGQGWRLLAPITAYSQQTRGLLGCIITSLTGRDRNQVEGEVQVVSTATQSFLATCINGVCWTVYHGAGSKTLAGPKGPITQMYTNVDQDLVGWRAPPGARSLTPCTCGSSDLYLVTRHADVIPVRRRGDNRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTSILGIGTVLDQAETAGARLVVLATATPPGSVTVPHPNIEEVALSNTGEIPFYGKAFPIEAIKGGRHLIFCHSKKKCDELATKLSALGINAVAYYRGLDVSVIPTSGDVVVVATDALMTGYTGDFDSVIDCNTCVTQTVDFSLDPTFTIETTTVPQDAVSRSQRRGRTGRGRGGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWECLIRLKPTLHGPTPLLYRLGAVQNEVTLTHPITKFIMACMSADLEVVTSTWVLVGGVLAALAAYCLTTGSVVIVGRIILSGKPAVIPDREVLYQEFDEMEECASHLPYIEQGMQLAEQFKQKALGLLQAATKQAEAAAPVVESKWQALEAFWAKHMWNFISGIQYLAGLSTLPGNPPIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPRLPGVPFLSCQRGYKGVWRGDGVMHTTCPCGAQITGHVKNGSMRIVGPKTCSNTWHGTFPINAYTTGSCTPSPAPNYSRALWRVAAEEYVEVTRVGDFHYVTGMTTDNIKCPCQVPAPEFFTELDGVRLHRYAPACKPLLRDEVTFQVGLNQYVVGSQLPCEPEPDVTVLTSMLTDPSHITAETAKRRLARGSPPSLARSSASQLSAPSLKATCTTHHDSPDADLIEANLLWRQEMGGNITRVESENKVVILDSFDPLRAEEDEREVSVPAEILRKTRKFPSALPIWARPDYNPPLLESWRDPDYVPPVVHGCPLPPTKAPPIPPPRRKRTVILTESTVSSALAELATKTFGSSGSSAVDSGTATAPPDGPSDDGDAGSDAESYSSMPPLEGEPGDPDLSDGSWSTVSEEASEDVVCCSMSYTWTGALITPCAAEESKLPINALSNSLLRHHNMVYATTSRSASQRQKKVTFDRLQVLDDHYRDVLKEMKAKASTVKAKLLSVEEACKLTPPHSARSKFGYGAKDVRNLSSKAVNHIHSVWKDLLEDTETPIDTTVMAKNEVFCVQPEKGGRKPARLIVFPDLGVRVCEKMALYDVVSTLPQAVMGSSYGFQYSPGQRVEFLVNAWKSKKTPMGFAYDTRCFDSTVTENDIRVEESIYQCCDLAPEARQAIRSLTERLYIGGPLTNSKGQNCGYRRCRASGVLTTSCGNTLTCYLKASAACRAAKLQDCTMLVCGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGATYSIEPLDLPQIIQRLHGLSAFSLHSYSPGEINRVASCLRKLGVPPLRVWRHRARSVRAKLLSQGGRAATCGKYLFNWAVRTKLKLTPIPAASRLDLSGWFVAGYSGGDIYHSLSRARPRWFMWCLLLLSVGVGIYLLPNR

SEQ ID NO: 9 - DNA, 9587 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggattaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgactaggaagacttccgagcggtcgcagcctcgtggaaggcgacaacctatccccaaggctcgccagcccgagggcagggcctgggctcagcccggatacccttggcccctctacggcaatgagggtctggggtgggcaggatggctcctgtcaccccgtggctctcggcctagttggggccctacggacccccggcgtaggtcgcgtaatttgggtaaggtcatcgataccctcacatgcggcttcgccgacctcatggggtacatcccgctcgtcggcgcccccctaggaggcgctgccagggccctggcgcatggcgtccgggttctggaggacggcgtgaactatgcaacagggaatttgcccggttgctctttctctatcttcctcttggctttgctgtcctgtttgaccatcccagcttccgcttacgaagtgcgcaacgtatccggggtgtaccatgtcacgaacgactgctccaactcaagtattgtgtatgaggcagcggacatgatcatgcacacccccgggtgcgtgccctgcgttcgggaggacaattcctcccgttgctgggtagcgctcactcccacgcttgcggccaggaacagcagcgtccccaccacgacaatacgacgccacgtcgacttgctcgttggggcagctgctttctgttccgctatgtacgtgggagacctctgcggatctgttttcctcatttcccagctgttcaccttctcacctcgccggtatgagacggtgcaagactgcaactgctcgctctatcccggccacgtatcaggtcaccgcatggcttgggatatgatgatgaactggtcacctacaacagccctggtggtgtcgcagttgctccggatcccacaagccgtcgtggacatggtggcgggggcccactggggagtcctggcgggccttgcctactattccatggtggggaactgggctaaggtcttgattgtgatgctactttttgccggcgtcgacgggaacactcgtgtgtcagggggggaagcagccaagaacaccatgggctttgcatccctctttgtgtctggcccgtcgcagaaaatccaacttattaacaccaatggcagctggcacatcaacaggactgctctgaattgcgatgactccctccacaccgggttccttgctgcgctgttttacgcgcacaagttcaactcgtccggatgctcggggcgcatggccagctgccgccccattgacgagtttgcccaggggtggggtcccatcactcacggtgtgcctgacaacctggaccagaggccttactgctggcactacgcgcctcggccgtgcggtatcgtacccgcgtcgcaggtgtgtggcccagtgtattgcttcaccccgagccctgtcgtggtggggacgaccgatcgtttcggcgcccccacgtacagctggggggagaatgagacggacgtgctactccttaacaacacgcgaccgccacaaggcaactggttcggttgcacatggatgaacagcaccgggttcaccaagacgtgcgggggccccccgtgcaacattggaggggtcggcaacaacaccttgacctgtcctacggactgcttccggaagcaccccgaggccacttacaccaaatgcggctcgggcccttggttgacacctaggtgcatggttgactacccatacagactctggcactacccttgcactgtcaattttaccatcttcaaggtcaggatgtatgtagggggtgtggagcacaggctcgacgccgcgtgcaattggacccgaggagagcgttgcaatgtggaggacagggatagatcagagcttagcccactgctactgtccacaacagagtggcagatactgccctgttccttcaccaccctaccggctctgtccactggtttgatccacctccaccagaacatcgtggacgtgcaatacctgtacggtgtggggtcagtggttgtctccgttgtaatcagatgggagtatgtcgtgctgctcttccttctcctggcggacgcacgcgtctgcgcctgcttgtggatgatgctgctgatagctcaggctgaggccgccttagagaacttggtggtcctcaatgcggcatctgtagctggagcgcatggcactctctccttccttgtgttcttctgtgccgcctggtacatcaagggcaagctggtccctggagcggcatatgctttctatggcgtgtggccgctgctcctgctcctgctggcattaccaccacgagcatacgccatggaccgggagatggctgcatcatgcggaggcgcggtcttcataggtctggcactcctgaccttgtcaccacactataaagcgttcctcgctaggctcatttggtggttgcagtatcttatcaccagggtcgaggcgctactgcaagtgtgggtcccccctcttaacgttcgggggggccgcgatgccatcatcctcctcacgtgcgcggtccatccagagctaatctttgaaatcaccaaaatcttgctcgccataattggtccgctcatggtgctccaggctggcttaaccagagtgccgtacttcgtgcgcgctcaggggctcatccgtgtgtgcatgttggtgcggaaagtcgctgggggtcactacgtccagatggctctcatgaggctggccgcgctgacgggcacgtacgtttacaaccatcttactccgctgcgggactgggcccacgcgggcttgcgggaccttgtggtggcagttgagcccgtcgtcttctctgacatggagaccaagatcatcacctggggggcagacaccgcggcgtgtggggacatcatcttgggtctacccgtctccgcccgaagggggagggagatacttttgggaccggccgacagttatgaagggcagggatggcggcttcttgcgcctatcacggcctattcccaacagacgcggggcctactcggttgcatcatcactagtctcacaggccgggacaggaaccaggtcgagggggaggttcaggtggtttccaccgcaacgcagtccttcttggcgacctgcatcaatggtgtgtgttggactgtctaccatggtgccggctcaaagaccctagccggcccaaagggtccgatcacccaaatgtacaccaatgtagaccaggacctcgtcggctggcgggcgcccctcggggcgcgttccttaacaccatgcacctgcggcagctcggacctctacttggtcacgaggcatgctgatgtcattccggtgcgccggcggggcgataacaggggcagcctgctttcccccagacctgtctcttacttaaagggctcctcgggtggtccactgctctgcccctcagggcacgctgtgggcatcttccgggccgccgtgtgcaccaggggggttgcgaaggcggtggactttgtgcccgttgagtccatggaaactaccatgcggtctccggtcttcacggacaattcatctcccccggccgtaccgcagacattccaagtggcccatctacacgctcccactggcagcggtaagagcactaaggtgccggctgcttatgcagcccaagggtacaaggtactcgtcctgaacccatccgttgccgccaccttaggttttggggcgtatatgtctaaagcacatggtgttgaccccaacatcagaactggggtaaggaccatcaccacaggcgctcccattacgtattccacttatggcaagttccttgccgacggtggttgctctgggggcgcctatgacatcataatatgtgatgagtgccactcaactgactcgacttccatcttgggcatcggcactgtcttggaccaagcggagacggctggagcgcggctcgtcgtgctcgccaccgctacacctccgggatcagtcaccgtgccacatcccaatatcgaggaggtggctttgtccaacactggagagatccccttttatggtaaggcctttcccatcgaggccatcaagggggggaggcatctcattttctgccactccaagaagaaatgtgacgagctcgccacaaagctgtcggcccttggaatcaacgctgtagcgtactaccggggtcttgatgtgtccgtcataccgacaagcggagatgtcgttgtcgtggcaacagacgctctgatgacgggctacactggcgactttgactcagtgatcgactgtaacacatgtgtcacccagacagtcgacttcagcttggaccctaccttcaccattgagacgacgaccgtgccccaagacgcggtatcgcgctcgcaacggcgaggcaggactggtaggggcagggggggcatatacaggtttgtgactccaggggaacggccctcgggcatgttcgattcttcggtcctgtgcgagtgctatgacgcgggctgtgcttggtacgagctcacgcccgccgagacctcggttaggttgcgggcttacctaaatacaccagggttgcccgtctgccaggaccatctggagttctgggagagcgtcttcacaggcctcacccacatagatgcccatttcttatcccagaccaagcaggcaggagacaacttcccctacctggtagcgtaccaggctacggtgtgcgccagggcccaggccccaccaccatcgtgggaccaaatgtggaagtgtctcatacggctaaagcctacactgcacgggccaacacccctgctgtacaggctaggagccgtccaaaacgaagtcactctcacacaccccataaccaaattcatcatggcatgcatgtcggctgacctagaggtcgtcacgagcacctgggtgctagtaggcggggtccttgcagctctggccgcgtactgcctgacaacgggcagcgtggtcattgtgggtaggatcatcttgtctgggaagccggctgtcattcccgatagggaagtgctctaccaggagttcgatgaaatggaagagtgcgcctcgcacctcccttacattgaacagggaatgcagctcgccgagcaattcaagcagaaggcgctcgggttgctgcaagcagccaccaagcaagcggaggctgctgctcccgtggtggagtccaagtggcaagcccttgaggccttttgggcgaagcacatgtggaatttcatcagcgggatacagtacttagcaggcttgtccactctgcctgggaaccccccgatagcttcactgatggcattcacagcctctatcaccagcccgctcaccacccagcataccctcttgtttaacatcttgggggggtgggtggcagcccaactcgccccccccagcgctgcttcagctttcgtgggcgccgggatcgctggcgcggctgttggcagcataggccttgggaaggtgcttgtggacatcctggcgggctatggggcgggggtggcaggcgcgctcgtggcctttaaggtcatgagcggcgaaatgccctccaccgaggacctagtcaacttgctccctgccatcctctcccccggtgccctggttgtcggggtcgtgtgcgcggcaatactgcgccggcacgtgggcccgggggagggggctgtgcagtggatgaaccggctgatagcgttcgcttcgcggggtaaccatgtttcccccacgcactatgtgcctgagagcgacgcggccgcgcgtgtcactcagatcctctccagccttaccatcacccagctgttgaagagactccaccagtggatcaatgaggactgctccacgccatgctccggctcgtggctcagggatgtttgggactggatatgcacggtgttgactgacttcaagacctggctccagtccaagctcctgccacggttgccgggggtccctttcctttcatgccaacgtggatataagggagtctggcgaggagatggcgtcatgcacaccacttgcccatgtggcgcacaaatcaccggacatgtcaaaaacggttccatgaggatcgttgggcctaaaacctgtagcaacacgtggcacggaacattccccatcaacgcgtacaccacgggttcctgcacaccctccccggcgccaaactattccagggcgttgtggcgggtagctgctgaggagtacgtggaggtcacgcgggtgggggatttccactacgtgacgggcatgaccactgacaacataaaatgcccatgccaggttccggcccccgaatttttcacagagttggacggggtgcggctgcacaggtacgctccggcgtgcaaacctctcttacgggatgaggtcacattccaggtcgggctcaaccaatacgtggttgggtcacagcttccatgcgagcccgaaccggatgtaacagtgctcacttccatgctcaccgacccctcccacattacagcagagacggctaagcgtaggctggccagggggtctcccccttccttggcccgctcttcagctagtcagttgtctgcgccttctctgaaggcgacatgcaccacccatcatgactccccagacgccgacctcatcgaggccaacctcctgtggcggcaggagatgggcgggaacatcacccgcgtggagtcagagaacaaagtagtgattctggactctttcgacccgcttcgagcggaggaggatgagagggaagtatccgttccggcggagatcctgcggaaaaccaggaaattcccctcagcgttgcccatatgggcacgcccggactacaacccaccactgttagagtcttggagagacccggactatgtccctccggtggtacacgggtgcccgttaccgcccaccaaggcccctccaataccacctccacggagaaagagaacagttatcctgacagaatccactgtgtcttctgccttggcggagcttgctacaaagaccttcggcagctccggatcgtcggccgtcgacagcggcacagcgactgcccctcctgatgggccctccgacgacggtgatgcaggatcggacgctgagtcatactcctccatgcccccccttgagggggagccgggggatcccgatctcagcgacgggtcttggtccactgtgagcgaggaggccagtgaggacgtcgtctgctgctcaatgtcctacacatggacaggtgccttaatcacgccatgcgccgcggaggagagcaagctgcccatcaatgcgttgagcaactctttgctgcgccaccacaacatggtctatgctacaacatcccgcagcgcaagccagcggcagaaaaaggtcacctttgacagactgcaagtcctggacgaccactaccgggacgtgctcaaggagatgaaggcgaaggcgtccacagttaaggccaaacttctatccgtagaagaggcctgcaagctgacgcccccacattcagccagatctaagtttggctatggggcaaaggacgtccggaacctatccagtaaggccgttaaccacatccactccgtgtggaaggacttgctggaagacactgagacaccaattgataccaccgtcatggcaaaaaatgaggtcttctgtgtccaaccagagaaaggaggccgcaagccagctcgccttatcgtgttcccagacctgggggtgcgtgtgtgcgagaaaatggccctttatgatgtggtctccactcttcctcaggccgtgatgggctcctcatacggattccaatactctcctggacagcgggtcgagttcctggtgaatgcctggaaatcaaagaagacccctatgggcttcgcatatgacacccgctgttttgactcaacagtcactgagaatgacatccgtgttgaggagtcaatttaccaatgttgtgacttggcccccgaggccagacaggccataaggtcgctcacagagcggctttatatcgggggccccctgactaattcaaaagggcagaactgcggttatcgccggtgccgcgccagcggcgtgctgacgactagctgcggtaacaccctcacatgttacttgaaggcctctgcagcctgtcgagctgcaaagctccaggactgcacgatgctcgtgtgcggagacgacctcgtcgttatctgtgagagcgcggggacccaggaggacgcggcgagcctgcgagtcttcacggaggctatgaccaggtactctgccccccccggggacccgccccaaccggaatacgacttggagctgataacatcatgctcctccaacgtgtcggtcgcgcacgatgcatctggcaagcgggtgtactatctcacccgcgaccccaccacccctcttgctcgggctgcgtgggagacagctagacacactccagtcaactcctggctaggcaacatcatcatgtatgcgcccaccttatgggcaaggatgattctgatgacccacttcttctcaatccttctagcccaggagcaacttgagaaagccctagattgccagatctacggggccacttactccattgagccacttgacctacctcagatcattcaacgactccatggtcttagcgcattttcactccatagttactctccaggtgagatcaatagggtggcttcatgcctcaggaaacttggggtaccgcccttgcgagtctggagacatcgggccagaagtgtccgcgctaagctactgtcccagggggggagggccgccacgtgtggcaagtacctcttcaactgggccgtacggaccaagctcaaactcactccaattccggctgcgtcccggttggacttgtccggctggttcgtcgccggttacagcgggggagacatatatcacagcctgtctcgcgcccgaccccgctggttcatgtggtgcctactcctactttccgtaggggtaggcatctacctgctccccaaccggtgaacggggagctaaacactccaggccaataggccattcctttttttttttttttttttttttttctttttttttccttttttttttttttttttttttttccttttctttcttctttggtggctccatcttagccctagtcacggctagctgtgaaaggtccgtgagccgcatgactgcagagagtgctgatactggcctctctgcagatcatgt

SEQ ID NO: 10 - protein, 3010 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNSSIVYEAADMIMHTPGCVPCVREDNSSRCWVALTPTLAARNSSVPTTTIRRHVDLLVGAAAFCSAMYVGDLCGSVFLISQLFTFSPRRYETVQDCNCSLYPGHVSGHRMAWDMMMNWSPTTALVVSQLLRIPQAVVDMVAGAHWGVLAGLAYYSMVGNWAKVLIVMLLFAGVDGNTRVSGGEAAKNTMGFASLFVSGPSQKIQLINTNGSWHINRTALNCDDSLHTGFLAALFYAHKFNSSGCSGRMASCRPIDEFAQGWGPITHGVPDNLDQRPYCWHYAPRPCGIVPASQVCGPVYCFTPSPVVVGTTDRFGAPTYSWGENETDVLLLNNTRPPQGNWFGCTWMNSTGFTKTCGGPPCNIGGVGNNTLTCPTDCFRKHPEATYTKCGSGPWLTPRCMVDYPYRLWHYPCTVNFTIFKVRMYVGGVEHRLDAACNWTRGERCNVEDRDRSELSPLLLSTTEWQILPCSFTTLPALSTGLIHLHQNIVDVQYLYGVGSVVVSVVIRWEYVVLLFLLLADARVCACLWMMLLIAQAEAALENLVVLNAASVAGAHGTLSFLVFFCAAWYIKGKLVPGAAYAFYGVWPLLLLLLALPPRAYAMDREMAASCGGAVFIGLALLTLSPHYKAFLARLIWWLQYLITRVEALLQVWVPPLNVRGGRDAIILLTCAVHPELIFEITKILLAIIGPLMVLQAGLTRVPYFVRAQGLIRVCMLVRKVAGGHYVQMALMRLAALTGTYVYNHLTPLRDWAHAGLRDLVVAVEPVVFSDMETKIITWGADTAACGDIILGLPVSARRGREILLGPADSYEGQGWRLLAPITAYSQQTRGLLGCIITSLTGRDRNQVEGEVQVVSTATQSFLATCINGVCWTVYHGAGSKTLAGPKGPITQMYTNVDQDLVGWRAPLGARSLTPCTCGSSDLYLVTRHADVIPVRRRGDNRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTSILGIGTVLDQAETAGARLVVLATATPPGSVTVPHPNIEEVALSNTGEIPFYGKAFPIEAIKGGRHLIFCHSKKKCDELATKLSALGINAVAYYRGLDVSVIPTSGDVVVVATDALMTGYTGDFDSVIDCNTCVTQTVDFSLDPTFTIETTTVPQDAVSRSQRRGRTGRGRGGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKFIMACMSADLEVVTSTWVLVGGVLAALAAYCLTTGSVVIVGRIILSGKPAVIPDREVLYQEFDEMEECASHLPYIEQGMQLAEQFKQKALGLLQAATKQAEAAAPVVESKWQALEAFWAKHMWNFISGIQYLAGLSTLPGNPPIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPRLPGVPFLSCQRGYKGVWRGDGVMHTTCPCGAQITGHVKNGSMRIVGPKTCSNTWHGTFPINAYTTGSCTPSPAPNYSRALWRVAAEEYVEVTRVGDFHYVTGMTTDNIKCPCQVPAPEFFTELDGVRLHRYAPACKPLLRDEVTFQVGLNQYVVGSQLPCEPEPDVTVLTSMLTDPSHITAETAKRRLARGSPPSLARSSASQLSAPSLKATCTTHHDSPDADLIEANLLWRQEMGGNITRVESENKVVILDSFDPLRAEEDEREVSVPAEILRKTRKFPSALPIWARPDYNPPLLESWRDPDYVPPVVHGCPLPPTKAPPIPPPRRKRTVILTESTVSSALAELATKTFGSSGSSAVDSGTATAPPDGPSDDGDAGSDAESYSSMPPLEGEPGDPDLSDGSWSTVSEEASEDVVCCSMSYTWTGALITPCAAEESKLPINALSNSLLRHHNMVYATTSRSASQRQKKVTFDRLQVLDDHYRDVLKEMKAKASTVKAKLLSVEEACKLTPPHSARSKFGYGAKDVRNLSSKAVNHIHSVWKDLLEDTETPIDTTVMAKNEVFCVQPEKGGRKPARLIVFPDLGVRVCEKMALYDVVSTLPQAVMGSSYGFQYSPGQRVEFLVNAWKSKKTPMGFAYDTRCFDSTVTENDIRVEESIYQCCDLAPEARQAIRSLTERLYIGGPLTNSKGQNCGYRRCRASGVLTTSCGNTLTCYLKASAACRAAKLQDCTMLVCGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGATYSIEPLDLPQIIQRLHGLSAFSLHSYSPGEINRVASCLRKLGVPPLRVWRHRARSVRAKLLSQGGRAATCGKYLFNWAVRTKLKLTPIPAASRLDLSGWFVAGYSGGDIYHSLSRARPRWFMWCLLLLSVGVGIYLLPNR

SEQ ID NO: 11 - DNA, 9587 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggtccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgactaggaagacttccgagcggtcgcaacctcgtggaaggcgacaacctatccccaaggctcgccggcccgagggcagggcctgggctcagcccgggtacccctggcccctctatggcaatgagggtctggggtgggcaggatggctcctgtctccccgtggctcccggcctagttggggccccacggacccccggcgtaggtcgcgtaatttgggtaaagtcatcgataccctcacatgcggcttcgccgacctcatggggtacattccgctcgtcggcgctcctctaggaggcgctgccagggccctggcgcatggcgtccgggttctggaggacggcgtgaactacgcaacagggaatctgcccggttgctctttctctatcttcctcttggctttgctgtcctgtttgaccaccccagcttccgcttatgaagtgcgcaacgtgtccggagtgtaccatgtcacgaacgactgctccaactcaagtattgtgtatgaggcagcggacatgatcatgcacacccctgggtgcgtaccctgcgtccgggagaacaactcctcccgctgctgggtagcgctcactcccacgctcgcggccaggaacagcagcatccccactacgacaatacggcgccatgtcgatttgctcgtcggggcggctgctctctgctccgctatgtatgtgggggatctctgcggatctgttttcctcgtctcccagctgttcaccttttcacctcgccggtacgagacggtacaggactgcaattgctcactctatcccggccacgtatcaggtcaccgcatggcctgggatatgatgatgaactggtcgcccacgacagccctagtggtatcgcagttactccggattccacaatccgtcgtggacatggtggcgggggcccactggggagtcctggcgggccttgcctactattccatggtggggaactgggctaaggtcttgattgtgatgctactctttgctggcgttgacgggagcacccgcgtaacagggggaacgacagcctacaacgtcaggggccttacgtccctctttacaccagggccgtctcagaaaatccagctcataaattctaacggcagctggcacatcaacagaactgccttgaattgcaatgactcccttcaaactgggttccttgctgcgttgttctacacacatagtttcaacgcgtccggatgcccagagcgcatggccagctgccgcccaattgacaagtttgctcaggggtggggtcccatcacccatgttgtgcctaacatctcagaccagaggccctattgctggcactacgcacctcgaccgtgtggtatcgtacctgcgtcgcaggtgtgtggtccggtgtattgcttcaccccgagccctgttgtggtgggaacgaccgatcgtttcggcgtccccacatatacctggggggagaatgagacggacgtgctgctcctcaacaacacgcggccgccgcaaggcaactggttcggctgtacatggatgaatagcaccgggttcaccaagacatgcgggggccccccgtgtgacatcgggggggccggcaatagcaccttgacctgccctacggattgcttccggaagcaccccgaggctacctacaccaagtgcggctcggggccttggttgacacccaggtgtatagttgactacccatacaggctttggcactacccctgcactgtcaactttaccatcttcaaggtcaggatgtatgtggggggcgtggagcataggctcaacgccgcgtgcaattggactcgaggagagcgttgcaacctggaggacagggacaggtcggagctcagcccgctgctgctgtctacaacagagtggcagatactgccctgttccttcaccaccctaccggctctgtccactggtttgattcacctccatcagaacatcgtggacgtgcaatacctgtatggtgtagggtcagcagttgtctccattgtgatcaaatgggagtatgtcctgctgctcttccttctcctggcagatgcgcgcgtctgcgcctgcttatggatgatgctgctgatagcccaggccgaggctgcattggagaacctggtgatcctcaatgcggcgtccgtggccggagcacacggtatcctctccttccttgtgttcttctgtgctgcctggtacatcaagggcaagctggtccctggggcggcatatgccctctatggcgtgtggccgctgctcctgctcctgctggcgttgccaccacgggcgtacgccatggaccgggagatggctgcatcgtgcggaggcgcggttttcataggtctggtactcttgaccctgtcaccacactataaagcattcctcgccaggctcatatggtggttgcaatattttaccaccagggccgaggcgcacttgcaagtgtgggtttcccccctcaacgttcgggggggccgcgatgccatcatcctcctcacgtgcgtggtccacccagagctgatttttgacatcaccaaaatcttgctcgccatatttggtccgctcatggtgctccaggctggcctaactagagtgccgtacttcgtgcgcgctcaagggctcatccgtgcgtgcgtgttggtgcggaaggtcgctgggggtcattatgtccaaatggctctcgtgaagctggccgctctgacgggcacgtacgtttatgaccatcttactccgctgcgggactgggcccacgcgagcctgcaagaccttgtggtagcggtcgagcccgtcgtcttctctgacatggagaccaagatcatcacctggggggcagacaccgcagcgtgtggagatatcatttcgggcctacccgtctccgctcgaagggggagggagatacttctggggccggccgatagttttgaagagcaagggtggcggctcctcgcgcccatcacggcctactcccaacagacgcggggcttgcttggctgcatcatcactagtctcacaggtcgggacaagaaccaggtcgagggggaggtgcaggtggtttccaccgcgacgcaatctttcctggcgacctgtgtcaatggtgtgtgctggtctgtttaccacggcgccggctcaaagaccttagccggcccaaaggggccagtcacccaaatgtacaccaatgtagatctggacctcgtcggctggcaggcgccccccgggacgcgctccctgacaccgtgcacctgcggcagctcggacctttacttggtcacgagacatgctgacgtcattccggtgcgccggcggggcgacagcagggggagcctactttcccccaggcccgtctcctacttgaagggctcctcgggcggtccactgctctgcccctcggggcacgctgtgggcatcttccgggctgctgtgtgcacccggggggttgccaaggcggtggactttatacccgttgagtctatggaaaccaccatgcggtctccggtctacacggacaactcatctcccccggctgtaccgcagactttccaagtggcccacctgcacgctcccactggcagcggtaagagcactaaggtgccggctgcatatgcagcccaagggtacaaggtgctcgtcctgaacccgtccgttgccgctaccctaggttttggggcgtacatgtccaaagcacatggtgttgaccctaacatcaggactggggtaaggaccatcaccacgggcgctcccatcacgtactccacctacggcaagttccttgccgacggtggttgctctgggggcgcctatgacatcataatatgtgatgagtgtcactcaactgactcgacttctatcttgggcattggtacggtcttggaccaagcggagacggctggagcgcggctcgtcgtgctcgccaccgctacacctccgggatcggttactgtgccacatcctaacatcgaggaggtggccttgtccaacactggagagattcccttctatggcaaagccatccccatcgagaccatcaaggggggaaggcatctcatcttctgccattctaagaagaagtgtgatgaactcgctgcaaagctgtcggcccttggggtcaacgctgtagcgtactaccggggtcttgatgtgtccgtcataccgacgagcggagacgtcgttgtcgtggcaacagacgctctaatgacgggctacaccggtgactttgactcagtgatcgactgtaacacatgtgtcacccagacagtcgatttcagcttggaccccaccttcaccattgacaccacgactgtgccccaggacgccgtgtcgcgctcacagcggcgaggtaggactggtaggggcagagggggcatatataggtttgtgactccaggggaacggccctcgggcatgttcgattcctcggtcctgtgtgaatgctatgacgcgggctgtgcttggtacgagctcacgcccgccgagacctcagttaggctgcgggcttacctaaatacaccagggttgcccgtttgccaggaccatttggagttctgggagagcgtcttcacaggcctcacccacatagatgctcacttcttgtcccagactaagcaggcaggagacaacttcccctacctggtggcataccaggctacagtgtgcgccagggcccaggctccacctccgtcgtgggatcagatgtggaagtgtctcatacggctaaagcctacgctgcacgggccaacgcccctgttgtataggctaggagccgtccaaaatgaggtcaccctcacacaccccataaccaaattcatcatggcatgcatgtcggctgacctggaggtcgtcacgagcacctgggtgctagtgggcggcgtcctcgcagcgctggccgcgtactgcctgacaacgggcagcgtggttatcgtgggcaggatcatcttgtccgggaggccggctatcattcccgacagggaagttctctaccaggagttcgatgaaatggaagagtgcgcctcacaccttccctacatcgaacaaggaatgcagctcgccgagcaattcaaacagaaggcgctcgggctgctgcagacagccaccaagcaagcagaggctgctgctcccgtggtgcagtccaagtggcaagcccttgaggccttctgggcgaagcacatgtggaacttcatcagcgggatacagtatttggcaggcttatccactctgcctgggaaccccgcgatagcatcactgatggcattcacagcctccatcaccagcccgctcaccacccaatataccctcctgtttaacatcttggggggatgggtggccgcccaactcgccccccctagcgccgcttcagcttttgtgggcgccggtatcgctggcgcggctgttggcagcataggccttgggaaggtgcttgtggacatcttggcgggttatggagcaggggtggcaggcgcacttgtggcctttaaggtcatgagcggcgaagtgccttccactgaggacctggtcaacttactccctgccatcctctctcctggtgccctggtcgtcggggtcgtgtgcgcagcaatactgcgtcggcatgtgggcccaggggagggggctgtgcagtggatgaaccggctgatagcgttcgcttcgcggggtaaccacgtctcccccacgcactatgtgcctgagagcgacgctgcggcgcgtgtcacccagatcctctccagccttaccatcactcagctgttaaagaggctccaccagtggatcaatgaggactgctccacgccatgctccggctcgtggctcagggatgtttgggactggatatgcacggtgttgagtgacttcaagacctggctccagtccaagctcctgccgcggctgccgggagtcccttttttctcatgtcaacgtggatacaggggagtctggcgaggagatggcgtcatgcaaaccatctgcccatgtggagcacaaatcagcggacatgtcaagaacggttccatgaggatcgttgggcctaaaacctgtagcaacacgtggcatggaacattccccatcaacgcgtacaccacgggcccctgcacaccctccccagcgcccaactattccagggcgctgtggcgggtgactgctgaggagtacgtggaggtcacgcgggtgggggatttccactacgtgacgggcatgaccactgacaacgtaaaatgcccatgccaggttccggcccccgaattcttcacggaattggacggggtgcggctgcacaggtacgctccggcgtgcaaacctctcctacgggatgaggtcacattccaggtcgggctcaaccacttcccggttgggtctcaactcccatgtgagcccgaaccggatgtaacagtggtcacttccatgctgaccgacccctcccacattacggcagaaacggctaggcgtaggctggccagagggtctcccccttccttggccagctcctcagctagtcagttgtctgcgccctccttgaaggcgacatgcactacccatcatgactccccggacgccgacctcatcgaggccaacctcctgtggcggcaggagatgggcggaaacatcacccgtgtggagtcggagaataaggtagtaatcctagactcttttgatccgcttcgagcggaggaggatgagagggaagtatccgtagcggcggagatcctgcggaaaaccaggaagttccccccagcgatgcctatatgggcacgcccggattacaacccaccactcctagagtcttggaagaacccagattacgtccctccggtggtgcacgggtgtccattgccacctaccaaggcccctccaataccacctccacggagaaagaggacggttgtcctgacagaatccaccgtgtcttctgccttggcggagcttgctaccaagaccttcggcagctccggatcatcagccgtcgacagcggcacagcgagcgccccccctgatcagccttccgatgacggagatgcaggatccgacgttgaatcgtactcctccatgcccccccttgagggagagccgggggaccccgatctcagcgacgggtcttggtctactgtgagcgaggaggctagtgaggacgtcgtctgttgttcgatgtcctacacatggacaggcgctttaatcacaccatgcgccgcggaggagagcaagctgcccatcaatgcgttgagcaactctttgctgcgtcaccataacatggtctatgccacaacatcccgcagcgcaggccaacggcagaaaaaggtcacctttgacagactgcaggtcctggacgaccactaccgggacgtgctcaaggagatgaaggcgaaggcgtccacagttaaggctaaacttctatcagtagaagaagcctgcaagctgacgcccccacattcggccagatctaaatttggctatggggcaaaggacgtccggaacctatccagcaaggccgttaaccacatccactccgtgtggaaggacttgctggaagacactgagacaccaattgacactaccatcatggcaaaaaatgaggtcttctgtgttcaaccagagaaaggaggccgcaagccagctcgcctcatcgtattcccagacttgggggttcgtgtatgcgagaaaatggccctttatgacgtggtctccactcttcctcaggccgtgatgggctcctcatacggattccagtactctcctgggcagcgggtcgagttcctggtgaacgcctggaaatcaaagaaaaaccctatgggctttgcatatgacacccgctgttttgactcaacggtcactgagaatgacatccgtgttgaggagtcaatttaccaatgttgtgacttggcccccgaggccaggcaggccataaggtcgctcacagagcggctttatatcgggggccccctgactaattcaaaagggcagaactgcggttatcgccggtgccgcgccagcggcgtgctgacgaccagctgcggtaacaccctcacatgttacttgaaggccaccgcagcctgtcgagctgcaaagctccaggactgcacgatgctcgtgtgcggagacgaccttgtcgttatctgtgaaagcgcgggaacccaggaggatgcggcgagcctacgagtcttcacggaggctatgactaggtactctgccccccccggggacccgccccaaccggaatacgacttggagttaataacatcatgctcctccaacgtgtcggtcgcgcacgatgcatctggcaagcgggtgtactacctcacccgggaccccaccaccccccttgcgcgggctgcgtgggagacagcaagacacactccagttaattcctggctaggcaacatcatcatgtatgcgcccacattgtgggcaaggatgattctgatgacccactttttctccgtccttctagctcaggaacaacttgaaaaagccctagattgtcaaatctacggggccacttactccattgagccacttgacctacctcagatcattcaacgactccacggtcttagcgcattttcactccatagttactctccaggtgagatcaatagggtggcttcatgcctcaggaaacttggggtaccacccttgcgagtctggagacatcgggccagaagtgtccgcgctaagctactgtcccagaaggggagggctgccacttgtggcaagtacctcttcaactgggccgtacggaccaagctcaaactcactccaattccggctgcgtcccggttggacttgtccggctggttcgtcgccggttacagcgggggagacatatatcacagcctgtctcgcgcccgaccccgctggttcatgtggtgcctactcctactttccgtaggggtaggcatctacctgctccccaaccggtgaacggggagctaaacactccaggccaataggccattcctttttttttttttttttttttttttctttttttttccttttttttttttttttttttttttccttttctttcttctttggtggctccatcttagccctagtcacggctagctgtgaaaggtccgtgagccgcatgactgcagagagtgctgatactggcctctctgcagatcatgt

SEQ ID NO: 12 - protein, 3010 aa, Hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTTPASAYEVRNVSGVYHVTNDCSNSSIVYEAADMIMHTPGCVPCVRENNSSRCWVALTPTLAARNSSIPTTTIRRHVDLLVGAAALCSAMYVGDLCGSVFLVSQLFTFSPRRYETVQDCNCSLYPGHVSGHRMAWDMMMNWSPTTALVVSQLLRIPQSVVDMVAGAHWGVLAGLAYYSMVGNWAKVLIVMLLFAGVDGSTRVTGGTTAYNVRGLTSLFTPGPSQKIQLINSNGSWHINRTALNCNDSLQTGFLAALFYTHSFNASGCPERMASCRPIDKFAQGWGPITHVVPNISDQRPYCWHYAPRPCGIVPASQVCGPVYCFTPSPVVVGTTDRFGVPTYTWGENETDVLLLNNTRPPQGNWFGCTWMNSTGFTKTCGGPPCDIGGAGNSTLTCPTDCFRKHPEATYTKCGSGPWLTPRCIVDYPYRLWHYPCTVNFTIFKVRMYVGGVEHRLNAACNWTRGERCNLEDRDRSELSPLLLSTTEWQILPCSFTTLPALSTGLIHLHQNIVDVQYLYGVGSAVVSIVIKWEYVLLLFLLLADARVCACLWMMLLIAQAEAALENLVILNAASVAGAHGILSFLVFFCAAWYIKGKLVPGAAYALYGVWPLLLLLLALPPRAYAMDREMAASCGGAVFIGLVLLTLSPHYKAFLARLIWWLQYFTTRAEAHLQVWVSPLNVRGGRDAIILLTCVVHPELIFDITKILLAIFGPLMVLQAGLTRVPYFVRAQGLIRACVLVRKVAGGHYVQMALVKLAALTGTYVYDHLTPLRDWAHASLQDLVVAVEPVVFSDMETKIITWGADTAACGDIISGLPVSARRGREILLGPADSFEEQGWRLLAPITAYSQQTRGLLGCIITSLTGRDKNQVEGEVQVVSTATQSFLATCVNGVCWSVYHGAGSKTLAGPKGPVTQMYTNVDLDLVGWQAPPGTRSLTPCTCGSSDLYLVTRHADVIPVRRRGDSRGSLLSPRPVSYLKGSSGGPLLCPSGHAVGIFRAAVCTRGVAKAVDFIPVESMETTMRSPVYTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTSILGIGTVLDQAETAGARLVVLATATPPGSVTVPHPNIEEVALSNTGEIPFYGKAIPIETIKGGRHLIFCHSKKKCDELAAKLSALGVNAVAYYRGLDVSVIPTSGDVVVVATDALMTGYTGDFDSVIDCNTCVTQTVDFSLDPTFTIDTTTVPQDAVSRSQRRGRTGRGRGGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKFIMACMSADLEVVTSTWVLVGGVLAALAAYCLTTGSVVIVGRIILSGRPAIIPDREVLYQEFDEMEECASHLPYIEQGMQLAEQFKQKALGLLQTATKQAEAAAPVVQSKWQALEAFWAKHMWNFISGIQYLAGLSTLPGNPAIASLMAFTASITSPLTTQYTLLFNILGGWVAAQLAPPSAASAFVGAGIAGAAVGSIGLGKVLVDILAGYGAGVAGALVAFKVMSGEVPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLSDFKTWLQSKLLPRLPGVPFFSCQRGYRGVWRGDGVMQTICPCGAQISGHVKNGSMRIVGPKTCSNTWHGTFPINAYTTGPCTPSPAPNYSRALWRVTAEEYVEVTRVGDFHYVTGMTTDNVKCPCQVPAPEFFTELDGVRLHRYAPACKPLLRDEVTFQVGLNHFPVGSQLPCEPEPDVTVVTSMLTDPSHITAETARRRLARGSPPSLASSSASQLSAPSLKATCTTHHDSPDADLIEANLLWRQEMGGNITRVESENKVVILDSFDPLRAEEDEREVSVAAEILRKTRKFPPAMPIWARPDYNPPLLESWKNPDYVPPVVHGCPLPPTKAPPIPPPRRKRTVVLTESTVSSALAELATKTFGSSGSSAVDSGTASAPPDQPSDDGDAGSDVESYSSMPPLEGEPGDPDLSDGSWSTVSEEASEDVVCCSMSYTWTGALITPCAAEESKLPINALSNSLLRHHNMVYATTSRSAGQRQKKVTFDRLQVLDDHYRDVLKEMKAKASTVKAKLLSVEEACKLTPPHSARSKFGYGAKDVRNLSSKAVNHIHSVWKDLLEDTETPIDTTIMAKNEVFCVQPEKGGRKPARLIVFPDLGVRVCEKMALYDVVSTLPQAVMGSSYGFQYSPGQRVEFLVNAWKSKKNPMGFAYDTRCFDSTVTENDIRVEESIYQCCDLAPEARQAIRSLTERLYIGGPLTNSKGQNCGYRRCRASGVLTTSCGNTLTCYLKATAACRAAKLQDCTMLVCGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSVLLAQEQLEKALDCQIYGATYSIEPLDLPQIIQRLHGLSAFSLHSYSPGEINRVASCLRKLGVPPLRVWRHRARSVRAKLLSQKGRAATCGKYLFNWAVRTKLKLTPIPAASRLDLSGWFVAGYSGGDIYHSLSRARPRWFMWCLLLLSVGVGIYLLPNR

SEQ ID NO: 13 - DNA, 9587 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgactaggaagacttccgagcggtcgcaacctcgtggaaggcgacaacctatccccaaggctcgccagcccgagggcagggcctgggctcagcccgggtacccttggcccctctatggcaatgagggtctggggtgggcaggatggctcctgtcaccccgaggctctcggcctagttggggccccacggacccccggcgtaggtcgcgtaatctgggtaaggtcatcgatacccttacatgcggcttcgccgacctcatggggtacattccgctcgtcggcgcccccctagggggcgttgccagggccctggcgcaaggcgtccgggttctggaggacggcgtgaactacgcaacagggaatttgcccggttgctccttttctatcttcctcttggctttgctgtcttgcttgaccattccaacttccgcttacgaagtgcgcaacgtgtccggggtgtaccatgtcacgaacgactgctccaactcaagtatagtgtatgaggcagcggacatgatcatgcacacccccgggtgcgtgccctgcgtccgggagagtaatttctcccgctgctgggtggcgctcactcccacgctcgcggccaggaacagcagcatccccaccacgacaatacgacgccacgtcgatttgctcgttggggcagccgctttctgctctgctatgtacgtgggagatctctgcggatccgtcttcctcgtctctcagctgttcaccttctcacctcgccggtacgagacggtacaagactgcaactgctcactctatcccggccacgtatcaggtcaccgcatggcttgggatatgatgatgaattggtcacctacagcagccctagtggtatcgcagttactccggatcccacaagctatcgtggatatggtggcgggggcccactggggagtcctagcgggccttgcctactattccatggtggggaactgggctaaggttttgattgtgatgctactttttgccggcgttgacgggagcacccgcgtgtcgggggggcaggtagcctacaacaccaggggctttgcatccctctttacaccaggggcgagtcagaaaatccaactcataaacaccaacggcagctggcacatcaacaggactgctctgaactgcaatgactccctccacactgggttccttgccgcgctgttctacacacacaagttcaacgcgtccggatgcccagagcgcatggccagctgccgccccattgacaagttcgctcaggggtggggtcccatcactcatgtcgagcctgagatctcggaccagaggccatattgctggcactacgcaccccgaccgtgcggtatagtacccgcgtcgcaggtgtgtggtccagtgtattgcttcaccccaagccctgttgtagtggggacgactgatcgttccggcgtccccacgtacacctggggggagaatgagacggacgtgctgctccttaacaacacgcggccaccccacggcaactggttcggctgcacatggatgaatagcaccgggttcaccaagacgtgcgggggccccccgtgcaacatcgggggggtcggcaacaacaccttgacctgccccacggattgcttccggaaacaccccgaagccacttacaccaaatgcggctcgggcccttggttgacacctaggtgtatagttgactacccatacagactttggcactacccctgcactgtcaactttaccatcttcaaggtcaggatgtatgtggggggcgtggagcacaggctcaacgccgcgtgcaactggactcggggagagcgctgtgacctggaggacagggataggtcagaactcagtccgctgctactgtccacaacagagtggcagatactgccctgttccttcaccaccctaccggctttgtccactggcttgatccacctccatcagaacatcgtggatgtgcaatacttgtatggcatagggtcagccgttgtctccgttgtaatcaaatgggagtacgtcgtgctgcttttccttctcctggcagacgcgcgcgtttgcgcctgcttatggatgatgctgctggtagcccaggctgaggccgccttagagaacctggtggtcctcaatgcagcgtccgtggctggagcgcatggcattctctccttccttgtgttcttctgtgctgcctggtacatcaaaggcaagctggtccctggagcggcatatgctttctatggcgtatggccgctgctcctgctcctgctggcgttaccaccacgagcatacgccatggaccgggagatggccgcatcgtgcggaggcgcggtttttgtaggtctggcactcttgaccttgtcaccacactataaagtgtttctcgctaggctcatatggtggttacaatatttaatcaccagggccgaagcgctcttgcaagtgtggatcccccctctcaacgttcgcgggggccgcgatgccatcatcctcctcacgtgcgcggtccatccagagctaatttttgaaatcaccaaaatcttgctcgccatactcggaccgctcacagtgctccaggcaggcctaactagagtgccgtactttgtacgcgctcaagggctcattcgtgcatgcatgttggtgcggaaagtcgctgggggtcattatgtccaaatggctctcatgaagctaggcgcactgacgggcacgtatctttatgaccatcttactccactgcgggactgggcccacgcgggcctacgagaccttgcgatggcagttgagcccgttatcttttctgacatggagaccaagatcatcacctggggggcagacaccgcggcgtgcggggacatcatctcaggtctacctgtctccgcccgaagggggagggagatactcctaggaccggccgatagttttgaagggcaggggtggcggctccttgcgcctatcacggcctattcccaacaaactaggggcctacttggctgtatcatcactagtctcacaggtcgggacaagaaccgggtcgagggggaggttcaggtggtctccaccgcaacacaatcttttctggcgacctgcgtcaatggcgtgtgttggaccgtctaccacggcgctggcacgaagactttggccggcccgaaaggtccaatcacccaaatgtataccaatgtagaccaagacctcgtcggctggcaggcgccccccggggcgcgttccttgacaccatgcacctgcggcagctcggacctctacttggtcacgaggcatgccgatgtcattccggtgcgccggcggggcgacagcagggggagcctgctctcccccaggcctgtctcctacttgaagggctcctcaggtggtccattgctttgcccctcgggacacgccgtgggcatcttccgggctgctgtgtgcacccggggggtcgcaaaggcggtggactttgtacccgttgagtctatggaaactactatgcggtctccggtctttacggataattcatctcccccggccgtaccgcagacattccaagtggcccatctacacgctcccactggcagcggcaagagcactaaggtgccggctgcatacgcagcccaagggtacaaggtactcgtcctgaacccgtccgttgccgccaccttgggttttggggcgtacatgtctaaggcacatggtgtcgaccctaacatcagaactggggtaaggaccatcactacgggcgctcccattacgtactccacctacggcaagttccttgccgacggtggttgctctgggggtgcctacgacatcataatatgtgatgagtgccactcaactgactcgacttctattttgggcattggcacggtcctggaccaagcggagacggctggagcgcgactcgtcgtgctcgccaccgctacacctccgggatcggtcactgtgccacatcccaacatcgaggaggtggccctgtccaacactggagagattcccttctatggcaaagccatccccatcgagaccatcaagggggggaggcatctcatcttctgccattccaagaagaaatgtgacgagctcgccgcaaagttgtcgggcctcggaatcaacgccgtagcgtactaccggggtcttgatgtgtccgttataccaacaagcggagacgtcgttgtcgtggcaacagacgctctaatgacgggctataccggcgactttgactcggtgatcgactgtaatacatgcgtcacccagacagtcgattttagcttggaccctaccttcaccattgagacgacgaccgtgcctcaagacgcggtgtcgcgctcgcagcggcgaggcaggactggtaggggcagagggggcatatacagatttgtgactccaggggaacggccctcgggcatgttcgattcttcggtcctgtgtgagtgctatgacgcgggctgtgcttggtatgagctcacgcccgccgagacctcagttaggttgcgggcttacctgaatacaccagggttacccgtctgccaggaccatctggagttctgggagagcgtcttcacaggcctcacccacatcgatgcccatttcttgtcccagactaaacaggcaggagacaacttcccctacctggtggcataccaggctacggtgtgcgccagggcccaggctccacctccatcgtgggatcaaatgtggaagtgtctcatacggcttaagcctacactacacgggccaacacccctgttgtacaggctaggggccgtacaaaatgaggtcaccctcacacaccccataaccaaatacatcatggcatgcatgtcggctgacctagaggtcgtcaccagcacctgggtgctggtaggcggggtccttgcggctctggccgcatactgcctgacaacgggcagtgtggtcattgtgggcaggatcatcttgtcagggaagccggctatcattcccgacagggaagttctctaccgagagttcgacgaaatggaggagtgcgcctcacacctcccttacatcgaacagggaatgcagctcgccgagcaattcaagcagaaggcgctcgggttgctgcagacagccaccaagcaagcggaggctgctgctcccgtggtggagtccaagtggcggacccttgaggctttctgggcgaagcacatgtggaacttcatcagcgggatacagtacttagcaggcttgtccactctgcctgggaaccccgcgatagcatccctgatggcattcacagcctctatcaccagcccgctcaccacccaacataccctcttgtttaacatcttggggggatgggtggccgcccaactcgccccccccagcgctgcctcagctttcgtgggcgccggtatcgctggcgcggctgttggcagcataggtcttgggaaggtgcttgtggacatcctggcgggttatggagcaggggtggcaggcgcactcgtggcctttaaggtcatgagtggcgacatgccctccaccgaggatctggtcaacttgctccctgccatcctctctcccggtgccctggtcgtcggagtagtgtgcgcagcaatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcacccagatcctctccagccttaccatcactcagctgttgaagaggctccaccagtggatcaatgaggactgctccacgccatgctccggctcgtggctcagggatgtatgggactggatatgcacggtgttgagtgacttcaagacctggctccagtccaaactcctgccgcggttaccaggagtccctttcctttcatgtcaacgtggatacaagggagtctggcggggagatggcgtcatgcaaaccacctgcccatgtggagcacaaatcgctggacatgtcaaaaacggttccatgaggatcgttgggcctaaaacctgcagcaacacgtggcacggaacattccccatcaacgcgtacaccacaggcccctgcacaccctccccggcgcccaactattctagggcgctgtggcgggtggctgctgaggagtacgtggagatcacgcgggtgggggatttccactacgtgacgggcatgaccgctgacaacgtgaaatgcccatgccaggttccggcccccgaattcttcacagagttggacggggtacggctgcacaggtacgctccggcgtgcaaacctctcctacgggatgaggtcacattccaggtcgggctcaaccaatacccggttgggtcacagctcccatgtgagcccgaaccggatgtaacagtggtcacctccatgctcaccgacccctcccacattacagcagaaacggccaagcgtaggctggccagggggtcgcccccttctttggccagctcttcagccagccagttgtctgcgccttctttgaaggcgacttgcactacccatcatgactccccagatgccgaactcatcgaggccaacctcctgtggcggcaggagatgggcgggaacatcacccgcgtggagtcagagaataaggtagtaatcctagactcttttgacccgctccgagcggaggaggatgagagggaagtatccgttgcggcggagatcctgcgaaagaccaggaaattcccccctgcgatgcccatatgggcacgaccggattacaacccaccactgatagagtcttggagggacccggactacgtccctccggtggtacacgggtgcccattgccacctgccaagacccctccaataccacctccacggagaaagaggacggttgtcctgacagaatccaccgtgtcttctgccttggcggagcttgctacaaagtccttcggcagttccggatcgtcggccgtcgacagcggcacggcaactgcccctcctgatcagcccctcgacagcggagacacaggatccgacgttgagtcgtactcctccatgcccccccttgagggggaaccgggggaccccgatctcagcgacgggtcttggtctactgtgagcgaggaggctggcgaggacgtcgtctgctgctcgatgtcctacacatggacaggcgccttgatcacgccatgcgccgcggaggagagcaagctgcccatcaatgcgttgagcaactctttgctgcgacaccacaacatggtctatgccacaacatcccgcagcgcaagccaacggcagaaaaaggtcacctttgacagactgcaagtcctggacgaccactaccgggacgtgctcaaggagatgaaggcgaaggcgtccacagttaaggctaagcttctatccgtagaggaagcctgcaagctgacgcccccacattcggccaaatcaaaatttggctatggggcgaaggacgtccggaacctgtccagcaaggccgtcaaccacatccactccgtgtggaaggacttgctggaagacgctgaaacaccaattgacaccaccatcatggcgaagaatgaggtcttctgtgttcaaccagagaaaggaggccgcaagccagctcgtcttatcgtattcccagacttgggagttcgtgtgtgcgagaaaatggccctctacgacgtagtctccactcttcctcaggccgtgatgggctcctcatacggattccagtactctcctgggcagcgggtcgagttcctggtgaatgcctggaaatcaaagaagacccctatgggcttcgcatatgacacccgctgctttgactcaacggtcactgagaacgacatccgcgttgaggagtcaatttaccaatgttgtgacttggcccccgaggccagacaggccataaggtcgctcacagagcggctttatattgggggccccctgactaattcaaaagggcagaactgcggttatcgccggtgccgcgccagcggcgtgctgacgaccagctgcggtaatacccttacatgttacttgaaggcctctgcagcctgtcgagctgcaaagctccaggactgcacgatgctcgtgtgcggagacgaccttgtcgttatctgtgaaagcgcgggaactcaggaggacgcggcgagcctacgagtcttcacggaggctatgactaggtactccgccccccccggggacccgccccgaccggaatacgacctggagttgataacatcatgctcctccaacgtgtcggtcgcgcacgatgcaactggcaaacgggtgtactacctcacccgggaccccaccaccccccttgcgcgggctgcgtgggagacagctagacacactccagtcaactcctggctaggcaacatcatcatgtatgcgcccactttgtgggcaaggatgattctgatgactcacttcttctccatccttctagcccaggagcagcttgaaaaagccctagattgtcagatctacggggccacttactccattgaaccacttgacctacctcaaatcattcaacgactccacggtcttagcgcattttcactccatagttactctccaggtgaaatcaatagggtggcttcatgcctcaggaaacttggggtaccgcccttgcgagtctggagacatcgggccagaagtgtccgcgctaagctactgtcccagggggggagggctgcaatatgtggcaagtacctcttcaactgggccgtacggaccaagctcaaactcactccaattccggctgcgtcccggttggacttgtccggctggttcgtcgccggttacagcgggggagacatatatcacagcctgtctcgcgcccgaccccgctggttcatgtggtgcctactcctactttccgtaggggtaggcatctacctgctccccaaccggtgaacggggagctaaacactccaggccaataggccattcctttttttttttttttttttttttttctttttttttccttttttttttttttttttttttttccttttctttcttctttggtggctccatcttagccctagtcacggctagctgtgaaaggtccgtgagccgcatgactgcagagagtgctgatactggcctctctgcagatcatgt

SEQ ID NO: 14 - protein, 3010 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGVARALAQGVRVLEDGVNYATGNLPGCSFSIFLLALLSCLTIPTSAYEVRNVSGVYHVTNDCSNSSIVYEAADMIMHTPGCVPCVRESNFSRCWVALTPTLAARNSSIPTTTIRRHVDLLVGAAAFCSAMYVGDLCGSVFLVSQLFTFSPRRYETVQDCNCSLYPGHVSGHRMAWDMMMNWSPTAALVVSQLLRIPQAIVDMVAGAHWGVLAGLAYYSMVGNWAKVLIVMLLFAGVDGSTRVSGGQVAYNTRGFASLFTPGASQKIQLINTNGSWHINRTALNCNDSLHTGFLAALFYTHKFNASGCPERMASCRPIDKFAQGWGPITHVEPEISDQRPYCWHYAPRPCGIVPASQVCGPVYCFTPSPVVVGTTDRSGVPTYTWGENETDVLLLNNTRPPHGNWFGCTWMNSTGFTKTCGGPPCNIGGVGNNTLTCPTDCFRKHPEATYTKCGSGPWLTPRCIVDYPYRLWHYPCTVNFTIFKVRMYVGGVEHRLNAACNWTRGERCDLEDRDRSELSPLLLSTTEWQILPCSFTTLPALSTGLIHLHQNIVDVQYLYGIGSAVVSVVIKWEYVVLLFLLLADARVCACLWMMLLVAQAEAALENLVVLNAASVAGAHGILSFLVFFCAAWYIKGKLVPGAAYAFYGVWPLLLLLLALPPRAYAMDREMAASCGGAVFVGLALLTLSPHYKVFLARLIWWLQYLITRAEALLQVWIPPLNVRGGRDAIILLTCAVHPELIFEITKILLAILGPLTVLQAGLTRVPYFVRAQGLIRACMLVRKVAGGHYVQMALMKLGALTGTYLYDHLTPLRDWAHAGLRDLAMAVEPVIFSDMETKIITWGADTAACGDIISGLPVSARRGREILLGPADSFEGQGWRLLAPITAYSQQTRGLLGCIITSLTGRDKNRVEGEVQVVSTATQSFLATCVNGVCWTVYHGAGTKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTSILGIGTVLDQAETAGARLVVLATATPPGSVTVPHPNIEEVALSNTGEIPFYGKAIPIETIKGGRHLIFCHSKKKCDELAAKLSGLGINAVAYYRGLDVSVIPTSGDVVVVATDALMTGYTGDFDSVIDCNTCVTQTVDFSLDPTFTIETTTVPQDAVSRSQRRGRTGRGRGGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKYIMACMSADLEVVTSTWVLVGGVLAALAAYCLTTGSVVIVGRIILSGKPAIIPDREVLYREFDEMEECASHLPYIEQGMQLAEQFKQKALGLLQTATKQAEAAAPVVESKWRTLEAFWAKHMWNFISGIQYLAGLSTLPGNPAIASLMAFTASITSPLTTQHTLLFNILGGWVAAQLAPPSAASAFVGAGIAGAAVGSIGLGKVLVDILAGYGAGVAGALVAFKVMSGDMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLSDFKTWLQSKLLPRLPGVPFLSCQRGYKGVWRGDGVMQTTCPCGAQIAGHVKNGSMRIVGPKTCSNTWHGTFPINAYTTGPCTPSPAPNYSRALWRVAAEEYVEITRVGDFHYVTGMTADNVKCPCQVPAPEFFTELDGVRLHRYAPACKPLLRDEVTFQVGLNQYPVGSQLPCEPEPDVTVVTSMLTDPSHITAETAKRRLARGSPPSLASSSASQLSAPSLKATCTTHHDSPDAELIEANLLWRQEMGGNITRVESENKVVILDSFDPLRAEEDEREVSVAAEILRKTRKFPPAMPIWARPDYNPPLIESWRDPDYVPPVVHGCPLPPAKTPPIPPPRRKRTVVLTESTVSSALAELATKSFGSSGSSAVDSGTATAPPDQPLDSGDTGSDVESYSSMPPLEGEPGDPDLSDGSWSTVSEEAGEDVVCCSMSYTWTGALITPCAAEESKLPINALSNSLLRHHNMVYATTSRSASQRQKKVTFDRLQVLDDHYRDVLKEMKAKASTVKAKLLSVEEACKLTPPHSAKSKFGYGAKDVRNLSSKAVNHIHSVWKDLLEDAETPIDTTIMAKNEVFCVQPEKGGRKPARLIVFPDLGVRVCEKMALYDVVSTLPQAVMGSSYGFQYSPGQRVEFLVNAWKSKKTPMGFAYDTRCFDSTVTENDIRVEESIYQCCDLAPEARQAIRSLTERLYIGGPLTNSKGQNCGYRRCRASGVLTTSCGNTLTCYLKASAACRAAKLQDCTMLVCGDDLVVICESAGTQEDAASLRVFTEAMTRYSAPPGDPPRPEYDLELITSCSSNVSVAHDATGKRVYYLTRDPTTPLARAAWETARHTPVNSWLGNIIMYAPTLWARMILMTHFFSILLAQEQLEKALDCQIYGATYSIEPLDLPQIIQRLHGLSAFSLHSYSPGEINRVASCLRKLGVPPLRVWRHRARSVRAKLLSQGGRAAICGKYLFNWAVRTKLKLTPIPAASRLDLSGWFVAGYSGGDIYHSLSRARPRWFMWCLLLLSVGVGIYLLPNR

SEQ ID NO: 15 - DNA, 20 bp, Artificial Sequence
  Primer
tgctcatggtgcacggtcta

SEQ ID NO: 16 - DNA, 20 bp, Artificial Sequence
  Primer
agagccatagtggtctgcgg

SEQ ID NO: 17 - DNA, 20 bp, Artificial Sequence
  Primer
ctttcgcgacccaacactac

SEQ ID NO: 18 - DNA, 18 bp, Artificial Sequence
  Primer
accttccacgagacgctt

SEQ ID NO: 19 - DNA, 22 bp, Artificial Sequence
  Primer
caggatcatctcgaagatcatg

SEQ ID NO: 20 - DNA, 18 bp, Artificial Sequence
  Primer
ggtgcggcggtgatgatg

SEQ ID NO: 21 - DNA, 21 bp, Artificial Sequence
  Primer
cccagagtcggagttgttgag

SEQ ID NO: 22 - DNA, 21 bp, Artificial Sequence
  Primer
ggggtgcgatatttcttcttg

SEQ ID NO: 23 - DNA, 21 bp, Artificial Sequence
  Primer
gagcctgaccaaattcgtacc

SEQ ID NO: 24 - DNA, 20 bp, Artificial Sequence
  Primer
ttcacttctacaaatggacc

SEQ ID NO: 25 - DNA, 20 bp, Artificial Sequence
  Primer
tagcctccgtagccatagcc

SEQ ID NO: 26 - DNA, 22 bp, Artificial Sequence
  Primer
gactcatgaccacagtccatgc

SEQ ID NO: 27 - DNA, 22 bp, Artificial Sequence
  Primer
gaggagaccacctggtgctcag

(19) United States
(12) Patent Application Publication
(10) Pub. No.: US 2011/0129868 A1
(43) Pub. Date: Jun. 2, 2011

(54) CELL CAPABLE OF REPLICATING NOVEL HCV REPLICON, CELL CAPABLE OF REPLICATING FULL-LENGTH HCV RNA, AND USE OF THOSE CELLS

(75) Inventors: Nobuyuki Kato, Okayama-shi (JP); Masanori Ikeda, Okayama-shi (JP)

(73) Assignee: NATIONAL UNIVERSITY CORPORATION OKAYAMA UNIVERSITY, Okayama-shi, Okayama (JP)

(21) Appl. No.: 13/054,905

(22) PCT Filed: Sep. 1, 2009
(86) PCT No.: PCT/JP2009/065263

§ 371 Date: Jan. 19, 2011

(30) Foreign Application Priority Data
Sep. 2, 2008 (JP) 2008-225323

(51) Int. Cl. C12Q 1/02; C12N 15/87; C12N 5/071; C12N 7/00

(52) U.S. Cl. 435/29; 435/440; 435/366; 435/235.1

(57) ABSTRACT

According to the present invention, an HCV replicon-replicating cell is produced by a production method including a step of introducing RNA containing an HCV replicon sequence and a selectable marker gene sequence into a Li23 cell or a cured cell derived from a Li23 cell. Further, a full-length HCV RNA-replicating cell is produced by a production method including a step of introducing RNA containing a full-length HCV genome sequence and a selectable marker gene sequence into a Li23 cell or a cured cell derived from a Li23 cell. The use of these cells enables the construction of an HCV life cycle reproduction system that is derived from a cell line other than the HuH-7 cell line and that has capabilities equivalent to those of an HCV life cycle reproduction system derived from the HuH-7 cell line.

### TECHNICAL FIELD

The present invention relates to a novel HCV replicon-replicating cell, a novel full-length HCV RNA-replicating cell, and use of these cells. More specifically, the invention relates to a HCV RNA replication system and a HCV particle production system using a novel HCV replicon-replicating cell and a novel full-length HCV RNA-replicating cell.

### BACKGROUND ART

Hepatitis C virus (hereinafter, “HCV”) is an RNA virus of the family Flaviviridae, discovered and identified as a causative virus of non-A non-B hepatitis in 1989. Because HCV is a virus that establishes persistent infection, the hepatitis (hepatitis C) caused by HCV infection develops into chronic hepatitis with high probability. It has been elucidated that the hepatitis leads to cirrhosis over the time span of some 20 years, before finally developing into hepatocellular carcinoma.

The estimated number of HCV-infected patients is about two million in Japan alone, and about two hundred million worldwide. The figure becomes even greater with large numbers of so-called asymptomatic carriers unaware of being infected with HCV. This has become a matter of social concern, as seen in the incidence of HCV infection caused by fibrinogen preparations. Currently, the victims of hepatocellular carcinoma in Japan totals about 35,000 per year, about 80% of which is due to HCV infection. The preceding cirrhosis victimizes about 20,000 people annually. Indeed, HCV is a virus that causes serious infections.

A system that can reproduce the repeated cycle of infection, replication, particle production, and reinfection following HCV propagation (HCV lifecycle) would be highly useful for the development of anti-HCV techniques. After the discovery of HCV, many attempts have been made to develop an artificial propagation system using cultured cells and animals; however, no practical system is available. Further, the only model animal of HCV infection is the chimpanzee, and no alternative animal has been found. Use of chimpanzees for drug screening is not practical in terms of scarcity and economy.

In 1999, an HCV replicon system was introduced as a new experiment system that clears the foregoing problems to some extent (see Non-Patent Document 1). An HCV replicon includes HCV genes (non-structural proteins NS3 to NS5B), excluding the genes coding HCV structural proteins. In this system, the HCV subgenome including the NS3 to NS5B regions and the both ends of the genome, essential for HCV genome replication replicates in the cells. The copy number of the HCV subgenome per cell reaches several thousands. Several other HCV strain-derived subgenomic HCV replicon cells have been established afterwards (see, for example, Non-Patent Document 2). In the development of hepatitis C therapeutic drugs, efficacy assessment using such subgenomic HCV replicon systems is necessary, because it is practically impossible to conduct pharmacological tests using large numbers of model animals (chimpanzees).

The influence of HCV structural proteins cannot be assessed in the foregoing subgenomic HCV replicon systems. To overcome this problem, replication systems of full-length HCV genome have been developed, and, thus far, establishment of cells that can replicate the full-length genomes of three HCV strains (N strain, Con-1 strain, and H77 strain) has been reported (full-length HCV RNA replication system; see Non-Patent Documents 3 to 5). Assay systems that can monitor the replication level of HCV genome with a reporter gene are also developed (Non-Patent Document 6, and Patent Document 1). Further, infectious HCV particle-producing cells using JFH1 strain HCV of genotype 2a (HuH-7 cell-derived cloned cells) are established (Non-Patent Document 7).

There is an ongoing global effort directed to developing a specific antiviral agent for HCV using the foregoing techniques (HCV replicon replicating cells, full-length HCV RNA replicating cells, and HCV particle-producing cells that use JFH1 strain HCV).

### CITATION LIST

### Patent Documents

Patent Document 1: Japanese Unexamined Patent Publication No. 2006-325582 (published on Dec. 7, 2006)

### Non-Patent Documents

Non-Patent Document 1: Lohmann et al., Science 285: 110-113 (1999)
Non-Patent Document 2: Kato et al., Biochem. Biophys. Res. Commun. 306: 756-766 (2003)
Non-Patent Document 3: Blight et al., J. Virol. 77: 3181-3190 (2003)
Non-Patent Document 4: Ikeda et al., J. Virol. 76: 2997-3006 (2002)
Non-Patent Document 5: Pietschmann et al., J. Virol. 76: 4008-4021 (2002)
Non-Patent Document 6: Ikeda et al., Biochem. Biophys. Res. Commun. 329: 1350-1359 (2005)
Non-Patent Document 7: Wakita et al., Nat. Med. 11: 791-796 (2005)

### SUMMARY OF INVENTION

### Technical Problem

A specific human hepatoma cell-derived cloned cell line, called HuH-7, is the only cell line that has been used to reproduce the HCV life cycle. It has been elucidated that only a few cell clones among the HuH-7 cells can permit replication of the HCV genome. To verify the results that have been obtained by using HuH-7-derived cells, it is necessary to develop systems that enable reproduction of the HCV life cycle in various cell lines. However, the levels of replication of the HCV replicon and full-length HCV RNA in non-HuH-7 cells are much lower than those in HuH-7 cells, and are not practical for actual use.

The present invention was made in view of the foregoing problems. An object of the present invention is to construct a non-HuH-7 cell-derived HCV life cycle reproduction system that has capabilities equivalent to those of a HuH-7 cell-derived HCV life cycle reproduction system.

### Solution to Problem

The present inventors found that when using specific HCV replicon RNA that is different from the one used to construct the HuH-7 cell-based technique disclosed in Patent Literature 1, the RNA successfully replicates in a specific type of cell that is different from HuH-7 cells. The present invention has been accomplished based on this finding.

A feature of the method of producing an HCV replicon-replicating cell according to the present invention is that the method comprises introducing RNA containing an HCV replicon sequence and a selectable marker gene sequence into a Li23 cell or a cured cell derived from a Li23 cell, and the HCV replicon sequence contains a base sequence encoding an amino acid sequence as set forth in SEQ ID NO: 2 containing amino acid substitutions at Q1112R, K1609E and S2200R or at Q1112R, P1115L and S2200R.

A feature of the method of preparing a full-length HCV RNA-replicating cell is that the method comprises a step of introducing RNA containing an HCV replicon sequence and a selectable marker gene sequence into a cured cell derived from a Li23 cell. The full-length HCV genome sequence may be a base sequence encoding an amino acid sequence as set forth in SEQ ID NO: 2 containing amino acid substitutions at Q1112R, K1609E and S2200R, or at Q1112R, P1115L and S2200R. The base sequence is preferably a sequence as set forth in SEQ ID NO: 7 or 9. The full-length HCV genome sequence may be a base sequence as set forth in SEQ ID NO: 11 or 13.

In the method of producing full-length HCV RNA according to the present invention, the above RNA preferably further contains a reporter gene sequence, and/or preferably further contains an exogenous internal ribosomal entry site (IRES) sequence.

A feature of the screening method according to the present invention is that the method comprises a step of incubating the cell prepared by any one of the above methods with a candidate agent and a step of measuring the level of an HCV gene product or the level of a reporter gene product.

A feature of the method of producing a cured cell according to the present invention is that the method comprises a step of culturing a cell prepared by the above method in a medium containing a pharmaceutical agent having an antiviral action.

A feature of the method of producing an infectious HCV particle according to the present invention is that the method comprises a step of incubating the cured cell with infectious HCV RNA.

### Advantageous Effects of Invention

According to the present invention, a cell capable of replicating an HCV replicon or a cell capable of replicating full-length HCV genome can be produced, and a cell that permits infection with HCV can also be obtained.

### BRIEF DESCRIPTION OF DRAWINGS

FIG. 1(a) represents the structure of HCV-O strain (genotype 1b) HCV genome.

FIG. 1(b) represents the structure of HCV-O strain-derived HCV replicon RNA (ON/3-5B/QR, KE, SR) that includes two adaptive mutations (Q1112R and K1609E) introduced into the NS3 region, and the adaptive mutation S2200R introduced into the NS5A region.

FIG. 1(c) represents the structure of full-length HCV RNA (ON/C-5B/QR, KE, SR) that includes HCV-O strain-derived C (core) to NS2 inserted between the IRES of EMCV and NS3 in the replicon of FIG. 1(b).

FIG. 1(d) represents the structure of RNA (ORN/C-5B/QR, KE, SR) modified to include RL gene inserted between the HCV IRES and NeoR gene of FIG. 1(c) so as to be produced as a fusion protein.

FIG. 1(e) represents the structure of JFH1 strain (genotype 2a)-derived infectious HCV RNA (JFH1) that originates in a fulminant hepatitis patient, and has the structure of the original HCV genome.

FIG. 2 is a diagram representing the procedure of preparing HCV replicon replicating cells and full-length HCV RNA replicating cells.

FIG. 3(a) is a diagram representing the expression of HCV proteins in HCV replicon replicating cells.

FIG. 3(b) is a diagram representing the effects of anti-HCV agents on the replication of an HCV-O strain replicon in HCV replicon replicating cells.

FIG. 4 is a diagram representing the result of full-length HCV RNA quantification in Li23 cell-derived full-length HCV RNA replicating cells.

FIG. 5 is a diagram representing the effect of HCV protein expression in Li23 cell-derived full-length HCV RNA replicating cells.

FIG. 6 is a diagram representing the results of the detection of double-stranded RNA (dsRNA), a replication intermediate of HCV RNA, in OL8 cells, the positive control 0 cells, and the negative control Li23 cells, using an immunofluorescent technique with anti-dsRNA antibodies.

FIG. 7 is a diagram representing the effect of IFN-α on the replication of full-length HCV RNA in Li23 cell-derived full-length HCV RNA replicating cells.

FIG. 8 is a diagram representing the result of gene analysis for HCV replicated in Li23 cell-derived full-length HCV RNA replicating cells.

FIG. 9 is a diagram representing the procedure of preparing reporter gene-carrying HCV replicon-replicating cells derived from Li23 cells, and reporter gene-carrying full-length HCV RNA-replicating cells, shown with the results obtained from these cells.

FIG. 10(a) is a diagram representing the result of full-length HCV RNA quantification in cloned 0RL8 cells.

FIG. 10(b) is a diagram representing the result of full-length HCV RNA quantification in cloned ORL11 cells.

FIG. 11 is a diagram representing the result of gene analysis for HCV replicated in ORL8 cells.

FIG. 12 is a diagram representing the result of gene analysis for HCV replicated in ORL11 cells.

FIG. 13 is a diagram representing HCV protein expression in ORL8 cells and ORL11 cells.

FIG. 14 is a diagram representing the correlation between luciferase activity and HCV RNA level in ORL8 cells and ORL11 cells.

FIG. 15 is a diagram representing the time-dependent anti-HCV activity of IFN-α, using ORL8 cells and ORL11 cells.

FIG. 16 is a diagram comparing the anti-HCV activities of IFN-α using ORL8, ORL11, and OR6 cells.

FIG. 17 is a diagram comparing the anti-HCV activities of IFN-α using sORL8 (pool) cells and sORL11 (pool) cells.

FIG. 18 is a diagram comparing the anti-HCV activities of IFN-β using ORL8, ORL11, and OR6 cells.

FIG. 19 is a diagram comparing the anti-HCV activities of IFN-γ using ORL8, ORL11, and OR6 cells.

FIG. 20 is a diagram comparing the anti-HCV activities of Cyclosporin A (CsA) using ORL8, ORL11, and OR6 cells.

FIG. 21 is a diagram comparing the anti-HCV activities of fluvastatin (FLV) using ORL8, ORL11, and OR6 cells.

FIG. 22 is a diagram comparing the anti-HCV activities of fluvastatin (FLV) using ORL8, ORL11, and OR6 cells.

FIG. 23 is a diagram comparing the anti-HCV activities of simvastatin (SMV) using ORL8, ORL11, and OR6 cells.

FIG. 24 is a diagram comparing the anti-HCV activities of lovastatin (LOV) using ORL8, ORL11, and OR6 cells.

FIG. 25 is a diagram comparing the anti-HCV activities of pitavastatin (PTV) using ORL8, ORL11, and OR6 cells.

FIG. 26 is a diagram comparing the anti-HCV activities of ribavirin (RBV) using ORL8, ORL11, and OR6 cells.

FIG. 27 is a diagram comparing the anti-HCV activities of mizoribine using ORL8, ORL11, and OR6 cells.

FIG. 28 is a diagram comparing the anti-HCV activities of geldanamycin using ORL8, ORL11, and OR6 cells.

FIG. 29 is a diagram comparing the anti-HCV activities of myriocin using ORL8, ORL11, and OR6 cells.

FIG. 30 is a diagram comparing the anti-HCV activities of acetylsalicylic acid (ASA) using ORL8, ORL11, and OR6 cells.

FIG. 31 is a diagram comparing anti-HCV activities by the combined use of IFN-α and CsA using ORL8, ORL11, and OR6 cells.

FIG. 32 is a diagram comparing anti-HCV activities by the combined use of IFN-α and FLV using ORL8, ORL11, and OR6 cells.

FIG. 33 is a diagram comparing anti-HCV activities by the combined use of IFN-α and FLV using ORL8 cells and OR6 cells.

FIG. 34 is a diagram comparing anti-HCV activities by the combined use of IFN-α and FLV using ORL11 cells and OR6 cells.

FIG. 35 is a diagram comparing anti-HCV activities by the combined use of IFN-α and FLV using ORL8 cells and ORL11 cells.

FIG. 36 is a diagram comparing anti-HCV activities by the combined use of IFN-α and FLV using ORL8, ORL11, and OR6 cells.

FIG. 37 is a diagram comparing anti-HCV activities by the combined use of IFN-α and ribavirin (RBV) using ORL8, ORL11, and OR6 cells.

FIG. 38 is a diagram representing the results of the examination of HCV core protein expression in JFH1 strain HCV RNA-introduced Li23 cells, OL8c cells, and OL11c cells.

FIG. 39 is a diagram representing the results of JFH1 strain HCV infection experiment for Li23 cells and OL8c cells.

FIG. 40 is a diagram representing the results of JFH1 strain HCV infection experiment for the clones of various OL cured cells.

FIG. 41 is a diagram representing the results of the examination of infectious HCV particle production from JFH1 strain HCV-infected OL8c and OL11c cells.

FIG. 42 is a diagram representing the results of the examination of infectious HCV particle production from JFH1 strain HCV-infected OL8c cells.

FIG. 43 is a diagram representing the procedure of preparing cured cells by the IFN-γ treatment of OL8c cells and OL11c cells, and the results obtained from these cured cells.

FIG. 44 is a diagram representing the results of the examination of infectious HCV particle production from JFH1 strain HCV-infected ORL8c cells and ORL11c cells.

FIG. 45 is a diagram representing the results of the detection of double-stranded RNA (dsRNA), a replication intermediate of HCV RNA, for ORL8c cells, the positive control JFH1 strain HCV-infected RSc cells, and the negative control mock-infected ORL8c cells, using an immunofluorescent technique with anti-dsRNA antibodies.

FIG. 46(a) is a diagram representing the results of the ELISA measurement of the secretion level of HCV core protein released into the culture supernatants of JFH1 strain HCV-infected ORL8c cells and RSc cells.

FIG. 46(b) is a diagram representing the results of the quantification of HCV RNA level in JFH1 strain HCV-infected ORL8c cells and RSc cells using real-time LightCycler PCR.

FIG. 47(a) is a diagram representing the results of the qualitative comparison of HCV receptor mRNA expression levels in HuH-7 cells, RSc cells, Li23 cells, ORL8c cells, and ORL11c cells.

FIG. 47(b) is a diagram representing the results of the quantitative comparison of HCV receptor mRNA expression levels in HuH-7 cells, RSc cells, Li23 cells, ORL8c cells, and ORL11c cells.

FIG. 48(a) represents the results of the analysis of the expression levels of 1B-4 strain core protein and NS5A protein by Western blotting, confirming the establishment of a cell line capable of replicating full-length HCV RNA derived from non-HCV-O HCV strains.

FIG. 48(b) represents the results of the analysis of the expression levels of KAH5 strain core protein and NS5A protein by Western blotting, confirming the establishment of a cell line capable of replicating full-length HCV RNA derived from non-HCV-O HCV strains.

FIG. 49 is a diagram representing the correlation between luciferase activity and HCV RNA level in cell lines capable of replicating full-length HCV RNA derived from non-HCV-O HCV strains.

### DESCRIPTION OF EMBODIMENTS

### [1] HCV Replicon-Replicating Cell

The present invention provides an HCV replicon-replicating cell. A feature of the HCV replicon-replicating cell according to the present invention is that the cell is produced by introducing HCV replicon RNA having specific adaptive mutations into a specific cell. As used herein, the term “HCV replicon” is interchangeable with the term “subgenomic HCV replicon”. These terms refer to a structural gene comprising the NS3-to-NS5B region of the HCV genome sequence.

A mutation present in HCV ORF may enhance the intracellular replication efficiency of the HCV genome. A mutation having this effect is known as an “adaptive mutation”. A large number of HCV adaptive mutations are known. However, what adaptive mutations are suitable for what conditions is unknown. The present inventors have already established an HCV life cycle reproduction system derived from the HuH-7 cell line (see Patent Document 1). To construct an HCV life cycle reproduction system derived from a non-HuH-7 cell line, the present inventors tried to introduce HCV replicon RNA into various non-HuH-7 cell lines (for example, human hepatoma cell lines, human immortalized liver cell lines, human cholangiocarcinoma cell lines), but were not able to produce a desired transformant. However, as a result of trial-and-error experiments conducted from a unique viewpoint, the present inventors found that when using an HCV replicon sequence that comprises the NS3-to-NS5 region of the HCV genome and contains specific adaptive mutations (Q1112R and K1609E or Q1112R and P1115L) in the NS3 region and a specific adaptive mutation (S2200R) in the NS5A region, the target RNA can be introduced into a human hepatoma cell line, Li23. The combination of adaptive mutations that can be used in the present invention are a combination of “Q1112R, K1609E and S2200R”, or a combination of “Q1112R, P1115L and S2200R”. When using two of the three mutations in each of the above combinations or using a combination different from the above combinations (for example, “P1115L, K1609E and S2200R”, “Q1112R, E1202G and S2200R”, or “E1202G, K1609E and S2200R”), the present invention could not be accomplished. Furthermore, even when using an HCV replicon sequence having such a specific combination of adaptive mutations, the target RNA could not be introduced into cells other than Li23 cells.

A feature of the HCV replicon-replicating cell according to one embodiment of the present invention is that a human hepatoma cell line, Li23, is used as the parent cell and the HCV replicon-replicating cell is prepared by introducing RNA containing an HCV replicon sequence (containing adaptive mutations at Q1112R, K1609E and S2200R or at Q1112R, P1115L and S2200R) and a selectable marker gene sequence into the parent cell.

The RNA to be introduced into the cell according to the present invention is not particularly limited insofar as the RNA contains a selectable marker gene sequence and an HCV replicon sequence. There is no limitation on the selectable marker gene, but drug resistance genes are preferable because of convenience. The drug resistance gene is not particularly limited, and may be suitably selected from known drug resistance genes that can be used for the selection of transformed cells. Specific examples thereof include neomycin resistance genes (neomycin phosphotransferase genes), puromycin resistance genes, blasticidin resistance genes, hygromycin resistance genes, and the like. The HCV replicon sequence is preferably a base sequence as set forth in SEQ ID NO: 3 or 5, and may further contain mutations. In this case, the mutations are not limited to adaptive mutations.

The order of the sequences in RNA introduced into the cell of the present invention is not particularly limited, insofar as a selectable marker gene product and a protein encoded by the HCV replicon sequence can be expressed. The RNA preferably contains two IRESs, which are an IRES for translation of the selectable marker gene and an IRES for translation of the ORF of HCV, thereby maintaining a high level of the translated protein. Although both of the IRESs may be derived from HCV, at least one of the IRESs is preferably a foreign IRES. The mode of HCV genome replication can be maintained by using a foreign IRES. There is no particular limitation on the foreign IRES, and examples thereof include an IRES derived from encephalomyocarditis virus (EMCV), bovine viral diarrhea virus (BVDV) IRES, poliovirus IRES, and the like. EMCV IRES is preferable because of its high activity and wide use.

One example of the order of the sequences in RNA introduced into the cell of the present invention is, from the 5′ end, the HCV IRES sequence, the selectable marker gene sequence, the foreign IRES sequence, the HCV ORF sequence, and the HCV3′ untranslated sequence. However, this example is not limitative. The HCV IRES is an RNA comprising a 5′-untranslated region and a part of the core on the 5′ side. For example, the region from positions 1 to 377 (wherein the 5′-untranslated region is at positions 1 to 341) of the base sequence of the HCV-O strain as set forth in SEQ ID NO: 1 is used in the Examples below. However, this example is not limitative.

The HCV genome sequence contained in RNA introduced into the cell of the present invention may be any sequence derived from HCV. HCV includes attenuated strains and mutant strains as well as pathogenic strains that cause hepatitis C. Although HCV has many genotypes, a sequence derived from any genotype of HCV may be used. Since about 70% of hepatitis C patients in Japan are infected with HCV genotype 1b, genotype 1b is preferable.

Examples of known HCV genotype 1b strains include the HCV-O strain, N strain, Con-1 strain, JT strain, and the like. The present inventors produced the cell of the present invention by using genomic RNA of the HCV-O strain. However, the production method is not limited thereto. The base sequence and the amino acid sequence of the HCV-O strain are as set forth in SEQ ID NO: 1 and SEQ ID NO: 2.

A feature of the HCV replicon-replicating cell according to another embodiment of the present invention is that a cured cell of a Li23-derived full-length HCV RNA-replicating cell (described later) is used as a parent cell, and the HCV replicon-replicating cell is prepared by introducing RNA containing an HCV replicon sequence (containing adaptive mutations at Q1112R, K1609E and S2200R or at Q1112R, P1115L and S2200R) and a selectable marker gene sequence into the parent cell. As used herein, the cell line “derived from a Li23 cell” is interchangeable with the “Li23-derived” cell line. These terms refer to a Li23-derived HCV replicon-replicating cell, a Li23-derived full-length HCV RNA-replicating cell, a cured cell of a Li23-derived HCV replicon-replicating cell, or a cured cell of a Li23-derived full-length HCV RNA-replicating cell. The parent cell used according to this embodiment is preferably an OLc cell described later (see FIG. 2), and more preferably an OL8c, OL11c, or OL14c cell.

The “cured cell” as used herein refers to a cell obtained by culturing a subgenomic HCV replicon-replicating cell or a full-length HCV genome-replicating cell in the presence of a pharmaceutical agent having an antiviral action. The “cured cell” indicates a cell from which the subgenomic HCV replicon has been completely removed, or a cell from which the full-length HCV genome has been completely removed. The term “completely removed” means that no HCV RNA and/or HCV protein is expressed in the cell. Persons skilled in the art can easily confirm whether the cell contains HCV-derived RNA by using a method such as RT-PCR or Northern blotting, and can easily confirm whether HCV protein is expressed by using a method such as Western blotting. Such a cured cell is indicated as “sOLc”, “OLc”, and “ORLc” in FIG. 2, and referred to as a “cured cell derived from a Li23 cell” in the specification. Particularly, “OLc” and “ORLc” are also referred to as “cured cells of Li23-derived full-length HCV RNA-replicating cells”.

The pharmaceutical agent having an antiviral action is not particularly limited, insofar as a cured cell can be obtained by adding the agent to a medium. However, the pharmaceutical agent is preferably an agent having an anti-HCV action, more preferably IFN, Cyclosporin A (CsA), or the like, and particularly preferably IFN. Examples of IFN include IFN-α, IFN-β, IFN-γ, and the like. One example of the method of treating the cell using IFN comprises culturing the cell in a medium containing IFN-α in a concentration of 500 IU/ml for 2 weeks. However, the concentration and duration of the treatment may be suitably changed, while confirming whether the desired cured cell has been obtained.

The present invention further provides a method of producing an HCV replicon-replicating cell. A feature of the production method according to one embodiment of the present invention is that the method comprises a step of introducing RNA containing an HCV replicon sequence (containing adaptive mutations at Q1112R, K1609E and S2200R or at Q1112R, P1115L and S2200R) and a selectable marker gene sequence into a Li23 cell. A feature of the production method according to another embodiment of the present invention is that the method comprises a step of introducing RNA containing an HCV replicon sequence (containing adaptive mutations at Q1112R, K1609E and S2200R or at Q1112R, P1115L and S2200R) and a selectable marker gene sequence into a cured cell derived from a Li23 cell.

The cells prepared by the present inventors have been deposited in a depository for Okayama University, the National University Corporation Okayama University Intellectual Property Headquarters (1-1, Tsushima-naka 1-chome, Okayama-shi). The deposit numbers are as shown below.

TABLE 1

| Cell Name | Deposit Number |
|---|---|
| Li23 | OP-KITAKU-0001 |
| sOL | OP-KITAKU-0002 |
| OL8 | OP-KITAKU-0003 |
| OL11 | OP-KITAKU-0004 |
| OL14 | OP-KITAKU-0005 |
| sORL8 (pool) | OP-KITAKU-0006 |
| sORL11 (pool) | OP-KITAKU-0007 |
| ORL8 | OP-KITAKU-0008 |
| ORL11 | OP-KITAKU-0009 |
| 1B-4RL8 | OP-KITAKU-0010 |
| KAH5RL8 | OP-KITAKU-0011 |
| 1B-4RN/C-5B | OP-KITAKU-0012 |
| OR6 | OP-KITAKU-0013 |

These cells were also deposited at the National Institute of Advanced Industrial Science and Technology, International Patent Organism Depository (Tsukuba Central 6, 1-1-1 Higashi, Tsukuba, Ibaraki, 305-8566, Japan) on Jul. 31, 2008. A request for a transfer to the international deposit was received on Jul. 30, 2009. The accession numbers are as shown below.

TABLE 2

| Cell Name | Deposit Number |
|---|---|
| Li23 | FERM ABP-11150 |
| sOL | FERM ABP-11151 |
| OL8 | FERM ABP-11152 |
| OL11 | FERM ABP-11153 |
| OL14 | FERM ABP-11154 |
| sORL8 (pool) | FERM ABP-11155 |
| sORL11 (pool) | FERM ABP-11156 |
| ORL8 | FERM ABP-11157 |
| ORL11 | FERM ABP-11158 |
| 1B-4RL8 | FERM ABP-11159 |
| KAH5RL8 | FERM ABP-11160 |
| OR6 | FERM ABP-11161 |
| 1B-4RN/C-5B | FERM ABP-11162 |

### [2] Full-Length HCV RNA-Replicating Cell

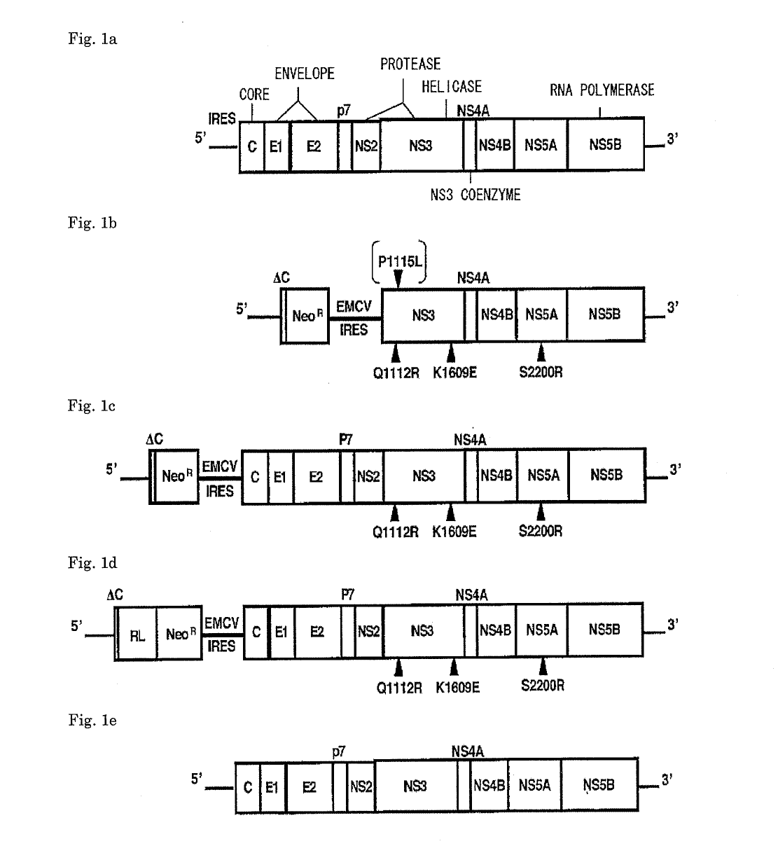

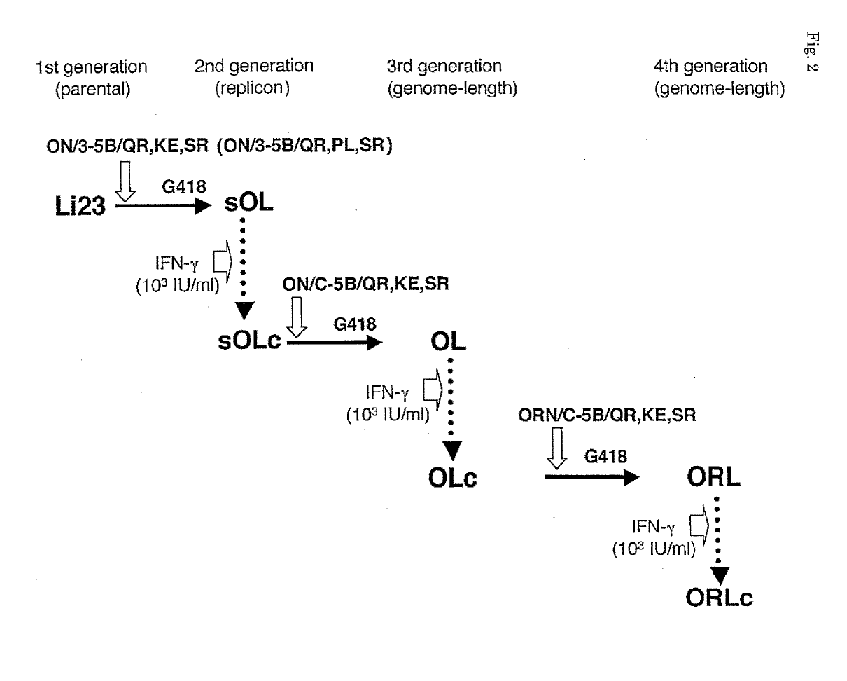

The present invention provides a full-length HCV RNA-replicating cell. A feature of the full-length HCV RNA-replicating cell according to the present invention is that a full-length HCV genome is introduced into a specific cell. The term “full-length HCV genome” as used herein refers to RNA containing all regions (C region to NS5B region) of the HCV genome shown in FIG. 1(a), and is interchangeable with “full-length HCV RNA”.

The RNA introduced into the full-length HCV RNA-replicating cell according to the present invention is not particularly limited insofar as the RNA contains a selectable marker gene sequence and a full-length RNA sequence. Preferably, the RNA further contains a reporter gene because a reporter assay can thereby easily be performed with high sensitivity. There is no particular limitation on the selectable marker gene. However, drug resistance genes are preferable because of convenience. The drug resistance gene is not particularly limited, and can be suitably selected from known drug resistance genes that are usable for the selection of transformed cells. Specific examples thereof include neomycin resistance genes (neomycin phosphotransferase genes), puromycin resistance genes, blasticidin resistance genes, hygromycin resistance genes, and the like. The full-length HCV RNA sequence is preferably a base sequence as set forth in SEQ ID NO: 1, 7, 9, 11, or 13, and may further contain mutations. In this case, the mutations are not limited to adaptive mutations.

The reporter gene that is optionally contained in RNA introduced into the full-length HCV RNA-replicating cell according to the present invention is not particularly limited. Examples thereof include luciferase genes, alkaline phosphatase genes, β-lactamase genes, chloramphenicol acetyltransferase genes, and the like. Luciferase genes are preferable. In general, firefly luciferase genes or Renilla luciferase genes are used as luciferase genes. Any of them may be used in the present invention. Renilla luciferase genes are preferable in view of the short length of the gene.

The RNA introduced into the full-length HCV RNA-replicating cell of the present invention preferably comprises two IRESs, which are an IRES for translation of a selectable marker gene and an IRES for translation of the ORF of HCV, thereby maintaining a high level of the translated protein. Although both of the IRESs may be derived from HCV, at least one of them is preferably a foreign IRES. The mode of replication of the HCV genome can be maintained by using a foreign IRES. The foreign IRES is not particularly limited, and examples thereof include an IRES from encephalomyocarditis virus (EMCV), bovine viral diarrhea virus (BVDV) IRES, poliovirus IRES, and the like. EMCV IRES is preferable because of its high activity and wide use.

One example of the order of the sequences in RNA introduced into the full-length HCV RNA-replicating cell is, from the 5′ end, the HCV IRES sequence, (optionally) the reporter gene sequence, the selectable marker gene sequence, the foreign IRES sequence, the HCV ORF sequence, and the HCV3′ untranslated sequence. However, this example is not limitative.

The full-length HCV RNA-replicating cell according to one embodiment of the present invention comprises an sOLc cell into which a full-length HCV genome has been introduced. The sOLc cell is obtained by introducing RNA containing an HCV replicon sequence (containing adaptive mutations at Q1112R, K1609E and S2200R) and a selectable marker gene sequence into a Li23 cell to produce an HCV replicon-replicating cell (sOL cell) and culturing the HCV replicon-replicating cell in the presence of a pharmaceutical agent having an antiviral action (see FIG. 2). The full-length HCV RNA-replicating cell according to this embodiment is an OL cell as set forth in FIGS. 2. OL1 to OL14 cells are preferable, and OL8, OL11, and OL14 cells are more preferable.

The full-length HCV RNA-replicating cell according to another embodiment of the present invention comprises an OLc cell into which a full-length HCV genome has been introduced. The OLc cell is obtained by culturing an OL cell in the presence of a pharmaceutical agent having an antiviral action (see FIG. 2). The full-length HCV RNA-replicating cell according to this embodiment comprises RNA containing a full-length HCV genomic sequence and a selectable marker gene sequence, and is preferably an ORL cell as set forth in FIG. 2 (containing adaptive mutations at Q1112R, K1609E and S2200R). ORL8-1 to ORL8-9 cells and ORL11-1 to ORL11-16 cells are more preferable, and an ORL8-9 cell (hereinafter referred to as an “ORL8 cell”) or an ORL11-5 cell (hereinafter referred to as an “ORL11 cell”) are particularly preferable.

The full-length HCV RNA-replicating cell according to another embodiment of the present invention comprises an ORLc cell into which a full-length HCV genome has been introduced. The ORLc cell is obtained by culturing an ORL cell in the presence of a pharmaceutical agent having an antiviral action (see FIG. 2). The full-length HCV RNA-replicating cell according to this embodiment is produced by introducing RNA containing a full-length HCV genomic sequence and a selectable marker gene sequence. The full-length HCV RNA-replicating cell according to this embodiment permits replication of RNA of the infectious HCV JFH1 strain as well as a novel HCV strain (particularly replication of a long RNA having, for example, a luciferase gene (12 kb)). More specifically, the full-length HCV genomic sequence used in this embodiment may be derived from any HCV strain. The HCV strain to be used is preferably an HCV-O, 1B-4, or KAH-5 strain. The full-length HCV RNA-replicating cell according to this embodiment is preferably a 1B-4RL8 or KAH5RL8 cell. The base sequence (SEQ ID NO: 11) and the amino acid sequence (SEQ ID NO: 12) of the HCV 1B-4 strain, and the base sequence (SEQ ID NO: 13) and the amino acid sequence (SEQ ID NO: 14) of the KAH5 strain have been registered in GenBank under the accession numbers AB442219 and AB442220, respectively.

The full-length HCV RNA-replicating cell according to the present invention can replicate a full-length HCV genome, and express a reporter gene product when necessary. The full-length HCV RNA-replicating cell according to the present invention is a cell into which RNA containing a selectable marker gene sequence and a full-length HCV genomic sequence (and optionally a reporter gene sequence) has been introduced, and may be any cell that can replicate a full-length HCV genome, preferably a cell that can express a reporter gene product. When the RNA contains a reporter gene sequence, the expression level of the reporter gene product and the amount of HCV RNA replication in the full-length HCV RNA-replicating cell according to the present invention are very closely correlated with each other. Accordingly, quantification of the reporter gene product enables easy monitoring of the replication level of the full-length HCV genome. Furthermore, the full-length HCV RNA-replicating cell according to the present invention is very useful for functional analysis of HCV, including the effects of structural proteins, which is not possible with a subgenomic HCV replicon. More specifically, the full-length HCV RNA-replicating cell according to the present invention enables easy and quick functional analysis of HCV, screening of substances having anti-HCV action, etc.

The present invention provides a method of producing a full-length HCV RNA-replicating cell. A feature of the production method according to one embodiment of the present invention is that the method comprises a step of introducing into an sOLc cell RNA containing a full-length HCV genomic sequence and a selectable marker gene sequence. The RNA may further contain a reporter gene sequence. A feature of the production method according to another embodiment of the present invention is that the method comprises introducing into an OLc cell RNA containing a full-length HCV genomic sequence and a selectable marker gene sequence. The RNA may further contain a reporter gene sequence. A feature of the production method according to another embodiment of the present invention is that the method comprises introducing into an ORLc cell RNA containing a full-length HCV genomic sequence and a selectable marker gene sequence. The RNA may further contain a reporter gene sequence.

### [3] Screening Method

The present invention provides a method of screening a substance having an anti-HCV action. The screening method according to the present invention is not particularly limited, insofar as the method includes a step of incubating the cell of the present invention with a candidate agent. When the RNA introduced into the above cell contains a reporter gene sequence, the method may further include a step of measuring the level of a reporter gene product. When RNA introduced into the cell does not contain a reporter gene sequence, the method may further include a step of measuring the level of an HCV gene product. The measured levels may be compared with preset reference values. More preferably, the reporter gene product level or the HCV gene product level achieved by not incubating the cell with a candidate agent is measured, and is compared with the corresponding level achieved by incubating the cell with a candidate agent. This screening method enables easy and quick screening of a large number of test substances.

In the screening method according to the present invention, the anti-HCV action is an inhibitory effect on the replication of a full-length HCV genome. Thus, the screening method according to the present invention is a method of screening a substance having an inhibitory action on the replication of a full-length HCV genome.

The screening method according to the present invention can screen a substance that suppresses the replication of viruses closely related to HCV, and viruses whose mode of replication is similar to that of HCV. Examples of viruses closely related to HCV include viruses belonging to the Pestivirus genus and the Flavivirus genus of the Flaviviridae family. Examples of viruses belonging to the Flavivirus genus include Japanese encephalitis virus, yellow fever virus, West Nile virus, and the like. Examples of viruses belonging to the Pestivirus genus include hog colera virus, bovine viral diarrhea virus, and the like.

Further, the screening method according to the present invention enables the screening of a substance that suppresses the replication of a virus whose mode of replication is similar to that of HCV. A feature of HCV replication is that the replication occurs entirely within the cytoplasm, and no viral genome is present in the nucleus. Accordingly, the screening method according to the present invention can screen a substance that suppresses the replication of a virus whose mode of replication is as described above.

The test substance and the cell can be brought into contact with each other by dissolving or suspending the test substance in a medium. Accordingly, the test substance may be any material that can be dissolved or suspended in a medium.

The method of measuring the level of a reporter gene product can be selected from known methods according to the reporter gene used. For example, when a luciferase gene is used as a reporter gene, a cell lysate prepared by dissolving the cell in a buffer containing a surfactant or the like is used as a test sample, and the amount of emission may be measured by an apparatus, such as a luminometer. In this measurement, commercially available luciferase assay reagents and luciferase assay kits can be suitably used.

Whether the test substance has an anti-HCV action or not can be determined by comparing the thus obtained level of the reporter gene product with the level of a reporter gene product in a cell not brought into contact with the test substance. Further, if the level of the reporter gene product in the cell brought into contact with a test substance in a concentration that does not reduce the cell growth potential is lower than that in the cell not brought into contact with the test substance, the test substance is evaluated as having an anti-HCV action. Preferably, when the level is not more than 50%, and more preferably 10% or less, the test substance is evaluated as having an anti-HCV action.

A more preferable criterion is the level obtained by applying IFN-α, to the screening method according to the present invention, or the level obtained by applying IFN-α and ribavirin to the screening method according to the present invention, both being standard methods for chronic hepatitis C treatment. A highly useful therapeutic agent for hepatitis C can be found if the HCV replication level achieved by the therapeutic agent is lower than the levels achieved by pharmaceuticals used in the current standard therapeutic method for hepatitis C.

The screening method according to the present invention enables at least selection of candidates for the active ingredient of a hepatitis C therapeutic agent. Since the only animal model for HCV infection is the chimpanzee, it is currently impossible to perform a pharmacological test using a large number of animals. It is thus expected that the screening method according to the present invention will be a vital method for the evaluation of drug efficacy in the development of a therapeutic agents for hepatitis C.

### [4] Screening Kit

The present invention provides a screening kit for screening a substance having an anti-HCV action. The screening kit according to the present invention is not particularly limited, insofar as the kit contains the cell of the present invention. When RNA introduced into the cell contains a reporter gene sequence, the kit may further contain a reagent for measuring the level of a reporter gene product. When RNA introduced into the cell does not contain a reporter gene sequence, the kit may further contain a reagent for measuring the level of an HCV gene product. This screening kit enables simple and effective implementation of the screening method according to the present invention.

The term “kit” as used herein refers to a package (e.g., a bottle, a plate, a tube, a dish, or the like) containing a specified material, and includes instructions for use of the specified material. The instructions may be written or printed on paper or other media, or committed to electronic media such as magnetic tape, computer-readable disks or tape, CD-ROM, and the like.

The screening kit according to the present invention may further contain items other than the cell of the present invention. Such components of the kit other than the cell are not particularly limited; necessary reagents, apparatuses, etc., may be selectively incorporated as components of the kit.

A person skilled in the art who reads this specification will easily understand that the screening kit according to the present invention can be used in the same manner as the screening method of the present invention described above.

### [5] Method of producing an infectious HCV particle

The present invention provides a method of producing an infectious HCV particle. The method of producing an infectious HCV particle according to the present invention may be any method comprising a step of introducing infectious HCV RNA into a cell (a “cured cell derived from a Li23 cell”) obtained by culturing the cell of the present invention in a medium containing a pharmaceutical agent having an antiviral action, or a step of incubating the cell with infectious HCV. Examples of cells preferably used in the method of the present invention include, but are not limited to, ORL8c and ORL11c cells.

### EXAMPLES

### 1: Reagents and Procedures

### Reagents Used

Fluvastatin was purchased from Calbiochem and LKT Laboratories. IFN-α, IFN-β, and IFN-γ were purchased from Sigma. Cyclosporin A (CsA), myriocin, and acetylsalicylic acid were purchased from Sigma. Pravastatin, simvastatin, lovastatin, and geldanamycin were purchased from Wako chemical. Pitavastatin was purchased from Tronto Research. Ribavirin was purchased from Yamasa. Mizoribine was provided by Asahi Kasei.

Li23 cells

The human hepatoma cell line Li23 was cultured using 500 ml of F-12 medium (Invitrogen 11765-054) and 500 ml of D-MEM medium (Sigma D5796) (a total volume of 1 L), with addition of the following materials.

| | Final Concentration | |
|---|---|---|
| Materials Added (Group A) | | |
| EGF (Toyobo EGF-201) | 50 | ng/ml |
| Insulin (Sigma I-6634) | 10 | μg/ml |
| Hydrocortison (Sigma H-0888) | 0.36 | μg/ml |
| Transferrin (Sigma T-2252) | 5 | μg/ml |
| Linoleic acid (Sigma L-1012) | 5 | μg/ml |
| Selenium (Sigma S-9133) | 20 | ng/ml |
| Prolactin (Sigma L6520) | 10 | ng/ml |
| FBS (Biological Industries 04-001-1A) | 1% (v/v) | |
| Material Added (Group B) | | |
| Gentamycin (Invitrogen 15750-060) | 10 | μg/ml |
| Kanamycun-monosulfate (Sigma K-4000) | 0.2 | mg/ml |
| Fungizone (IBL 33605) | 0.5 | μg/ml |

FBS was used after 30-min incubation at 56° C. The cell line Li23 shows EGF-dependent proliferation, and does not easily proliferate under the HuH-7 cell medium conditions (10% Fetal Bovine Serum (FBS)) commonly used for the HCV replication model. The Li23 cell line containing the HCV subgenomic replicon or full-length HCV genome was maintained in a medium that contained G418 at a concentration of 0.3 mg/ml (Invitrogen). The same culture was used without G418 for the cured cells (described later).

It was confirmed that the Li23 cells had the characteristics of the liver cells as do the HuH-7 cells, as follows. Li23 cells were examined with regard to expression of genes specific to the liver cells, or expression of 11 genes reported to have high expression levels in the liver cells (Aly H H et al., J. Hepatology, 46: 26-36, 2007), using a standard RT-PCR method. The results were compared with those from the human hepatoma cell line HuH-7, human cervical cancer cells (HeLa cells), or human embryonic kidneys (HEK293 cells). It was found that the expression level of each gene in the Li23 cells was about the same as that in HuH-7 cells, whereas the genes were not detected in HeLa and HEK293 cells (the results are not presented).

### Northern Blot Analysis

Total RNA was extracted from the subject cells using an RNeasy Mini Kit (Qiagen), according to the manufacturer's experiment protocol. The extracted RNA was quantified by absorbance measurement at the wavelength of 260 nm. HCV RNA and β-actin RNA were detected with 4 μg of the RNA. Specifically, specific RNA detection was made using a Northern Max Kit (Ambion), according to the manufacturer's experiment protocol. The RNA sample was subjected to electrophorisis, and the gel was blotted on a Hybond-N+nylon membrane (Amersham-Pharmacia Biotech). The RNA was fixed to the membrane using a UV Crosslinker (Stratagene), and the 28S rRNA portion on the membrane was stained with ethidium bromide. The membrane was cut about 1 cm below the 28S rRNA band. HCV RNA was contained in the upper part of the removed membrane. β-actin mRNA was contained in the lower part. For the specific detection of HCV RNA, a minus-strand riboprobe complementary to the digoxigenine-labeled HCV NS5B region was synthesized and used according to the manufacturer's experiment protocol attached to a digoxigenine labeling kit (Roche). Alkali phosphatase-labeled anti-digoxigenin antibodies were used for the detection of the riboprobe that had specifically bound to the HCV RNA. After reaction using a CSPD (Roche), the patterns were exposed on an X-ray film for specific detection of HCV RNA. β-actin RNA was detected in the same manner.

### Western Blot Analysis

An SDS-containing sample buffer (100 μl) was added to the cells cultured in a 6-well culture plate, and the cell lysate was collected. After 10-min sonication using an ultrasonic homogenizer, each sample was supplemented with 10 μl of 2-mercaptoethanol, and treated at 100° C. for 3 min. 10 to 20 μl of the sample was subjected to 10% SDS-PAGE, and the proteins were transferred to a membrane (PVDF membrane). The protein-transferred membrane was blocked for 60 min with 0.1% Tris buffer that contained 5% skim milk (10 mM Tris (pH 7.5), 150 mM NaCl, 0.1% Tween20). Then, the membrane was contacted with a solution of antibodies against the HCV proteins and the β-actin protein diluted 1,000 times with 0.1% Tris buffer, and a reaction was allowed for 60 min. After washing the membrane three times with 0.1% Tris buffer 5 min each time, the membrane was contacted with a 0.1% Tris buffer supplemented with HRP-labeled mouse secondary antibodies diluted 1,000 times, and a reaction was allowed for 60 min. The membrane was washed three times with 0.1% Tris buffer, 20 min each time. The proteins were allowed to chemiluminesce with a Renaissance™ Luminol Western Blot Chemiluminescence Reagent Plus (NEN Life Science), and exposed on an X-ray film (KODAK BioMax).

The antibodies used in the experiments were anti-core antibodies (Institute of Immunology), anti-E1 antibodies (a gift from Dr. Kohara, Tokyo Metropolitan Institute of Medical Science), anti-E2 antibodies (see the reference: Microbiol. Immunolo. 42, 875-877, 1998), anti-NS3 antibodies (Novocastera Laboratories), anti-NS4A antibodies (a gift from Dr. Takamizawa, Osaka University), anti-NS5A antibodies (a gift from Dr. Takamizawa, Osaka University), anti-NS5B antibodies (a gift from Dr. Kohara, Tokyo Metropolitan Institute of Medical Science), and anti-β-actin antibodies (Sigma).

### Plasmid Construction

Plasmid pON/C-5B includes a neomycin phosphotransferase (Neo)-encoding sequence downstream of the HCV IRES (internal ribosomal entry site), and a full-length HCV-O protein-encoding sequence downstream of the Encephalomyocarditis virus (EMCV) IRES.

First, a plasmid pHCV-O that contained a genotype 1b HCV-O full-length cDNA was constructed from HCV positive serum, using two fragments. The two fragments are EcoRI-MluI fragment (corresponding to position 45-2528 of the HCV genome) derived from the pBR322/16-6 described in the reference (Kato et al. J. Gen. Virol. 79: 1859-1869 (1998)), and MluI-SpeI fragment (corresponding to position 2528-3420 of the HCV genome) derived from the PCR product of serum 1B-2. These fragments were ligated to the EcoRI-SpeI site of pNSS1RZ2RU having a 1B-2R1 sequence (see Non-Patent Document 2) to construct pHCV-O.

To obtain a fragment for constructing pON/C-5B, the EMCV IRES was fused with the coding sequence of core protein using overlapping PCR. The resulting DNA was digested with RsrII and ClaI, and ligated to the ClaI-XbaI site of pHCV-O with a XbaI-RsrII fragment of pNSS1RZ2RU.

Plasmid pON/3-5B was constructed in a manner described in Non-Patent Document 2 in detail. Specifically, as described in Non-Patent Document 2, the region of the RNA extracted from sO cells corresponding to position 3474-9185 of HCV-O gene was amplified by RT-PCR method, and the amplified RNA was ligated to the SpeI-BsiwI site of pNSS1RZ2RU to construct pON/3-5B.

Further, according to the method of Ikeda et al. (see Non-Patent Document 4), Q1112R, K1609E, and S2200R mutations, or Q1112R, P1115L, and S2200R mutations were introduced into the pON/3-5B using QuickChange mutagenesis (Stratagene) to construct pON/3-5B/QR, KE, SR, or pON/3-5B/QR, PL, SR. Further, Q1112R, K1609E, and 52200R mutations, or Q1112R, P1115L, and S2200R mutations were introduced into the pON/C-5B to construct pON/C-5B/QR, KE, SR, or pON/C-5B/QR, PL, SR. The plasmid pORN/C-5B/QR, KE, SR was constructed by introducing the PCR product of Renilla luciferase gene (Promega) to the AscI site upstream of the Neo gene in pON/C-5B/QR, KE, SR. Each plasmid contains a T7 promoter sequence on the 5′ side of the inserted gene.

### RNA Synthesis

The plasmid DNA was linearized by cutting with XbaI, and RNA synthesis was performed using a T7 MEGAscript Kit (Ambion) according to the manufacturer's experiment protocol. After being precipitated with lithium chloride, the RNA was washed with 75% ethanol, and dissolved in RNase-free water.

### Quantification of HCV RNA

Total RNA was extracted from the HCV RNA replicating cells using an RNeasy Mini Kit (Qiagen) according to the manufacturer's experiment protocol. First, using 2 μg of RNA as a template, reverse transcription (RT) reaction was performed with SuperScript™ II reverse transcriptase (Invitrogen) and the primer 319R below according to the manufacturer's experiment protocol. HCV RNA was quantified by real-time LightCycler PCR, using the resulting cDNA as a template. Real-Time LightCycler PCR was performed based on the method previously reported by the present inventors (reference: Acta Med. Okayama 56, 107-110, 2002), using the primers 104 and 197R below.

| (SEQ ID NO: 15) | |
|---|---|
| 319R: | 5′-TGCTCATGGTGCACGGTCTA-3′ |
| (SEQ ID NO: 16) | |
| 104: | 5′-AGAGCCATAGTGGTCTGCGG-3′ |
| (SEQ ID NO: 17) | |
| 197R: | 5′-CTTTCGCGACCCAACACTAC-3′. |

### Luciferase Reporter Assay

Renilla luciferase was quantified by collecting cells using a Renilla Luciferase Assay System (Promega) according to the manufacturer's experiment protocol.

### 2: HCV Genome

FIG. 1 is a schematic illustration showing the structure of HCV genome, the structure of RNA introduced to subgenomic HCV replicon cells, the structure of RNA introduced to full-length HCV genome replicating cells, and the structure of RNA introduced to full-length HCV genome replicating cells expressing a luciferase gene product.

FIG. 1(a) shows an HCV-O strain (genotype 1b). The HCV genome is a positive-chain, single-stranded RNA of about 9.6 kb (SEQ ID NO: 1), 90% of which is a single large ORF producing a polyprotein of about 3,000 amino acids (SEQ ID NO: 2). The polyprotein is processed by host protease in the first half that ends with p7, whereas the remaining portion is processed by two proteases encoded by NS2 and NS3. In the end, at least 10 virus proteins are produced. The region ending with E2 is called a structural region that forms viral particles, and NS2 to NS5B are called a non-structural region. The regions necessary for HCV RNA replication are known to include the 5′ untranslated region (HCV IRES) including a region that encodes the first 12 amino acids of the core, a region from NS3 to NS5B, and a 3′ end region. The HCV-O strain is an HCV strain that belongs to genotype 1b, which accounts for about 70% of patients in Japan, and was isolated from healthy carriers (HCV-infected individuals with normal liver function).

The RNAs used in the present invention are shown in FIG. 1(b) to FIG. 1(e). FIG. 1(b) shows HCV-O strain-derived HCV replicon RNA (ON/3-5B/QR, KE, SR) that includes two adaptive mutations (Q1112R and K1609E) introduced into the NS3 region, and adaptive mutation S2200R introduced into the NS5A region. The HCV-O strain-derived HCV replicon RNA (ON/3-5B/QR, PL, SR) to which P1115L adaptive mutation is introduced in place of K1609E also has this structure. The full-length HCV RNA (ON/C-5B/QR, KE, SR) shown in FIG. 1(c) includes HCV-O strain-derived C (core) to NS2 inserted between the internal ribosomal entry site (IRES) of Encephalomyocarditis virus (EMCV) and NS3 in the replicon (ON/3-5B/QR, KE, SR (FIG. 1(b)), and thus has a structure 1.4 kb longer than the original HCV genome (a full-length of 11 kb). The RNA (ORN/C-5B/QR, KE, SR) shown in FIG. 1(d) is modified to include Renilla luciferase gene (RL gene) between the HCV IRES and NeoR gene of the full-length HCV RNA (ON/C-5B/QR, KE, SR (FIG. 1(c)) so as to be produced as a fusion protein, and has a structure 2.4 kb longer than the original HCV genome (a full-length of 12 kb). The RL gene allows for the quantification of HCV RNA replication levels through RL activity measurement (referred to as “reporter assay”). The HCV RNA (JFH1 strain) shown in FIG. 1(e) has the structure of the original HCV genome, specifically, infectious HCV RNA derived from the JFH1 strain (genotype 2a) that originates in a fulminant hepatitis patient.

These five kinds of RNA were obtained by in vitro synthesis using a T7 MEGAscript (Ambion) after the plasmids (pON/3-5B/QR, KE, SR; pON/3-5B/QR, PL, SR; pON/C-5B/QR, KE, SR; pORN/C-5B/QR, KE, SR; pJFH1) containing these RNA sequences were linearized by cutting with restriction enzyme XbaI. The plasmid containing the full-length JFH1 cDNA was provided by Tokyo Metropolitan Organization for Medical Research based on research material transfer agreement.

### 3: HCV Replicon Replicating Cell Line

### 3-1 Preparation of Cell Line

The RNA (10 μg) synthesized in vitro from pON/3-5B/QR, KE, SR or pON/3-5B/QR, PL, SR was introduced into Li23 cells (8×10⁶) according to the method described in Non-Patent Document 2. After 2 days, the medium was replaced with medium containing G418 (0.3 mg/ml) and NaHCO₃ (0.15%). The cells were cultured for 3 weeks with medium replacement every 4 days, and cells that showed sustained high levels of replicon RNA replication were obtained as G418-resistant colonies. Some of the colonies (one plate) were stained with Coomassie Brilliant Blue (CBB). Similar replicon RNA introduction experiments were conducted using other cell lines, including other human hepatoma cell lines (HuH-6, Li21, Li24), human immortalized liver cell lines (PH5CH, OUMS29, IHH10.3, IHH12), and human bile duct cancer cell line (HuH28). However, no G418-resistant cell colonies were obtained (the results are not presented). The Li23 cells to which the RNA was not introduced were completely killed by the G418 contained in the medium (the results are not presented).

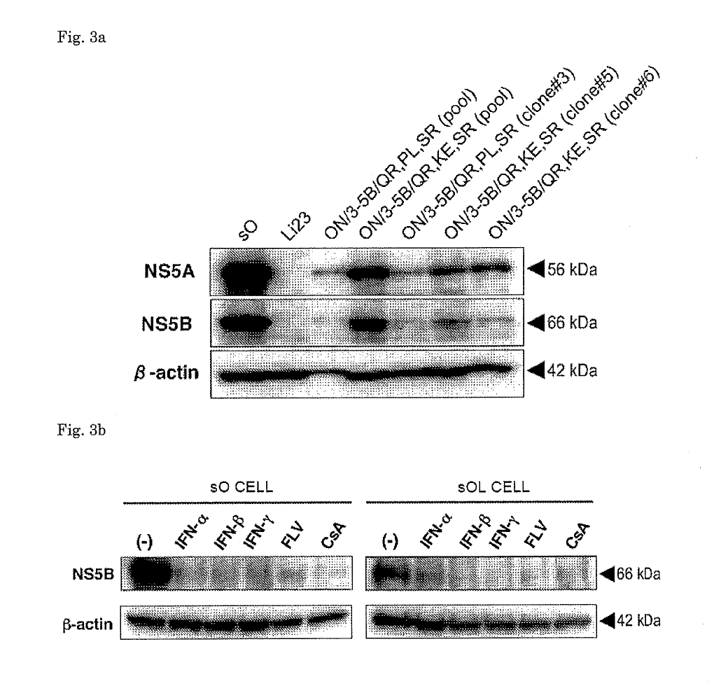

The expression levels of the HCV proteins in the G418-resistant cells were analyzed by Western blotting. The NS5A and NS5B proteins were detected by an ordinary method using NS5A and NS5B antibodies, respectively. The sO cells (HuH-7 cell-derived cells that show efficient replication of HCV-O strain-derived replicon RNA) reported in Non-Patent Document 2 by the present inventors were used for comparison. β-actin detection using β-actin antibody was performed in parallel to find the amounts of proteins used in the analysis. FIG. 3(a) shows that the expression levels of NS5A and NS5B proteins are far greater in cells obtained by introducing ON/3-5B/QR, KE, SR RNA than in cells obtained by introducing ON/3-5B/QR, PL, SR RNA. In the case of ON/3-5B/QR, KE, SR, it was also found that colony-pooled cells had higher expression levels than cloned cells. The pooled cells (ON/3-5B/QR, KE, SR (pool)) were used as sOL cells in the next step without selecting cell clones at this stage, because the purpose of the experiment was to establish a full-length HCV RNA replicating cell line.

### 3-2 Drug Sensitivity of Cell Line

Sensitivity of an HCV replicon in sOL cells was examined with respect to drugs reported to have anti-HCV activity in HuH-7 cell-derived cells. It was also investigated whether treatment with such drugs would enable production of sOL cured cells (cells expected to have an intracelluler environment suited for HCV RNA replication) considered to be necessary for the production of full-length HCV RNA replicating cells. Note that HuH-7 cell-derived sO cells were compared with sOL cells in an experiment conducted to compare effects.

A 6-well plate was inoculated with 1×10⁵ cells, and various drugs were added 24 hours later. The drugs were added so that the final concentration was 20 IU/ml for IFN-α, IFN-β, and IFN-γ, 5 μM for fluvastatin (FLV), and 0.5 μg/ml for Cyclosporin A (CsA). After 5 days, the expression levels of NS5B protein in the cells were analyzed by Western blotting. As shown in FIG. 3(b), the sOL cells had about the same level of sensitivity as the sO cells to each anti-HCV agent. It was therefore considered possible to obtain cured sOL cells by treatment with these drugs.

### 4: Full-Length HCV RNA Replicating Cell Line

### 4-1 Preparation of Cell Line

IFN-γ (10³ IU/ml) was added to sOL cells five times at 4-day intervals in the absence of G418 to obtain cured cells OLc from which HCV-O strain replicon RNA was excluded. Note that the resulting cells were identified as cured cells by the absence of HCV RNA, and by the lack of expression of the proteins encoded by the HCV genome.

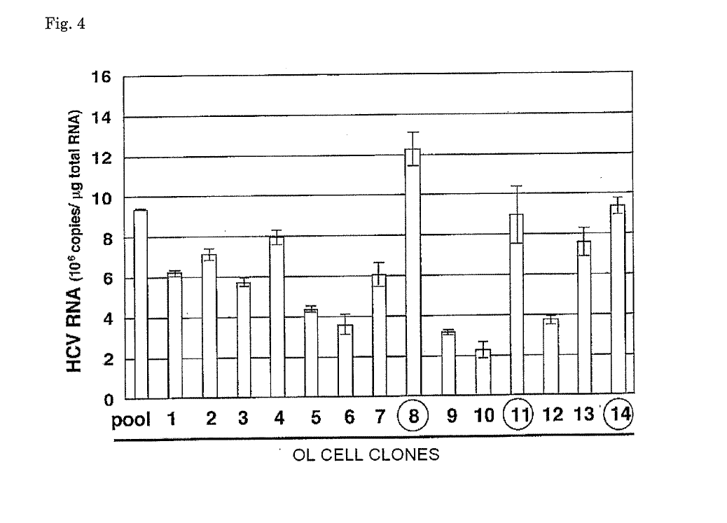

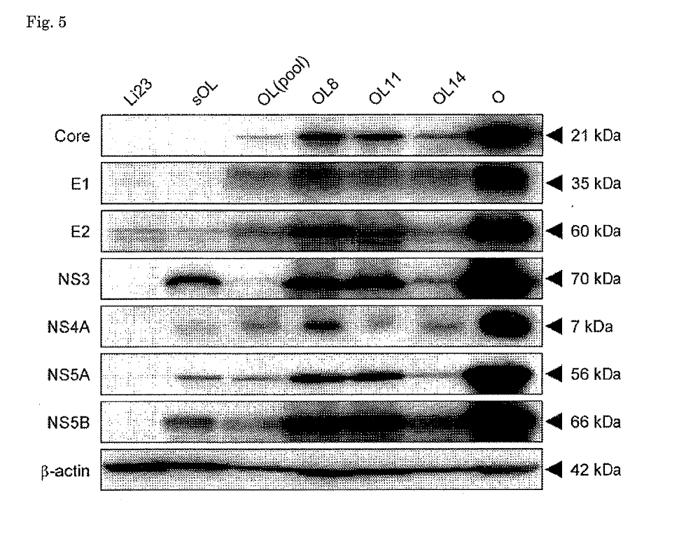

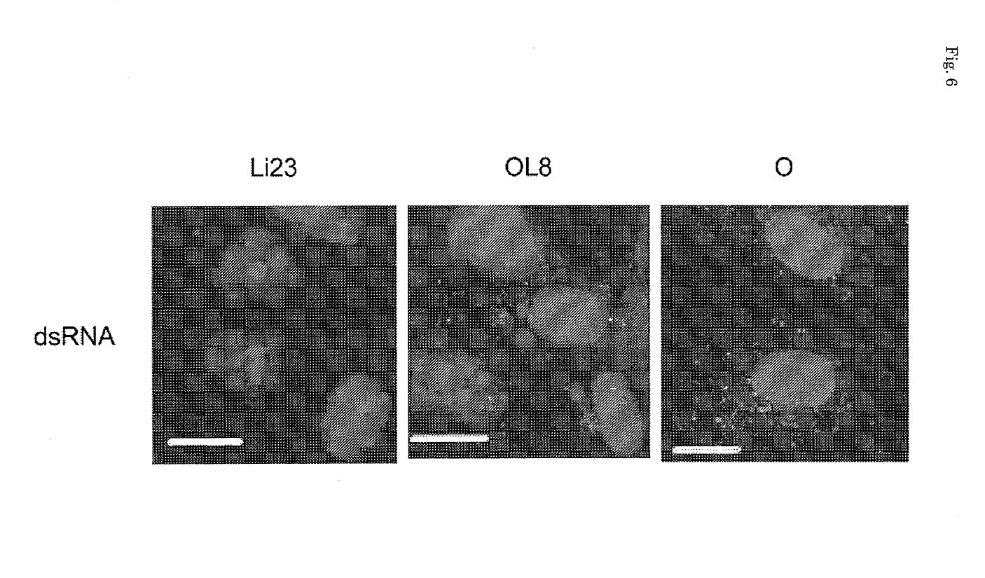

The RNA (2 μg or 4 μg) synthesized in vitro from pON/C-5B/QR, KE, SR was introduced to sOLc cells (8×10⁶) according to the method described in Non-Patent Document 2. After 2 days, the medium was replaced with a medium that contained G418 (0.3 mg/μl) and NaHCO₃ (0.15%). The cells were cultured for 3 weeks with medium replacement every 4 days. As a result, G418-resistant colonies were obtained at each RNA level (the results are not presented). Some of the colonies (1 plate) were stained with CBB. The number of stained colonies was counted, and the rate of colony formation per 1 μg RNA was calculated to be about 100 colonies/μg RNA. Considering the possibility of incomplete drug selection by G418 and the possibility of minute amounts of remaining HCV-O strain replicon, large colonies were selected for cloning, and cells that showed sustained high levels of full-length HCV RNA replication were obtained as G418-resistant colonies (OL1 to OL14). By the quantitative comparison of HCV RNA in the cells using LightCycler PCR, the top three clones (OL8 cells, OL11 cells, and OL14 cells) were selected, and used for HCV protein detection and HCV gene analysis (FIG. 4). About 200 remaining colonies were mixed, and used as OL (pool) cells for further analysis. Note that replication of 11-kb full-length HCV RNA and the absence of 8-kb replicon RNA in the OL cells were confirmed in all OL cell clones and in OL (pool) cells (the results are not presented). It was also confirmed from the result of Western blotting for various HCV proteins that OL8, OL11, and OL14 cells expressed proteins in amounts considered to be sufficient for various experiments, though the expression levels were lower than that in 0 cells (FIG. 5). Note that OL14 cells had lower expression levels of HCV proteins than OL8 cells or OL11 cells. Further, PCR conducted for the HCV 5′ UTR confirmed the lack of HCV genome incorporation in the host genome in OL8, OL11, and OL14 cells (the results are not presented).

Subsequently, OL8 cells were observed using an immunofluorescent technique with anti-dsRNA antibodies, in order to detect double-stranded RNA (dsRNA), a replication intermediate of HCV RNA, in the full-length HCV RNA-replicating OL8 cells. The O cells and Li23 cells were used as positive control and negative control, respectively.

The cells cultured for 4 days after being inoculated on a collagen-coated cover slip were fixed at room temperature with a PBS solution containing 3% paraformaldehyde, and treated with 0.1% Triton X-100 to prepare permeable cells. After treatment with 1% (v/v) bovine serum albumin (BSA), the cells were treated with anti-dsRNA antibodies (K1: English and Scientific Consulting; primary antibodies), and then with Cy2-conjugated anti-mouse antibodies (Jackson Immuno Research, West Grove; secondary antibodies). The cell nuclei were stained with 4′,6-diamidino-2-phenylindole (Sigma). The cover slip was placed on a glass slide using a PermaFluor Aqueous Mountant (ThermoFisher), and observed with a confocal laser scanning microscope (LSM510; Carl Zeiss). Photographed images are shown in FIG. 6 (bar length, 20 μm).

As shown in the figure, small dot-like fluorescence scattered over the cytoplasm was observed in the OL8 and O cells. Li23 cells did not show any such fluorescence. The fact that the replication intermediate double-stranded RNA was observed in OL8 cells as in O cells can be taken as evidence of efficient HCV RNA replication in the OL8 cells.

### 4-2 Drug Sensitivity of Cell Lines

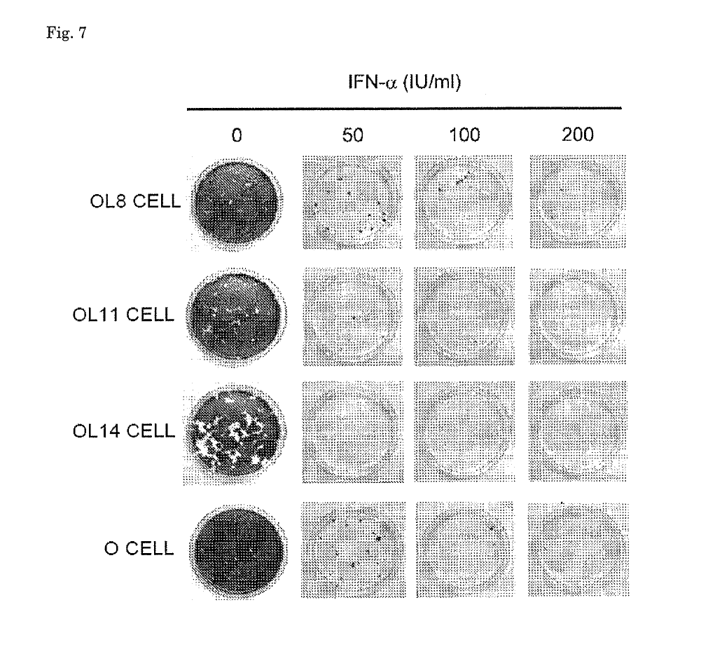

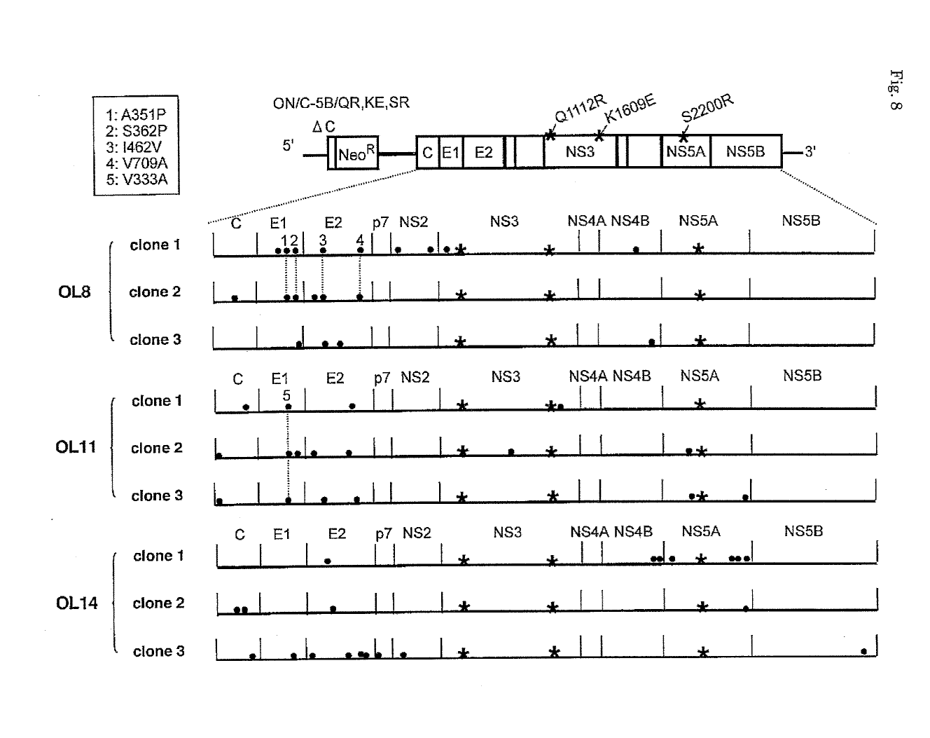

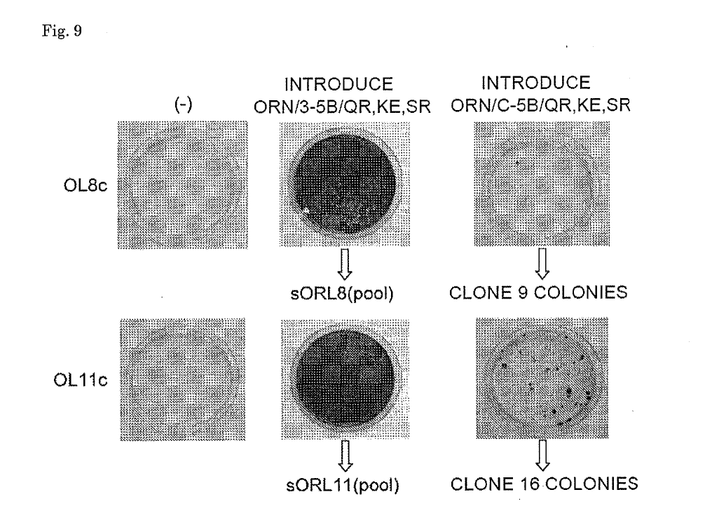

Whether IFN-α suppresses the full-length HCV RNA replication in OL8, OL11, and OL14 cells was determined by colony assay. Cells (1×10⁴) were inoculated on dishes having an outer diameter of 10 cm, and cultured for 25 days in the presence of G418 (0.3 mg/ml) while adding IFN-α (0, 50, 100, or 200 IU/ml) every 4 days. The colonies that appeared as G418 resistant cells were stained with CBB solution (FIG. 7). Some G418-resistant cell colonies were obtained in the OL8 and OL11 cells; however, the number did not differ much from that obtained in the O cells used as control. No G418-resistant cell colonies were obtained in OL14 cells. The results demonstrated that the full-length HCV RNA replication in the OL8, OL11, and OL14 cells were highly sensitive to IFN-α as in O cells.

### 4-3 HCV Gene Analysis in Cell Lines

The presence or absence of new adaptive mutations other than the three adaptive mutations (Q1112R, K1609E, and S2200R) introduced in the full-length HCV RNA replicated in the OL8, OL11, and OL14 cells was examined. Total RNA was prepared from each cell line, and the full-length HCV RNA was amplified according to the RT-PCR method described in Non-Patent Document 7 (from the 5′ UTR-NS2 5.1-kb first half to the NS3-NS5B 6-kb second half). The RT primer 290ROK was used for the amplification of the first half, and a primer set (21X and NS3RXOK) was used for PCR. The RT primer 386R was used for the amplification of the second half, and a primer set (NS2XOK and 9388RX) was used for PCR. A Primscript (Takara) was used for RT, and a KOD-plus DNA polymerase (Toyobo) for PCR. The amplification product was inserted into a plasmid vector (pBR322MC), and the base sequence of the inserted portion was determined, and compared with the base sequence of ON/C-5B/QR, KE, SR originally introduced into the cells (FIG. 8).

As a result, the following became clear. Note that no mutation was recognized in the 5′ UTR (341 bases) base sequences of the analyzed 9 clones, though not shown in the figure.

(1) The mutations Q1112R, K1609E, and S2200R originally introduced were conserved in the total of 9 clones analyzed (3 clones for each cell line).

(2) The NS3 to NS5B region essential for the HCV RNA replication did not contain any new mutation that accompanied amino acid substitutions conserved in the three clones derived from each cell line. The mutations were all clone specific. The number of mutations in the NS3 to NS5B region per clone was low: 1 in OL8 cells, 1.3 in OL11 cells, and 2.6 in OL14 cells. The clone specific mutations (Q1067R, K1397R, I1612M, V1864A, V1929L, E1937D, C1968W, T1989S, T2169A, L2171P, P2322L, T2332A, S2380P, and W2404R) detected in the NS3 to NS5B region did not classify as any of the adaptive mutations reported thus far. However, a previous report (Lohmann et al., JVI, 77: 3007-3019, 2003) indicates that the Q2933R detected in clone 3 of OL14 cells is a weak adaptive mutation (1.6-fold increase in replication level). It was therefore considered that the Q1112R, K1609E, and S2200R mutations were essential for the full-length HCV RNA replication in OL cell lines, and that no additional adaptive mutation was necessary.

(3) On the other hand, relatively larger numbers of mutations were observed in the core to NS2 region. In OL11 cells, a mutation (V333A) accompanied by a common amino acid substitution across the three clones was detected in the E1 region. In OL8 cells, no common mutation was detected in the three clones; however, four mutations (A351P and S362P in the E1 region, and 462V and V709A in the E2 region) were detected and recognized in two of the three clones. The number of mutations in the core to NS2 region per clone was higher than in the NS3 to NS5B region: 5.3 in the OL8 cells, 4 in the OL11 cells, and 4 in the OL14 cells. The results therefore suggested that this region was not necessary for HCV RNA replication.

Neighbour-joining analysis was performed with GENETYX-MAC (Software Development) for the three clones derived from OL8, OL11, and OL14 cells, using the base sequence and amino acid sequence in the HCV polyprotein, and a genetic phylogenetic tree was created based on the parental clone ON/C-5B/QR, KE, SR (the results are not presented). The phylogenetic tree appeared the same at the base sequence level or the amino acid sequence level. However, the OL14 cells did not form genetically-independent clusters. As described above, OL14 cells had considerably lower HCV protein expression levels than the other two cells, and as such the OL8 cells and OL11 cells were used for the subsequent analyses.

### 5: Cell Line Capable of Replicating Reporter Gene-Containing HCV Replicon RNA or Full-Length HCV RNA

### 5-1 Preparation of Cell Lines

Cured cells OLc required for JFH1 strain HCV infection experiment were prepared by adding IFN-γ (10³ IU/ml) five times at 4-day intervals to the three clones of the OL cells (OL8, OL11, and OL14 cells), and to other clones (OL cells) in the absence of G418. About 4 to 5×10⁵ OL8 cells and OL11 cells were inoculated on dishes having an outer diameter of 10 cm, and IFN-γ (10³ IU/ml) was added four times at 4-day intervals. The cells were appropriately subcultured when the dish became full. The cells subcultured after the 4th addition of IFN-γ were divided into two groups of dishes, one containing medium supplemented with G418 (0.3 mg/ml) and NaHCO₃ (0.15%), and one continuously used to culture the cells with the medium alone. IFN-γ was then added once to each group, and the cells were cultured for at least 4 days before stained with CBB. While the cells cultured in the G418-free medium grew and filled the dish, the cells were completely killed when cultured in the medium supplemented with G418 (the results are not presented).

Cell lines capable of replicating reporter gene-carrying HCV RNA (replicon or full-length) were prepared using OL8c cells and OL11c cells. The RNAs (10 μg; ORN/3-5B/QR, KE, SR, or 20 μg; ORN/C-5B/QR, KE, SR) synthesized in vitro from pORN/3-5B/QR, KE, SR and pORN/C-5B/QR, KE, SR were introduced into OL8c cells or OL11c cells (8×10⁶) according to the method described in Non-Patent Document 2. After 2 days, the medium was replaced with a medium containing G418 (0.3 mg/ml) and NaHCO₃ (0.15%). The medium was replaced every 4 days, and the cells were cultured for 2 to 3 weeks.

In the ORN/3-5B/QR, KE, SR (replicon RNA)-introduced OL8c and OL11c cells, G418-resistant cells filled the dish in 2 weeks (FIG. 9). Because the whole cells were estimated to be several tens of thousands of colonies, the G418-resistant cells were mixed without cloning, and used as reporter gene-carrying replicon-replicating cells (sORL8 (pool) cells and sORL11 (pool) cells). These cells had luciferase activities measuring 8×10⁵ and 15×10⁵, respectively, in terms of actual measurement values per 2×10⁵ cells (Promega assay kit). It was therefore found that the both of these cells were sufficient for the activity evaluation of anti-HCV agents. Note that the doubling time of the cells using a common subculture medium (in the presence of G418) was calculated as 53 hours and 40 hours. It was also confirmed that the replicon RNA in these cells were not incorporated into the host DNA (the results are not presented).

In the ORN/C-5B/QR, KE, SR (full-length HCV RNA)-introduced cells, only small numbers of G418-resistant colonies appeared even after about 3 weeks from the introduction, about 30 from OL8c cells, and about 400 from OL11c cells. Nine cell colonies were cloned from OL8c cells (ORL8-1 to ORL8-9), and 16 cell colonies were cloned from OL11c cells (ORL11-1 to ORL11-16) (FIG. 9). The remaining uncloned cell colonies from OL11c cells were mixed, and used as ORL11 (pool) cells. The measured luciferase activity of the ORL11 (pool) cells was 7×10⁵ (Promega assay kit). The cells were therefore found to be sufficient for the activity evaluation of anti-HCV agents. The doubling time of the cells using a common subculture medium (in the presence of G418) was calculated as 44 hours. It was also confirmed that the HCV genome in the cells was not incorporated into the host DNA (the results are not presented).

The expression level of HCV core protein in the ORL11 (pool) cells was determined by Western blotting. The result suggested that the expression level of core protein in the ORL11 (pool) cells was considerably lower than that in OR6 cells. It was therefore considered that selection of cloned cells having higher expression levels was necessary.

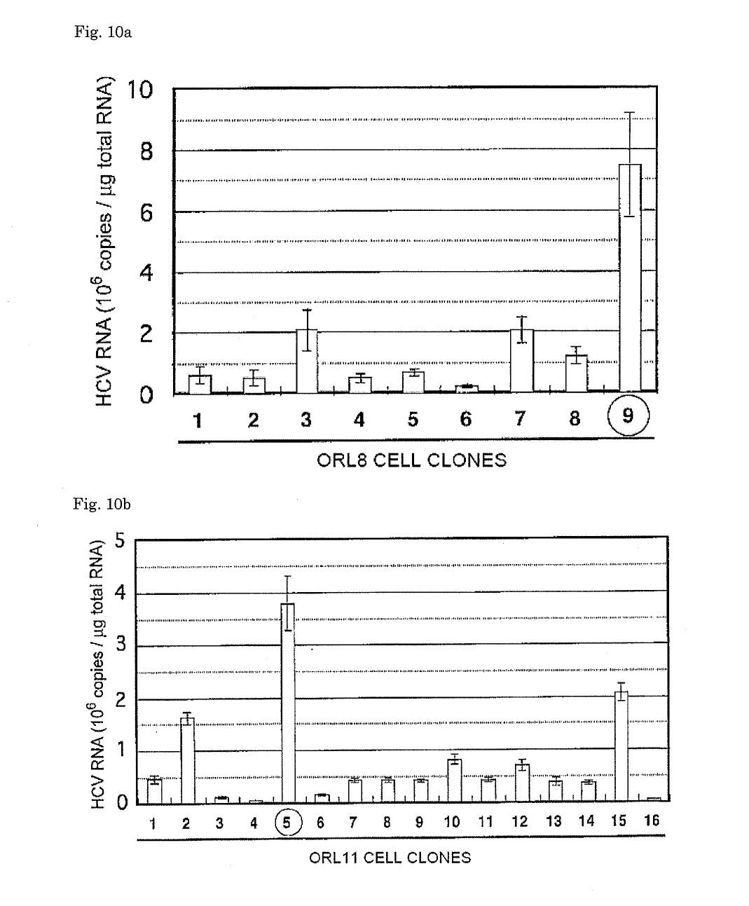

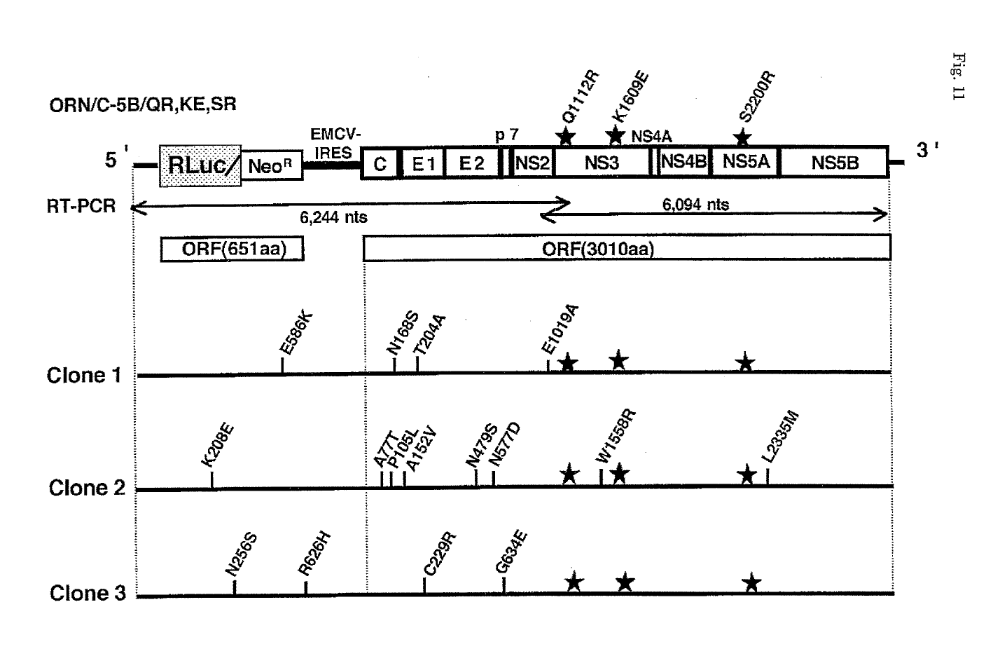

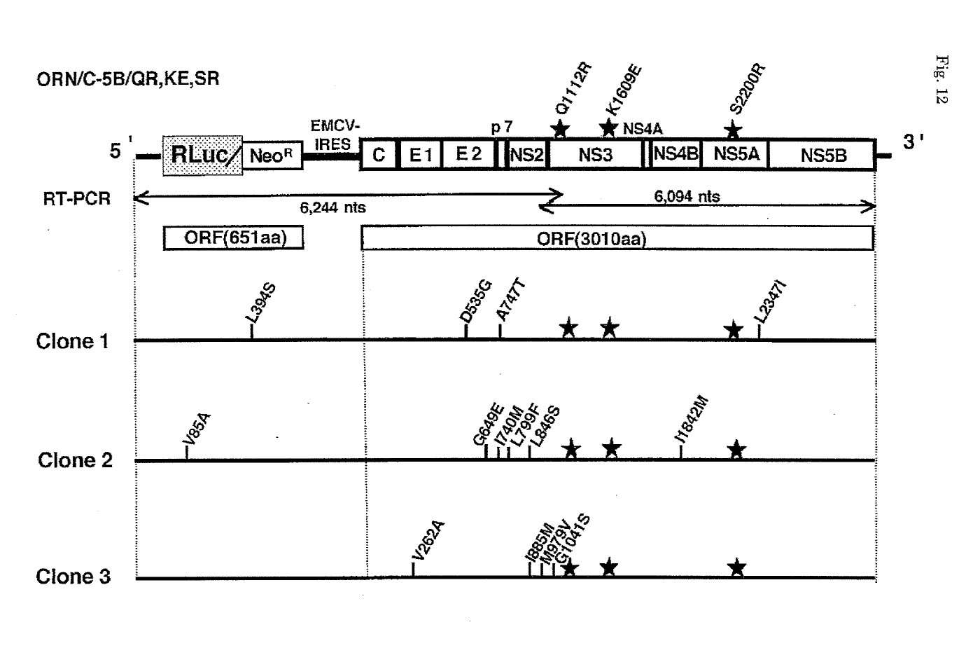

Cells that showed sustained high levels of RL gene-carrying full-length HCV RNA replication were obtained as G418-resistant colonies (FIG. 10(a) and FIG. 10(b)). Specifically, the top two clones (ORL8-9 cells and ORL11-5 cells; referred to as ORL8 cells and ORL11 cells, respectively) were selected by the quantitative comparison of the HCV RNAs in the cells using LightCycler PCR. The luciferase reporter assays of these cells are considered useful for the screening and evaluation of various anti-HCV agents.

### 5-2 HCV Gene Analysis in Cell Lines

The presence or absence of new adaptive mutations other than the three adaptive mutations (Q1112R, K1609E, and S2200R) originally introduced to the reporter gene-carrying full-length HCV RNA replicated in the ORL8 cells was examined. Total RNA was prepared from ORL8 cells, and the full-length HCV RNA was amplified according to the RT-PCR method described in Non-Patent Document 7 (from the 5′ UTR-NS2 6.2-kb first half to the NS3-NS5B 6.1-kb second half). The RT primer 290ROK was used for the amplification of the first half, and a primer set (21X and NS3RXOK) was used for PCR. The RT primer 386R was used for the amplification of the second half, and a primer set (NS2XOK and 9388RX) was used for PCR. A Primscript (Takara) was used for RT, and a KOD-plus DNA polymerase (Toyobo) for PCR. The amplification product was inserted into a plasmid vector (pBR322MC), and the base sequence of the inserted portion was determined, and compared with the base sequence of ORN/C-5B/QR, KE, SR originally introduced into the cells (FIG. 11). As a result, the following became clear.

(1) The mutations Q1112R, K1609E, and S2200R originally introduced were conserved in the three clones analyzed.

(2) New mutations that accompanied amino acid substitutions conserved in the three clones were not detected in the 5′ UTR to NS5B region for which the base sequence was determined. All new mutations accompanied by amino acid substitutions (4 in clone 1; 8 in clone 2; and 4 in clone 3) were specific to the clones analyzed. It was therefore considered that the Q1112R, K1609E, and S2200R mutations were essential for the reporter gene-carrying full-length HCV RNA replication in the ORL8 cells, and that no additional adaptive mutation was necessary.

(3) Mutations that accompanied amino acid substitutions were found in larger numbers in the core to NS2 region than in the NS3 to NS5B region. The same phenomenon was also seen in the results from the full-length HCV RNA replicating OL8 cells, OL11 cells, and OL14 cells.

(4) The clone specific mutations W1558R and L2335M detected in the NS3 to NS5B region did not classify as any of the adaptive mutations reported thus far.

The presence or absence of new adaptive mutations other than the three adaptive mutations (Q1112R, K1609E, and S2200R) originally introduced to the reporter gene-carrying full-length HCV RNA replicated in the ORL11 cells was determined in the same manner as in ORL8 cells (FIG. 12). As a result, the following became clear.

(1) As for the ORL8 cells, the mutations Q1112R, K1609E, and S2200R originally introduced were conserved in the three clones analyzed.

(2) New mutations that accompanied amino acid substitutions conserved in the three clones were not detected in the 5′ UTR to NS5B region for which the base sequence was determined. All new mutations accompanied by amino acid substitutions (4 in clone 1; 6 in clone 2; and 4 in clone 3) were specific to the clones analyzed. It was therefore considered that the Q1112R, K1609E, and S2200R mutations were essential for the reporter gene-carrying full-length HCV RNA replication in the ORL11 cells, and that no additional adaptive mutation was necessary.

(3) Mutations that accompanied amino acid substitutions were found in larger numbers in the core to NS2 region than in the NS3 to NS5B region. The same phenomenon was also seen in the results from the full-length HCV RNA replicating OL8 cells, OL11 cells, and OL14 cells.

(4) The clone specific mutations G1041S, 11842M, and L2347I detected in the NS3 to NS5B region did not classify as any of the adaptive mutations reported thus far.

### 5-3 Cell Line Behavior Analysis

ORL8, ORL11, and OR6 cells were each inoculated in three wells of a 24-well plate, each well containing 2×10⁴ cells (1-ml medium: assay medium containing no G418, fungizone, or NaHCO₃). The cells were cultured for 4 days, and luciferase activity was measured. The measured values were, on average, 1×10⁶ in ORL8 cells, 2×10⁶ cells in ORL11 cells, and 2×10⁶ cells in OR6 cells (the results are not presented). This level of luciferase activity was considered sufficient for experiments that study effectiveness after addition of anti-HCV agents. The value was about 100 in OL8c cells and OL11c cells used as controls.

To examine the doubling time of the cells, the cells were inoculated under the conditions of the assay system used to measure luciferase activity, and the cells after 24, 48, 72, and 96 hours were counted using a trypan blue staining technique. Measurements were made in 3 wells at each point, and a mean value was determined for the measurement of doubling time in a logarithmic growth phase. For comparison and contrast, the OR6 cells were also counted in the same manner. The doubling time of cells were 23 hours in ORL8 cells, 26 hours in ORL11 cells, and 34 hours in OR6 cells (the results are not presented).

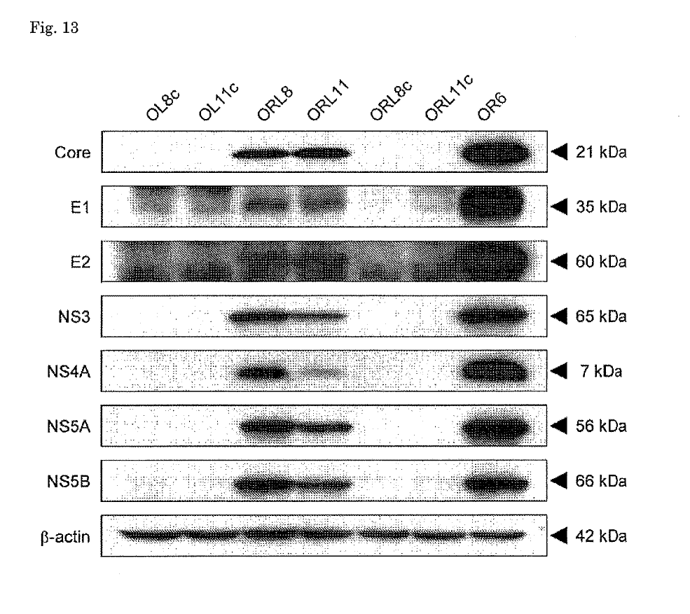

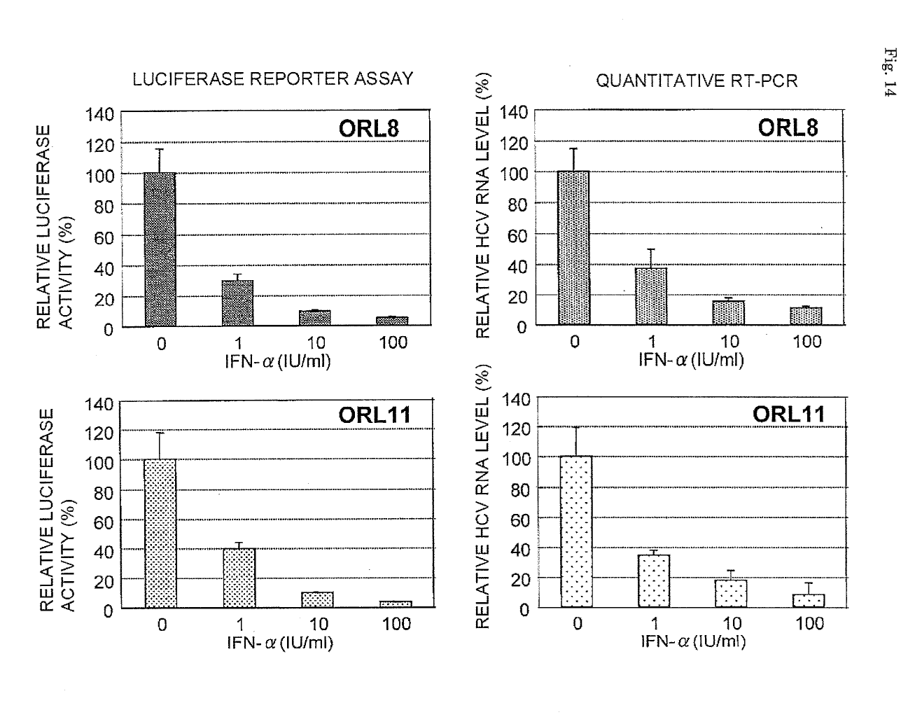

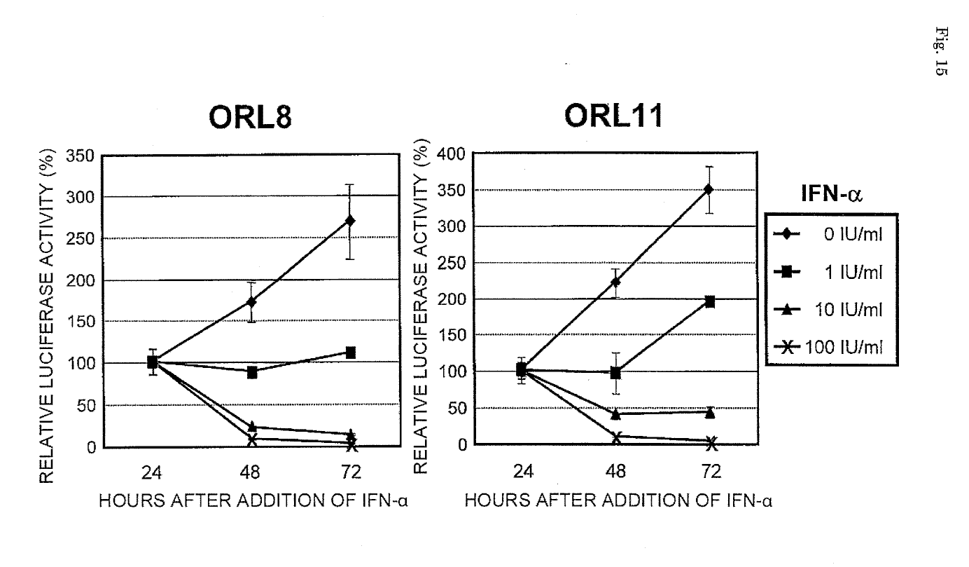

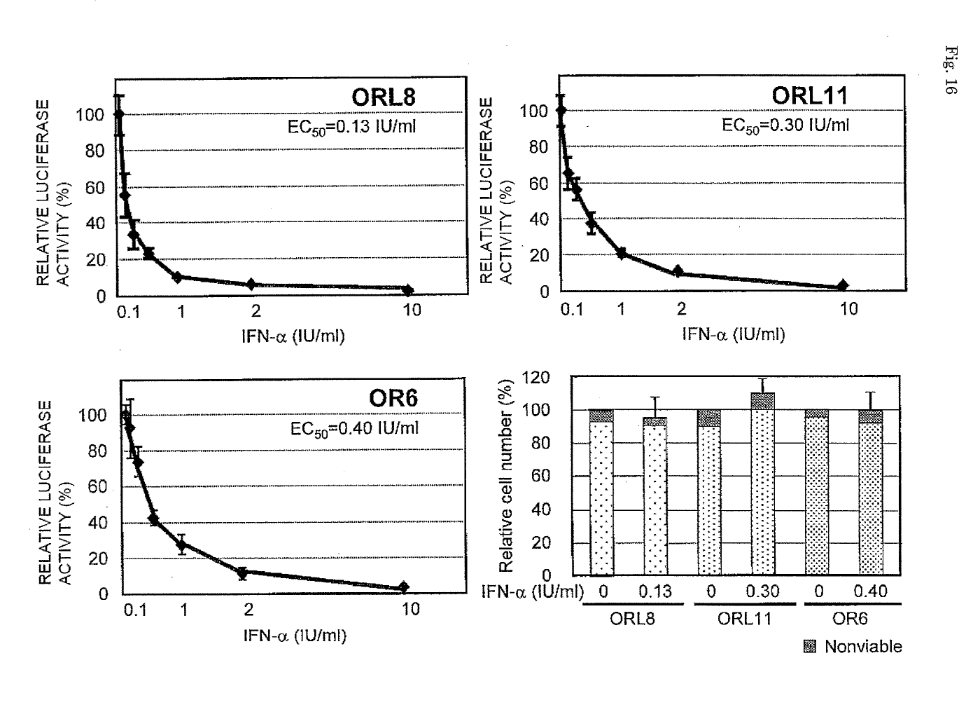

The expression level of each HCV protein (core, E1, E2, NS3, NS4A, NS5A, and NS5B) in the ORL8 and ORL11 cells was compared with that in the OR6 cells using Western blot analysis (FIG. 13). As controls, the same analysis was also made for the cured OL8c, OL11c, ORL8c, and ORL11c cells. β-actin detection using β-actin antibody was also performed to find the amounts of proteins used in the analysis. The expression level of each HCV protein in ORL8 and ORL11 cells was considerably lower than that in OR6, but the expression level was considered sufficient for various analyses. The lower expression levels relative to OR6 cells was in accord with the lower HCV RNA levels of ORL8 and ORL11 cells in comparison with OR6 cells (>1×10⁷ copies/mg total RNA; FIGS. 10(a) and 10(b)).

### 5-4 Drug Sensitivity of Cell Lines

Analysis was made as to the usefulness of the cell lines ORL8 and ORL11 for the evaluation of drug effects, as in OR6 cells (see Patent Document 1). As described in Patent Document 1, there is a correlation between cell-derived luciferase activity and HCV RNA level in OR6 cells, making the OR6 cells a convenient cell line for the evaluation of drug effects.

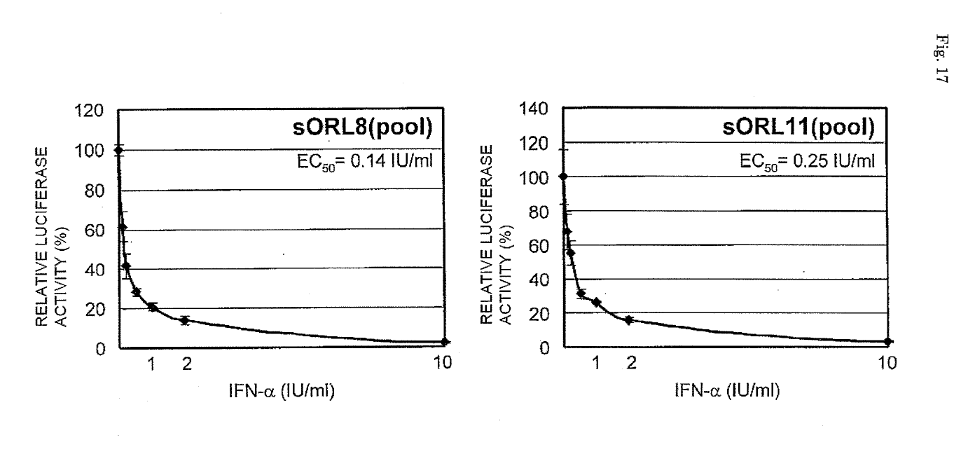

The results are shown in FIG. 14. The graphs on the left represent the measured luciferase activities 24 hours after the addition of IFN-α (0, 1, 10, 100 IU/ml) to the ORL8 and ORL11 cells (each cultured for 1 day after inoculating 2×10⁴ cells in a 24-well plate). Measurements were made in at least 3 wells at each point. SD values are also shown in the figure. One hundred percent measurement values of 400,000 and 500,000 were obtained for the ORL8 cells and ORL11 cells, respectively. The graphs on the right represent the results of quantitative HCV RNA measurements by LightCycler PCR 24 hours after the addition of IFN-α (0, 1, 10, 100 IU/ml) to the ORL8 and ORL11 cells (each cultured for 2 days after inoculating 2×10⁵ cells in a 6-well plate). Measurements were made in at least 3 wells at each point. SD values are also shown in the figure. As represented in the figure, the luciferase activity and the HCV RNA level decreased in a manner that depended on IFN-α concentration, and the results of these measurements had a good correlation as did the result from OR6 cells. The results thus demonstrated that the ORL8 and ORL11 cells were useful for the quantification of HCV RNA replication level with a simple luciferase assay.

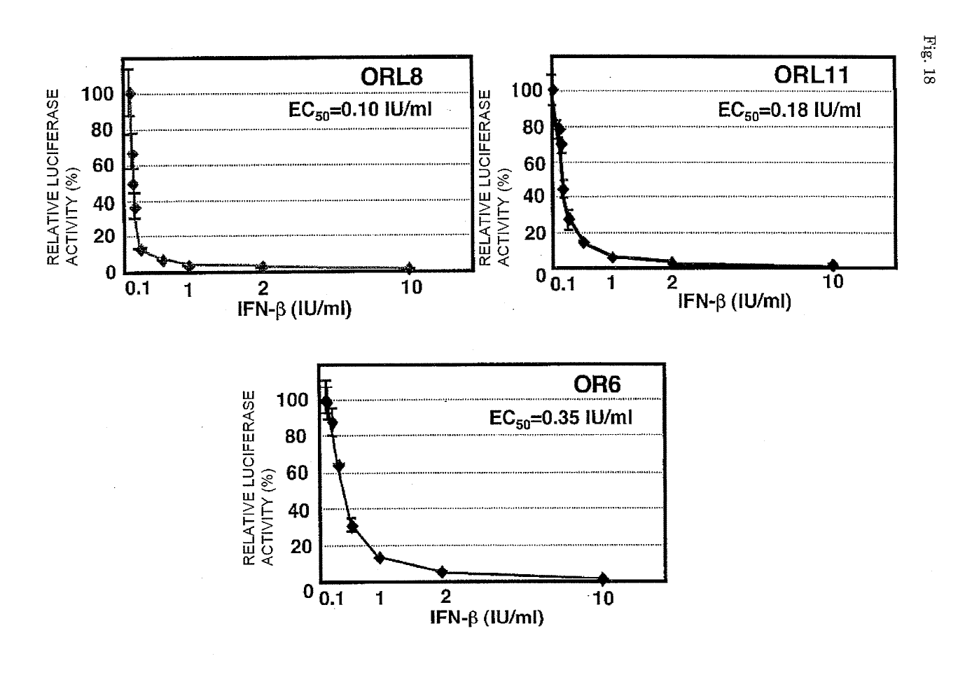

Time-dependent viral effect is continued in OR6 cells (Naka et al., BBRC, 330: 871-879, 2005). It was investigated whether similar viral effects also can be seen in ORL8 and ORL11 cells (FIG. 15). Cells (2×10⁴) were inoculated on a 24-well plate, and the predetermined quantities of IFN-α (0, 1, 10, 100 IU/ml) were added 24 hours later. Luciferase activity of each cell line was then measured at hour 24, 48, and 72. A separately prepared Li23 cell medium containing 10% FBS was used for the dilution of IFN-α. SD values were calculated based on the results from three samples at each point.

From the reference luciferase value of 100 at twenty-four hours after the addition of IFN-α, the activity showed no change up until hour 48 in response to the addition of 1 IU/ml IFN-α. At hour 72, a slight increase was observed in ORL8 cells, and a clear increase again in ORL11 cells. These results indicate the high HCV RNA replication levels of these cells. However, the reincrease after 72 hours was not so evident in cells treated with 10 IU/ml or 100 IU/ml IFN-α. Further, a decrease in luciferase activity, and the strong anti-HCV effect of IFN-α were observed as early as 24 hours after the addition of IFN-α, though not presented in the figure. The measured luciferase activity values were 380,000 (no addition of IFN-α), 113,000 (addition of 1 IU/ml IFN-α), 36,000 (addition of 10 IU/ml IFN-α), and 19,000 (addition of 100 IU/ml IFN-α) for the ORL8 cells. Because the luciferase activity 72 hours after the addition of 1 IU/ml IFN-α is 50% or less of the luciferase activity obtained without IFN-α, EC₅₀ (50% effective drug concentration) is estimated to be 1 IU/ml or less. The measured values for the ORL11 cells were 750,000 without addition of IFN-α, 300,000 with 1 IU/ml IFN-α, 76,000 with 10 IU/ml IFN-α, and 27,000 with 100 IU/ml IFN-α. As in the ORL8 cells, the luciferase activity 72 hours after the addition of 1 IU/ml IFN-α is 50% or less of the luciferase activity obtained without IFN-α, and thus EC₅₀ (50% effective drug concentration) is estimated to be 1 IU/ml or less.

Thus, at least for IFN-α, the results suggested the potential of ORL8 or ORL11 cells as a convenient assay system capable of monitoring the HCV RNA replication level solely by the measurement of luciferase activity 72 hours after the addition of the drug, as also suggested for OR6 cells.

### 5-5 Anti-HCV Effect of Various Drugs

Drugs reported to have anti-HCV activities were evaluated using an ORL8- or ORL11-cell assay system. An OR6-cell assay system was used as a control. Note that, depending on compounds, use of DMSO or ethanol as a solvent is needed; however, it has been confirmed that the assay system is not affected as long as the DMSO concentration is 0.5% or less, and that the ethanol concentration is 0.2 to 0.25% (data not presented).

### (A) IFN-α

IFN-α (0, 0.1, 0.2, 0.5, 1, 2, 10 IU/ml: Sigma, I2396) was added to each cell line, and luciferase activity after 72 hours was measured. By analysis, the EC₅₀ values of the ORL8, ORL11, and OR6 cells were calculated as 0.13 IU/ml, 0.30 IU/ml, and 0.40 IU/ml, respectively. Values close to these were obtained in the same experiment repeated three times, yielding good reproducibility. The ORL8 cells had the highest sensitivity for IFN-α, followed by ORL11 cells and OR6 cells. Representative results are presented in FIG. 16.

In order to ascertain that the decrease in luciferase activity in response to the addition of IFN-α was not due to the cell growth inhibition or cytotoxicity by IFN-α, the cells were cultured under the same conditions used for the luciferase assay, and counted using a trypan blue staining technique. The effect of adding IFN-α in the concentration corresponding to the EC₅₀ value of each cell line was examined. The results for all cells were 95% or higher over the control cells, and cytotoxicity by IFN-α was hardly recognized.

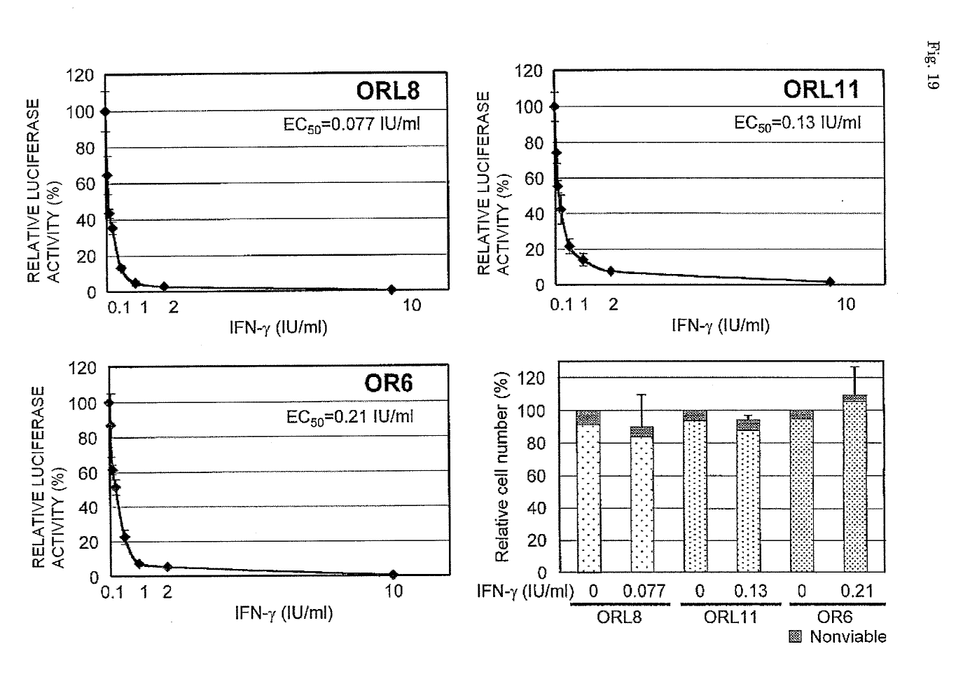

The same experiments were conducted using sORL8 (pool) cells and sORL11 (pool) cells. By analysis, the EC₅₀ values of sORL8 (pool) cells and sORL11 (pool) cells were calculated as 0.14 IU/ml and 0.25 IU/ml, respectively, about the same values obtained from ORL8 and ORL11 cells (0.13 IU/ml and 0.30 IU/ml, respectively). The results suggest that the influence of IFN-α does not differ greatly for the replication of HCV replicon RNA and full-length HCV RNA. Representative results are presented in FIG. 17.

### (B) IFN-β

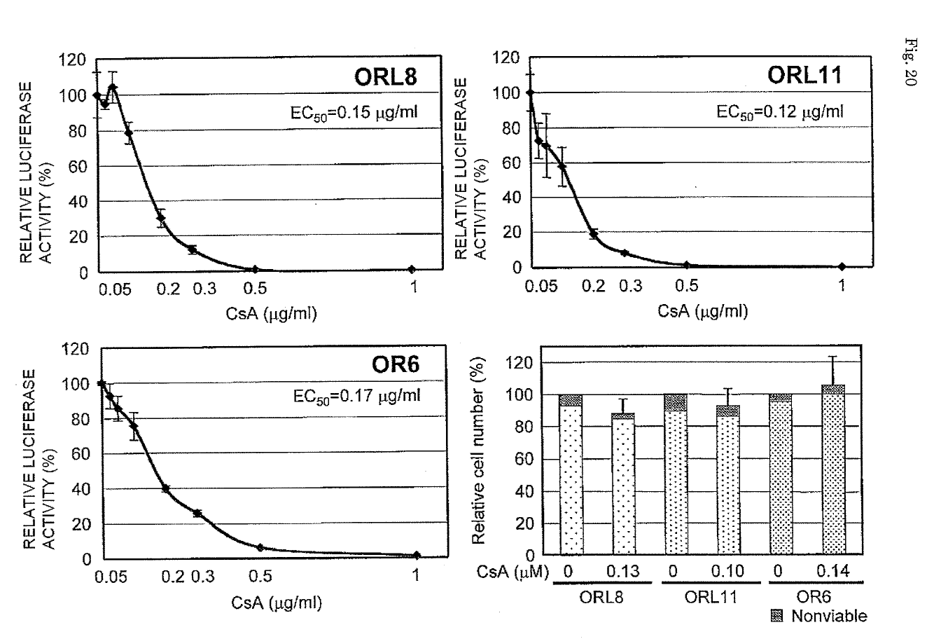

IFN-β(0, 0.05, 0.1, 0.2, 0.5, 1, 2, 10 IU/ml: provided by Toray) was added to each cell line, and luciferase activity after 72 hours was measured. By analysis, the EC₅₀ values of ORL8, ORL11, and OR6 cells were calculated as 0.10 IU/ml, 0.18 IU/ml, and 0.35 IU/ml, respectively. Representative results are presented in FIG. 18. The trend seen in IFN-α was also observed in IFN-β, with the ORL8 cells showing the highest sensitivity.

### (C) IFN-γ

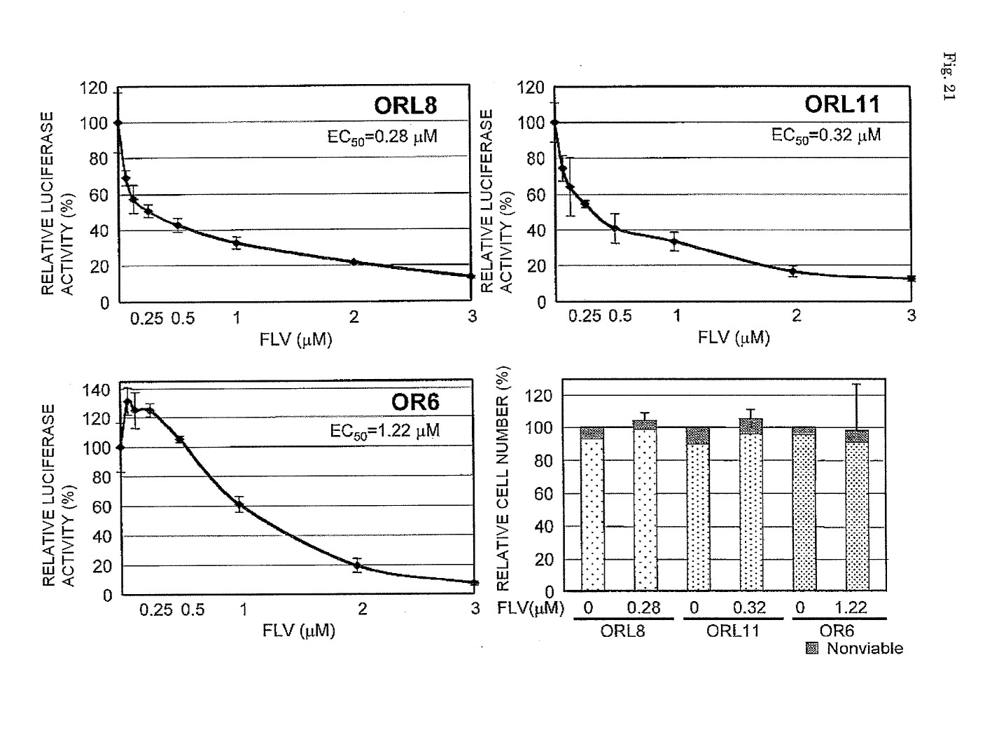

IFN-γ (0, 0.05, 0.1, 0.2, 0.5, 1, 2, 10 IU/ml: Sigma, 11520) was added to each cell line, and luciferase activity after 72 hours was measured. By analysis, the EC₅₀ values of ORL8, ORL11, and OR6 cells were calculated as 0.077 IU/ml, 0.13 IU/ml, and 0.21 IU/ml, respectively. Representative results are presented in FIG. 19. The same trend seen in IFN-α or IFN-β was also observed in IFN-γ, with the ORL8 cells showing the highest sensitivity.

In order to ascertain that the decrease in luciferase activity in response to the addition of IFN-γ was not due to the cell growth inhibition or cytotoxicity by IFN-γ, the cells were cultured under the same conditions used for the luciferase assay, and counted using a trypan blue staining technique. The effect of adding IFN-γ in the concentration corresponding to the EC₅₀ value of each cell line was examined. The results for all cells were 90% or higher over the control cells, and cytotoxicity by IFN-γ was hardly recognized.

### (D) Cyclosporin A (CsA)

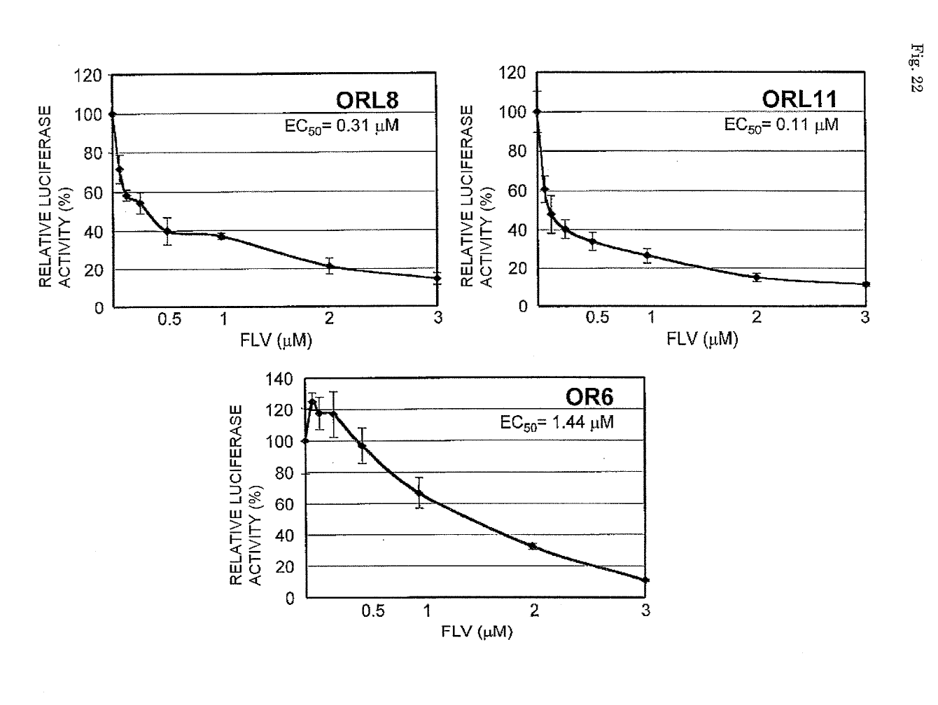

CsA (0, 0.025, 0.05, 0.1, 0.2, 0.3, 0.5, 1 μg/ml: Sigma, C3662) was added to each cell line, and luciferase activity after 72 hours was measured. By analysis, the EC₅₀ values of the ORL8, ORL11, and OR6 cells were calculated as 0.15 μg/ml, 0.12 μg/ml, and 0.17 μg/ml, respectively. Representative results are presented in FIG. 20. Unlike IFN-α, IFN-β, or IFN-γ, the sensitivity of the ORL11 cells to CsA was only slightly higher than those of the other cells, and the differences between the three were smaller than those observed in IFNs.

In order to ascertain that the decrease in luciferase activity in response to the addition of CsA was not due to the cell growth inhibition or cytotoxicity by CsA, the cells were cultured under the same conditions used for the luciferase assay, and counted using a trypan blue staining technique. The effect of adding CsA in the concentration corresponding to the EC₅₀ value of each cell line was examined. The results for all cells were 87% or higher over the control cells, and cytotoxicity by CsA was hardly recognized.

### (E) Fluvastatin (FLV)

FLV (0, 0.063, 0.125, 0.25, 0.5, 1, 2, 3 μM: Calbiochem, 344095) was added to each cell line, and luciferase activity after 72 hours was measured. By analysis, the EC₅₀ values of the ORL8, ORL11, and OR6 cells were calculated as 0.28 μM, 0.32 μM, and 1.22 μM, respectively. Representative results are presented in FIG. 21.

The anti-HCV effect of FLV is found with an assay using OR6 cells (see Patent Document 1). The ORL8 and ORL11 cells had EC₅₀ values (0.28 μM and 0.32 μM, respectively) considerably smaller than the EC₅₀ value (1.22 μM) of OR6 cells. There is an increasing trend for clinical trials that additionally use FLV in the PEG-IFN+ribavirin combination therapy, and these tests are producing good results (Sezaki et al. Kanzo, 49: 22-24, 2008). The effectiveness of FLV in clinical trials provide supportive evidence for the credibility of the EC₅₀ values of FLV obtained in the cell assay systems using ORL8 and ORL11 cells, and support the usefulness of the cell assay systems that use ORL8 and ORL11 cells.

In order to ascertain that the decrease in luciferase activity in response to the addition of FLV was not due to the cell growth inhibition or cytotoxicity by FLV, the cells were cultured under the same conditions used for the luciferase assay, and counted using a trypan blue staining technique. The effect of adding FLV in the concentration corresponding to the EC₅₀ value of each cell line was examined. The results for all cells were 97% or higher over the control cells, and cytotoxicity by FLV was hardly recognized.

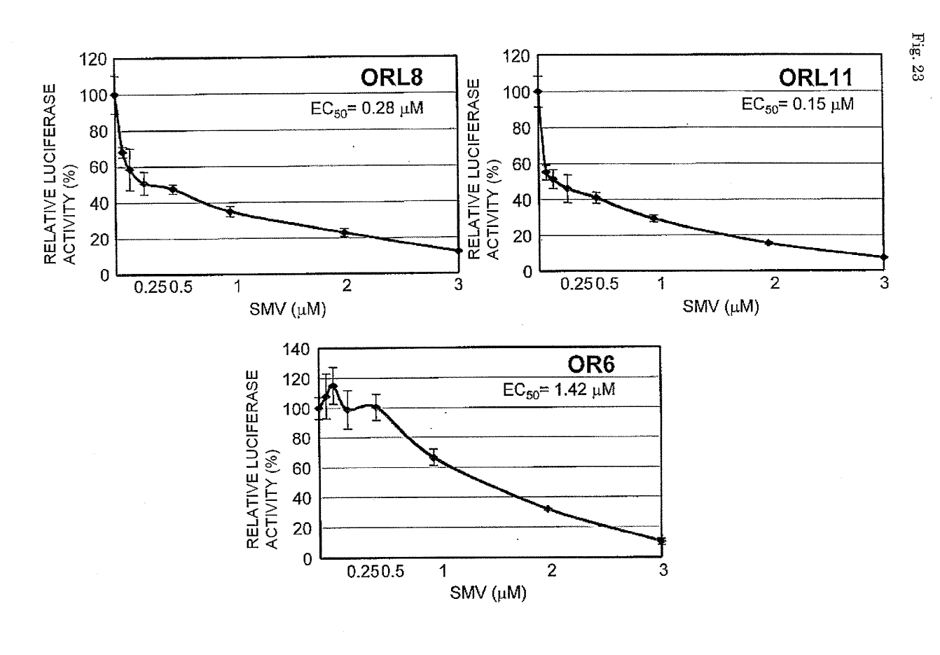

The same experiment was conducted using FLV obtained from a different manufacturer (LKT laboratories Inc., F4482, purity 99.5%), and EC₅₀ was calculated. The EC₅₀ values of ORL8, ORL11, and OR6 cells were 0.31 μM, 0.11 μM, and 1.44 μM, respectively (FIG. 22). By comparing these with the foregoing results, the values of the two results were about the same for ORL8 cells, whereas the current result had a higher effect for ORL11 cells, and the previous result had a slightly higher effect for OR6 cells. What is notable is the EC₅₀ value 0.11 μM in ORL11 cells, because it shows that the effect of FLV is maximized in this assay system. Reproducibility was confirmed by the EC₅₀ value of 0.14 μM obtained in a separate experiment (the results are not presented).

### (F) Pravastatin

Pravastatin (0, 0.25, 0.5, 1, 2, 3, 5, 10 μM) was added to each cell line, and luciferase activity after 72 hours was measured. There are previous reports that the anti-HCV effect by pravastatin was unconfirmable in a cell assay system using OR6 cells. As in OR6 cells, the anti-HCV effect by pravastatin was not confirmable in ORL8 and ORL11 cells in this experiment. In this respect, it can be said that there is no large difference between the HuH-7 cell line and the Li23 cell line (the results are not presented).

### (G) Simvastatin (SMV)

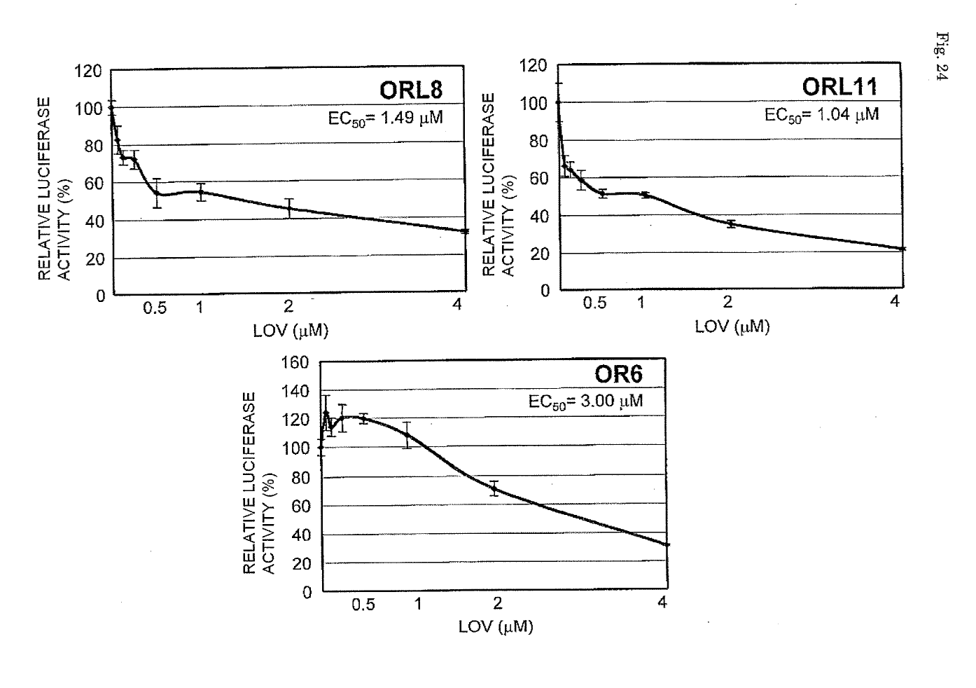

SMV (0, 0.063, 0.125, 0.25, 0.5, 1, 2, 3 μM: Wako chemical, 193-12051) was added to each cell line, and luciferase activity after 72 hours was measured. By analysis, the EC₅₀ values of ORL8, ORL11, and OR6 cells were 0.28 μM, 0.15 μM, and 1.42 μM, respectively. Representative results are presented in FIG. 23.

The anti-HCV effect of SMV is found with an assay using OR6 cells (see Ikeda et al., Hepatology 44: 117-125 (2006)). The ORL8 and ORL11 cells had EC₅₀ values (0.28 μM and 0.15 μM, respectively) considerably smaller than the EC₅₀ value (1.42 μM) of OR6 cells. This result suggests that SMV also has potential use in the treatment of patients with hepatitis C. What is notable is that SMV had the same level of activity as FLV in ORL8 cells, and that the anti-HCV activity of SMV was stronger than that of FLV in ORL11 cells. FLV topped SMV in anti-HCV activity in OR6 cells, but the order of anti-HCV activity was reversed in ORL11 cells. This result suggests the need for a comprehensive approach using ORL8 or ORL11 cells in addition to OR6 cells, and supports the usefulness of the cell assay systems that use ORL8 and ORL11 cells.

### (H) Lovastatin (LOV)

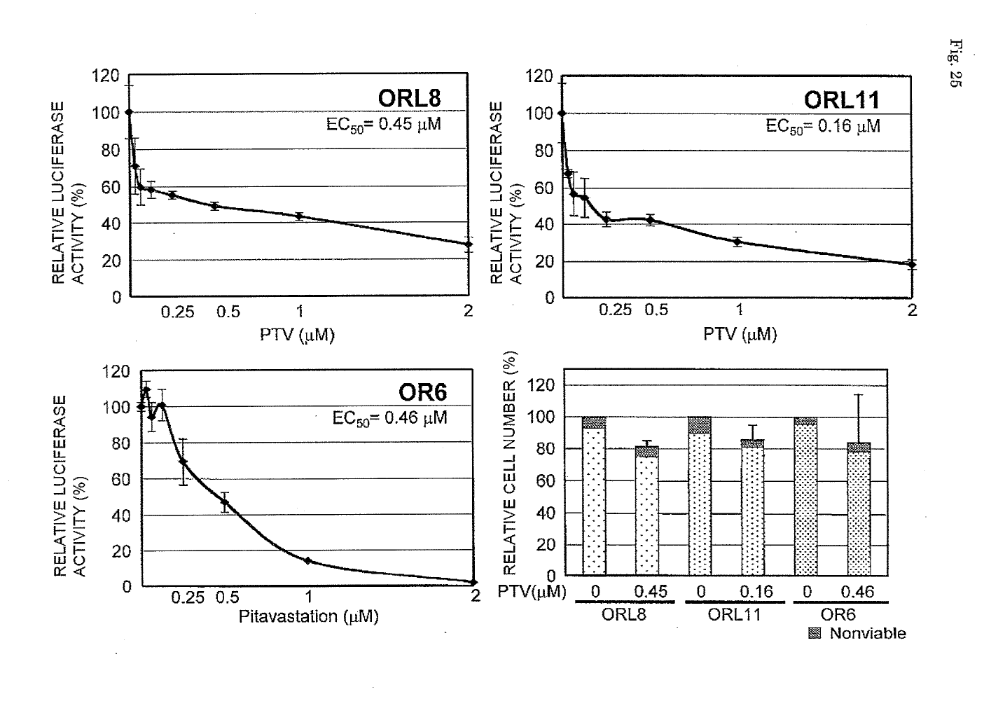

LOV (0, 0.063, 0.125, 0.25, 0.5, 1, 2, 4 μM: Wako chemical, 125-04581) was added to each cell line, and luciferase activity after 72 hours was measured. By analysis, the EC₅₀ values of ORL8, ORL11, and OR6 cells were calculated as 1.49 μM, 1.04 μM, and 3.0 μM, respectively. Representative results are presented in FIG. 24.

As also pointed out in other reports, LOV has high EC₅₀ values in replicon assays (meaning weak anti-HCV activities), and is not suited for clinical treatment. However, all of these reports use cell assay systems derived from HuH-7 cells. The results obtained in this experiment using the Li23 cell line-derived cell assay system suggest that it might be possible to find anti-HCV activities in drugs that are considered to show only weak anti-HCV activities, if any, in conventional cell assay systems.

### (I) Pitavastatin (PTV)

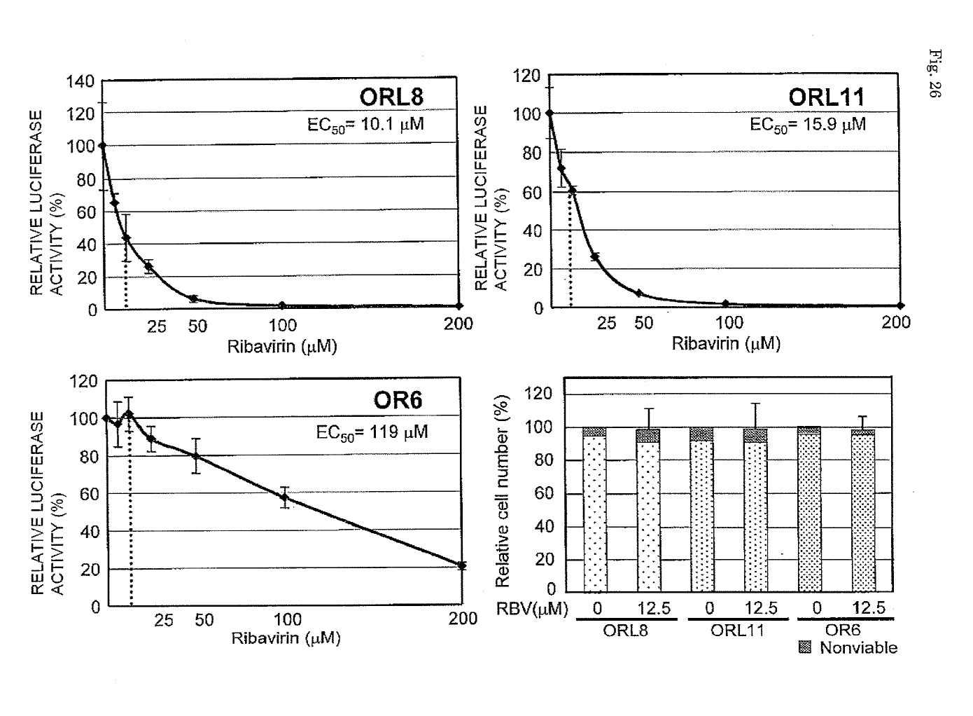

PTV (0, 0.032, 0.063, 0.125, 0.25, 0.5, 1, 2 μM: Tronto Research Inc., P531005 PTV lactone (prodrug)) was added to each cell line, and luciferase activity after 72 hours was measured. By analysis, the EC₅₀ values of ORL8, ORL11, and OR6 cells were calculated as 0.45 μM, 0.16 μM, and 0.46 μM, respectively. Representative results are presented in FIG. 25.

In order to ascertain that the decrease in luciferase activity in response to the addition of PTV was not due to the cell growth inhibition or cytotoxicity by PTV, the cells were cultured under the same conditions used for the luciferase assay, and counted using a trypan blue staining technique. The effect of adding PTV in the concentration corresponding to the EC₅₀ value of each cell line was examined. The results for all cells were 80% or higher over the control cells, and cytotoxicity by PTV was hardly recognized.

### (J) Ribavirin

Ribavirin (0, 3.13, 6.25, 12.5, 25, 50, 100, 200 μM: provided by Yamasa; purity>99.0%) was added to each cell line, and luciferase activity after 72 hours was measured. By analysis, the EC₅₀ values of ORL8, ORL11, and OR6 cells were calculated as 10.1 μM, 15.9 μM, and 119 μM, respectively. Representative results are presented in FIG. 26.

Ribavirin is currently used with PEG-IFN, and, despite side effects such as anemia, the drug has been used as a standard therapy because it provides better therapeutic effect than using PEG-IFN alone. However, what provides the anti-HCV effect of ribavirin remains elusive. In the measurement using an OR6 cell assay system prepared by the present inventors, ribavirin was shown to have anti-HCV effect with a high EC₅₀ value (76 μM) (Naka et al., BBRC, 330; 871-879, 2005). Broadly, four possibilities have been proposed concerning the anti-HCV effect of ribavirin:

(1) Ribavirin has RNA mutation inducing activity, and induces mutation in the HCV genome (causes error catastrophe at 100 μM or more);

(2) Inhibitory effect for the RNA-dependent RNA polymerase (NS5B) of HCV;

(3) Cell immunity enhancing effect, and effect by promotion of IFN-γ production; and

(4) Inhibitory effect for inosine-5′-monophosphate dehydrogenase (IMPDH).

An assay using ORL8 or ORL11 cells produced an unexpected result. Compared with the EC₅₀ (119 μM) obtained in the assay using OR6 cells, the assays using ORL8 and ORL11 cells yielded considerably lower EC₅₀ values of 10.1 μM and 15.9 μM, respectively.

In order to ascertain that the decrease in luciferase activity in response to the addition of ribavirin was not due to the cell growth inhibition or cytotoxicity by ribavirin, the cells were cultured under the same conditions used for the luciferase assay, and counted using a trypan blue staining technique. The effect of adding ribavirin in the concentration corresponding to the EC₅₀ value of each cell line was examined. The results for all cells were 98% or higher over the control cells, and cytotoxicity by ribavirin was hardly recognized. Cytotoxicity by ribavirin was not recognized at a concentration of 12.5 μM (the results are not presented).

The EC₅₀ value of 10 μM cannot be said as having a strong anti-HCV effect. However, the blood concentrations of patients undergoing ribavirin therapy are about 10 to 14 μM, and this range of concentration was found to be sufficient for enhancing the anti-HCV effect when PEG-IFN is used in combination. From the experiment results using the conventional HuH-7 cell line, it had been believed that HCV RNA replication could not be inhibited at the ribavirin blood concentration of a drug-administered patient. However, it was found from the foregoing results that ribavirin has activity as an HCV RNA replication inhibitor. It has been confirmed by Western blotting that, in ORL8 and ORL11 cells, the amounts of HCV proteins decrease correlatively with decrease in luciferase activity by ribavirin (data not presented). The results suggest that it might be possible to actually find anti-HCV activity in drugs that are considered to show only weak anti-HCV activities, if any, in conventional cell assay systems. It can therefore be said from the perspective of finding such anti-HCV agents that the present assay system can be a useful assay system.

### (K) Mizoribine

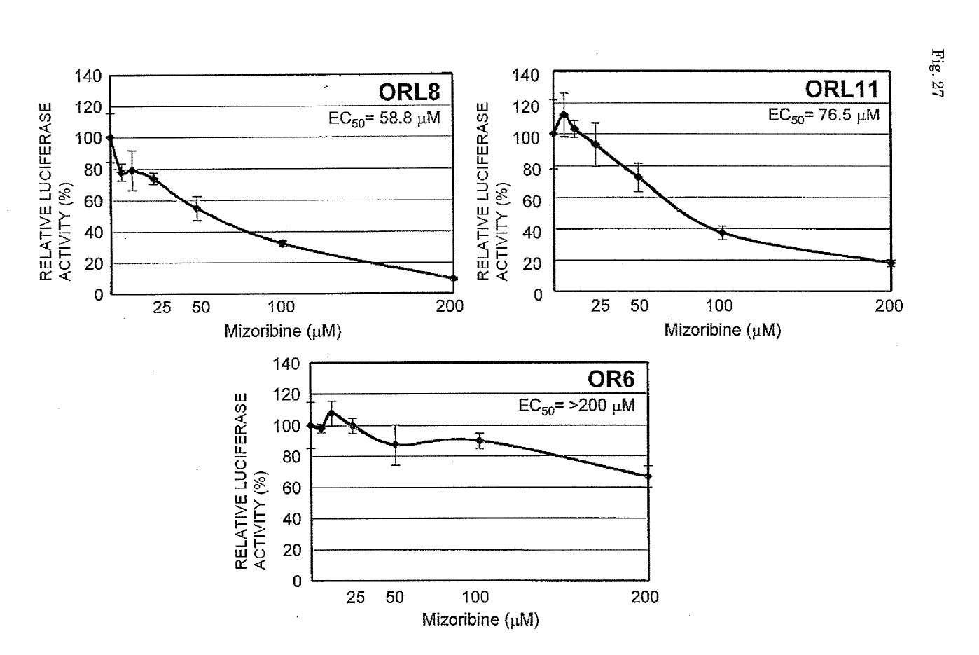

Mizoribine (0, 3.13, 6.25, 12.5, 25, 50, 100, 200 μM) was added to each cell line, and luciferase activity after 72 hours was measured. By analysis, the EC₅₀ values of ORL8 and ORL11 cells were calculated as 58.8 μM and 76.5 μM, respectively. In an assay system using OR6 cells, the value remained above 50% even at 200 μM. Representative results are presented in FIG. 27.

### (L) Geldanamycin

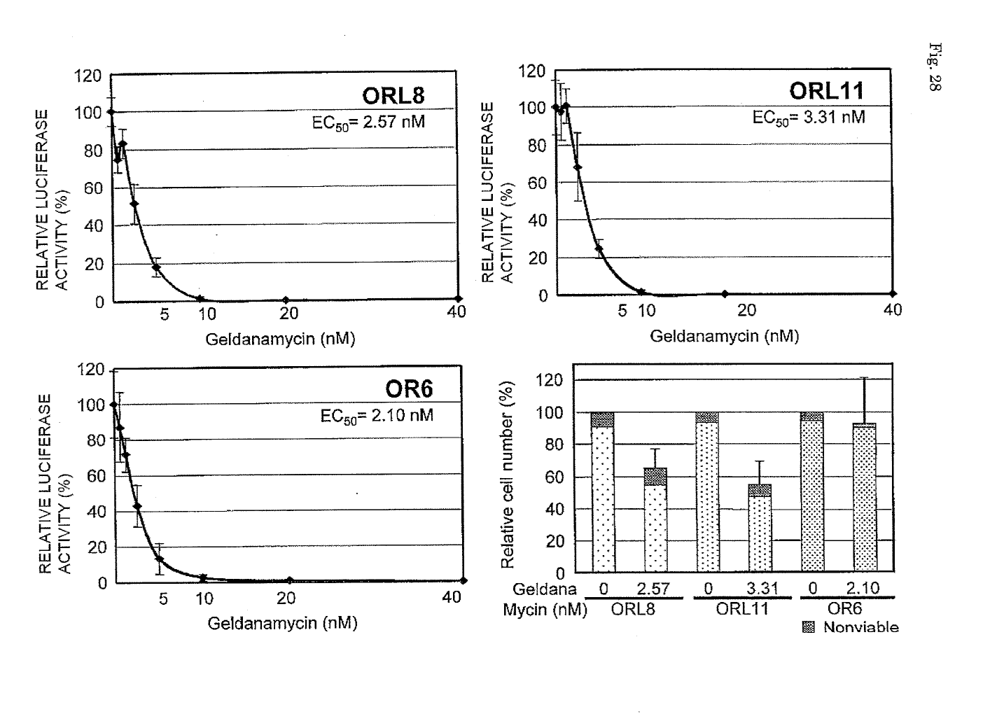

Geldanamycin (0, 0.63, 1.25, 2.5, 5, 10, 20, 40 nM: Wako chemical, 077-04571) was added to each cell line, and luciferase activity after 72 hours was measured. Geldanamycin (heat shock protein (Hsp90) inhibitor) is reported in a paper from other laboratory in which HCV replicon assay (using HuH-7 cell-derived cells) is used (Nakagawa et al., BBRC 353; 882-888, 2007), and the EC₅₀ value (analyzed 72 hours after the addition of the drug) has been calculated as 5.5 nM in Con1 strain, and 7.8 nM in N strain. Thus, in this experiment, assay was performed in such a manner that these values fell at the center of the drug concentration range. By analysis, the EC₅₀ values of ORL8, ORL11, and OR6 cells were calculated as 2.57 nM, 3.31 nM, and 2.10 nM, respectively. Representative results are presented in FIG. 28.

In this manner, the anti-HCV activity of geldanamycin was also confirmed in this assay system. The values obtained for the replication of full-length HCV RNA in HCV-O strain were lower than the reported values, but did not differ greatly among the different cells.

### (M) Myriocin

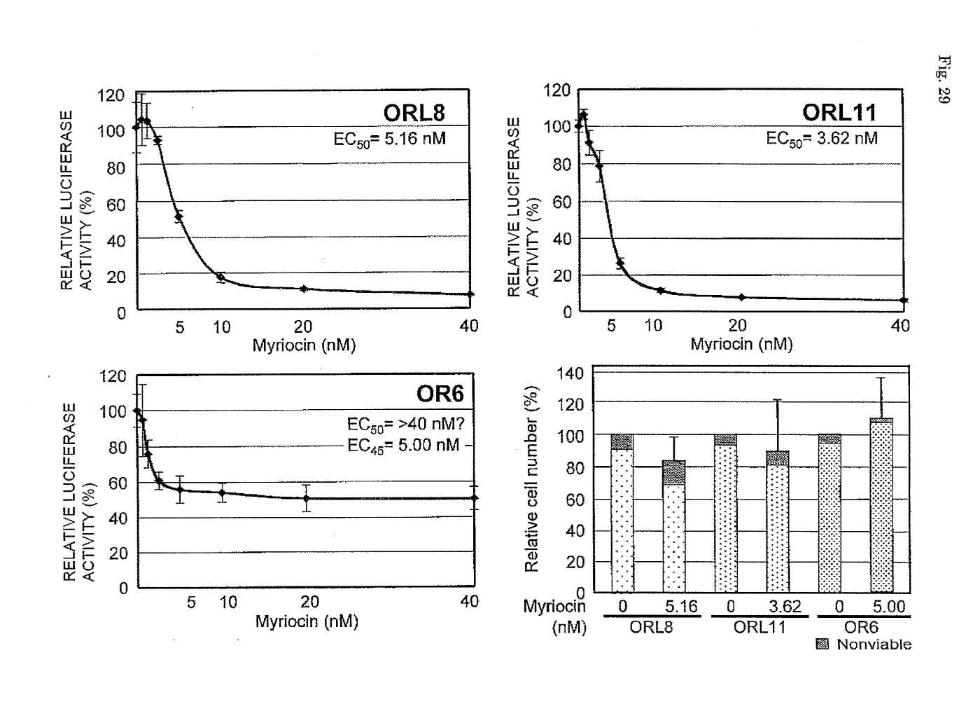

Myriocin (0, 0.63, 1.25, 2.5, 5, 10, 20, 40 nM: Sigma, M1177) was added to each cell line, and luciferase activity after 72 hours was measured. Myriocin (serine palmitoyl transferase inhibitor) is reported in a paper from other laboratory in which HCV replicon assay (using HuH-7 cell-derived cells) is used (Umehara et al., BBRC 346; 67-73, 2006), and the EC₅₀ value (analyzed 72 hours after the addition of the drug) has been calculated as 5.8 nM in Con1 strain. Thus, in this experiment, assay was performed in such a manner that these values fell at the center of the drug concentration range. By analysis, the EC₅₀ values of ORL8 and ORL11 cells were calculated as 5.16 nM and 3.62 nM, respectively. The results for these cells were almost the same as the values obtained in the Con-1 strain HCV replicon assay. However, in the assay using the HuH-7 cell-derived OR6 cells, activities slightly below 60% were maintained even at 5 nM to 40 nM, and, because the value did not fall below 50%, it was not possible to calculate EC₅₀ value. Although the reasons for these results remain unclear, it can be said that the ORL8 and ORL11 in the Li23 cell line that does not show the phenomenon observed in the cells of HuH-7 cell line provide cell assay systems suited for the evaluation of myriocin-related drugs. Representative results are presented in FIG. 29.

In order to ascertain that the decrease in luciferase activity in response to the addition of myriocin was not due to the cell growth inhibition or cytotoxicity by myriocin, the cells were cultured under the same conditions used for the luciferase assay, and counted using a trypan blue staining technique. The effect of adding myriocin in the concentration corresponding to the EC₅₀ value of each cell line was examined. The number of ORL8 cells was 83.5%, slightly below the result from the control cells. However, the results were 91% or higher in the other cells. It was considered from these results that the decrease in luciferase activity was due to the anti-HCV activity of myriocin.

### (N) Acetylsalicylic Acid (ASA)

There is a recent report describing the anti-HCV activity of ASA as revealed by experiments using the HCV replicons of Con1 strain (Trujillo-Murillo K, et al., Hepatology 47: 1462-1472 (2008)). Thus, the anti-HCV activity of ASA was investigated in the present assay system, and EC₅₀ was calculated.

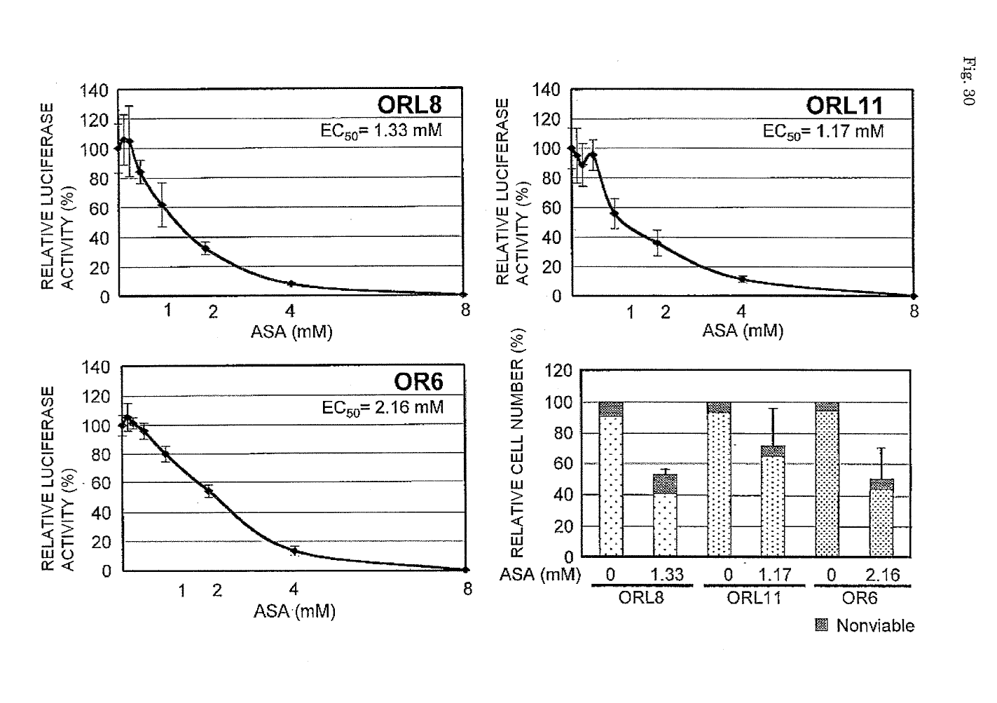

Myriocin (0, 0.125, 0.25, 0.5, 1, 2, 4, 8 mM) was added to each cell line, and luciferase activity after 72 hours was measured (the EC₅₀ value of ASA is reported to be 4 mM in the foregoing paper). By analysis, the EC₅₀ values of ORL8, ORL11, and OR6 cells were calculated as 1.33 mM, 1.17 mM, and 2.16 mM, respectively. These concentration values are slightly below the values obtained in the Con-1 strain HCV replicon assay, and apparently confirm the anti-HCV activity of ASA. However, because there was a clear decrease in cell count in cells treated at a concentration of 2 mM, the effect of the drug on cell growth was examined by treating the ORL8, ORL11, and OR6 cells with the foregoing concentrations of ASA. As a result, the cell count decreased to 53%, 72%, and 51% of the control cells. Representative results are presented in FIG. 30. The results suggest that the decrease in luciferase activity in at least ORL8 and OR6 cells are almost completely due to cell growth inhibition. The foregoing report does not give any consideration to the cell growth inhibitory effect of ASA, and it cannot be concluded that the ASA itself has anti-HCV activity. Thus, when presented with data showing a clear decrease in cell count in the drug concentration range used in the assay, it is necessary to count and compare the cells, and test whether the decrease in luciferase activity is due to a decrease in cell count.

### 5-6 Anti-HCV Effect by Combined Use of Drugs

The assay system using OR6 cells (see Patent Document 1) also can be used to measure the combined effect of drugs with IFN-α. Thus, assessment was made whether the present assay systems using the ORL8 cells and ORL11 cells were usable for the measurement of combined effect. Note that the concentrations of the drugs used are based on the EC₅₀ values above.

### (i) Combined Use of IFN-α and CsA

The results are presented in FIG. 31. The luciferase activities after the IFN-α treatment alone were 50% (ORL8), 41% (ORL11), and 67% (OR6). The values were 35% (ORL8), 48% (ORL11), and 73% (OR6) for CsA. The result for OR6 cells is slightly below the expected value (50%); however, this does not present itself as a problem for the measurement of combined effect. The luciferase activities after the combined use of the drugs decreased to 14% (ORL8), 13% (ORL11), and 33% (OR6), respectively. The values of the additive effect by the combined use expected from the results of single-agent treatment were 18% (ORL8), 20% (ORL11), and 49% (OR6). The actual values of the cell assay systems were lower than the expected values by 22% (ORL8), 35% (ORL11), and 33% (OR6), so the combined use of IFN-α and CsA was found to show some synergistic effect. The synergistic effect by the combined use of IFN-α and CsA (OR6 cell assay system) is described in Patent Document 1. Thus, the ORL8-cell and ORL11-cell assay systems also can be used to assay the combined effect of IFN-α and CsA as with the OR6-cell assay system.

### (ii) Combined Use of IFN-α and FLV

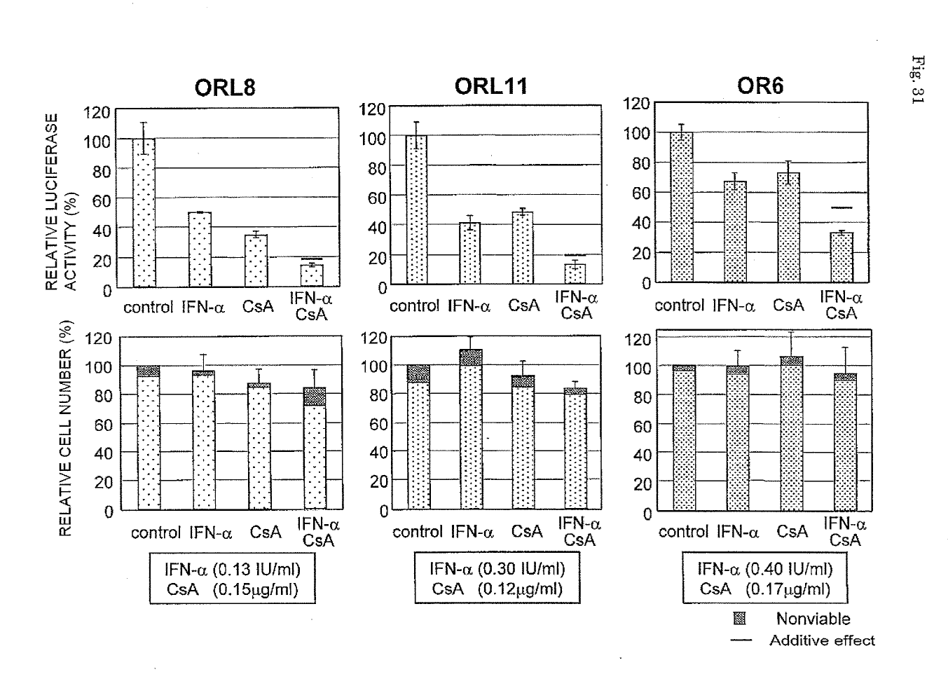

FLV (Calbiochem, 344095) was used. The results are presented in FIG. 32. The luciferase activities after the IFN-α treatment alone were 50% (ORL8), 41% (ORL11), and 67% (OR6). The values were 59% (ORL8), 51% (ORL11), and 55% (OR6) for FLV. The luciferase activities after the combined use of the drugs decreased to 29% (ORL8), 20% (ORL11), and 37% (OR6). The values of the additive effect by the combined use expected from the results of single-agent treatment were 30% (ORL8), 21% (ORL11), and 37% (OR6). These effects are considered additive because the results have good match with the actual values. However, upon checking the cell count, it was found that the drugs alone had the tendency not to inhibit almost any cell growth, but inhibit cell growth when used in combination.

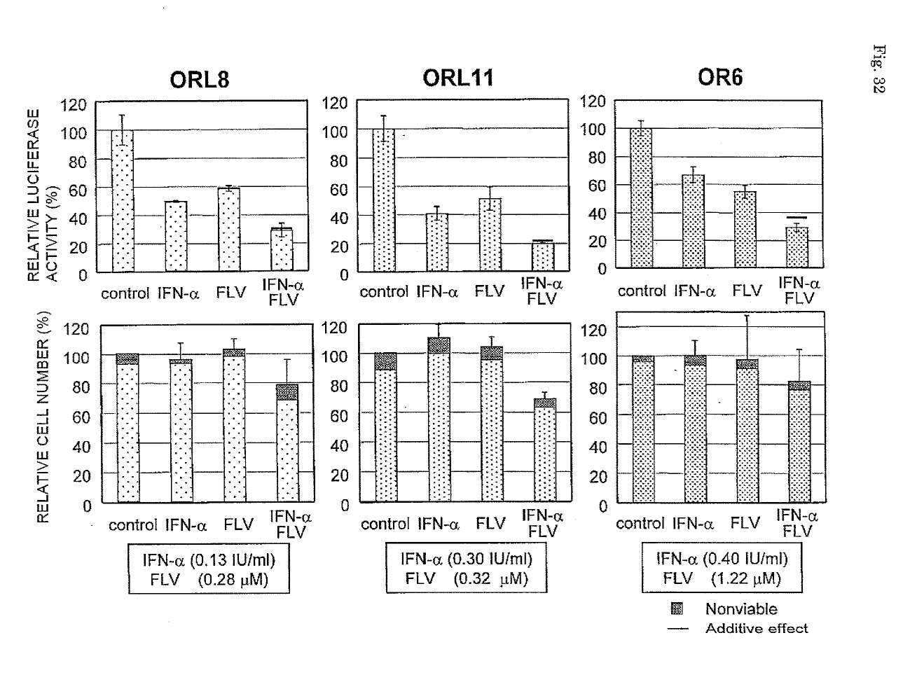

For the combined effect of IFN-α and FLV, assay was performed at different drug concentrations. FIG. 33 shows the results of assays using ORL8 cells and OR6 cells. IFN-α was added to make the final concentrations 0, 0.1, 0.2, and 0.4 IU/ml. FLV was added to make the final concentrations 0, 0.3, 0.6, and 1.2 μM. Luciferase activity was measured after 72 hours from the addition. The results were close to those expected from the results obtained from adding these agents alone. The combined effect of IFN-α and FLV represented by these results is considered additive, and the ORL8-cell assay system is considered to have better sensitivity than the OR6-cell assay system.

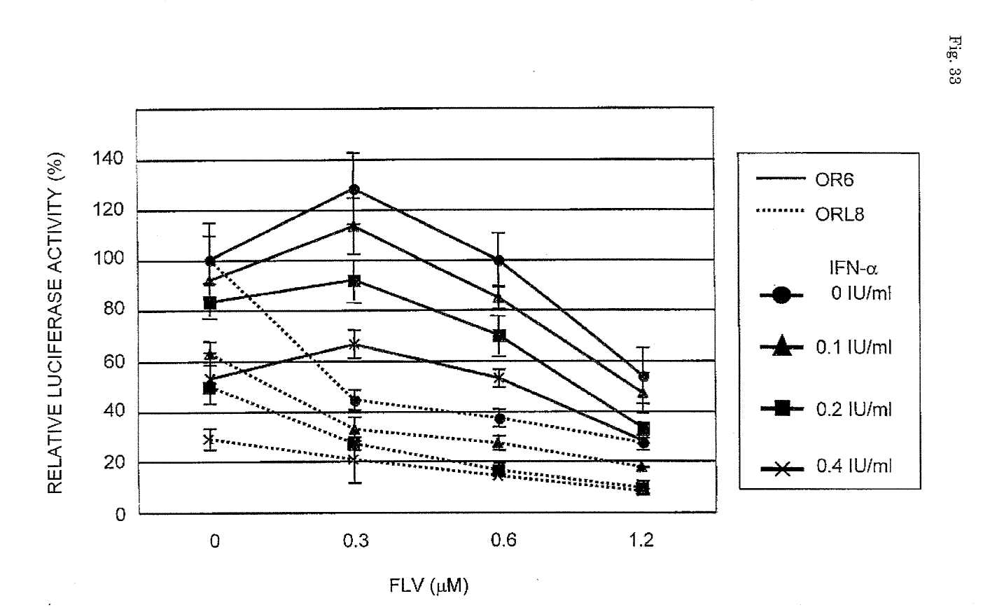

FIG. 34 shows the results of ORL11- and OR6-cell assays. The results were close to those expected from the results of single-agent treatment, and the luciferase activity had concentration-dependent attenuation patterns as did the ORL11 cells. It can also be considered from these results that the ORL11-cell assay system has better sensitivity than the OR6-cell assay system.

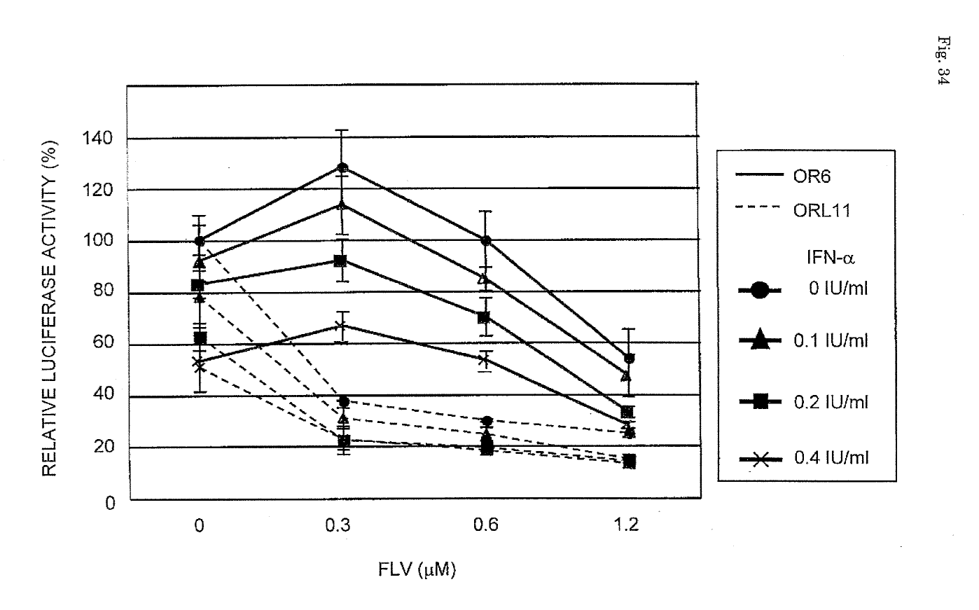

FIG. 35 shows the assay results from ORL8 and ORL11 cells. There is an overlap between the attenuation curves of luciferase activity between the two assay systems. The ORL8 cell line appeared to have slightly higher sensitivity to IFN-α; however, these assay systems were substantially the same in terms of sensitivity to the combined effect.

(iii) Combined Use of IFN-α and PTV

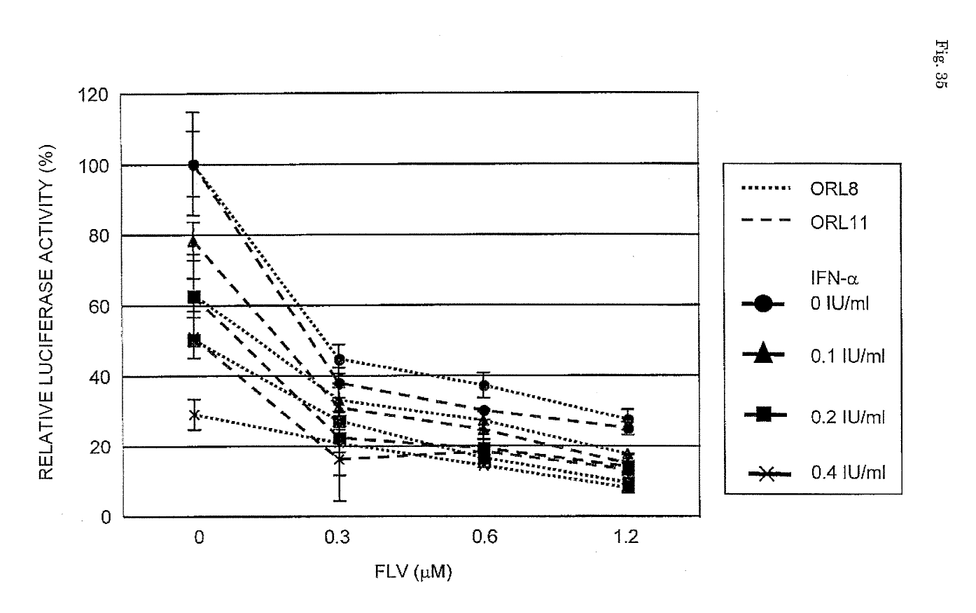

PTV (PTV lactone (prodrug)) was used. The results are presented in FIG. 36. The luciferase activities after the IFN-α treatment alone were 50% (ORL8), 41% (ORL11), and 67% (OR6). The values were 43% (ORL8), 49% (ORL11), and 56% (OR6) for PTV. The luciferase activities after the combined use of the drugs decreased to 24% (ORL8), 19% (ORL11), and 31% (OR6). The values of the additive effect by the combined use expected from the results of single-agent treatment were 22% (ORL8), 20% (ORL11), and 38% (OR6). The value of the OR6-cell assay system was lower than the expected value by little less than 20%. However, the effect was considered additive considering the results of the other cell assay systems together.

### (iv) Combined Use of IFN-α and Ribavirin

The results are presented in FIG. 37. It should be noted that the combined effect was examined under the fixed conditions of IFN-α at 0.25 IU/ml (the concentration expected to cause 35%, 55%, and 70% decreases in ORL8, ORL11, and OR6 cells, respectively), and ribavirin at 12.5 W (the concentration expected to cause 40% and 60% decreases in ORL8 and ORL11 cells, respectively, and no decrease in OR6 cells). The luciferase activities after the IFN-α treatment alone were 26% (ORL8), 48% (ORL11), and 75% (OR6). The values were 35% (ORL8), 49% (ORL11), and 97% (OR6) for ribavirin. The luciferase activity after the combined use of the drugs decreased to 11% (ORL8), 28% (ORL11), and 80% (OR6). The values of the additive effect by the combined use expected from the results of single-agent treatment were 9% (ORL8), 24% (ORL11), and 73% (OR6). The effect represented by these results was considered additive, even though the actual values in these cell assay systems were slightly higher (9 to 18%) than the expected values.

### 6: Infectious HCV Particle-Producing Cell Line

### 6-1 HCV Infection of OL Cured Cells

When HuH-7 cell-derived cells are used, only the HCV type 2a-derived JFH1 strain HCV can reproduce the HCV lifecycle. The ability of Li23 cell-derived cells to reproduce the HCV lifecycle was investigated. First, it was investigated whether the cured cells prepared from OL8 cells and OL11 cells obtained as the full-length HCV RNA replicating cells were capable of JFH1 strain HCV RNA replication.

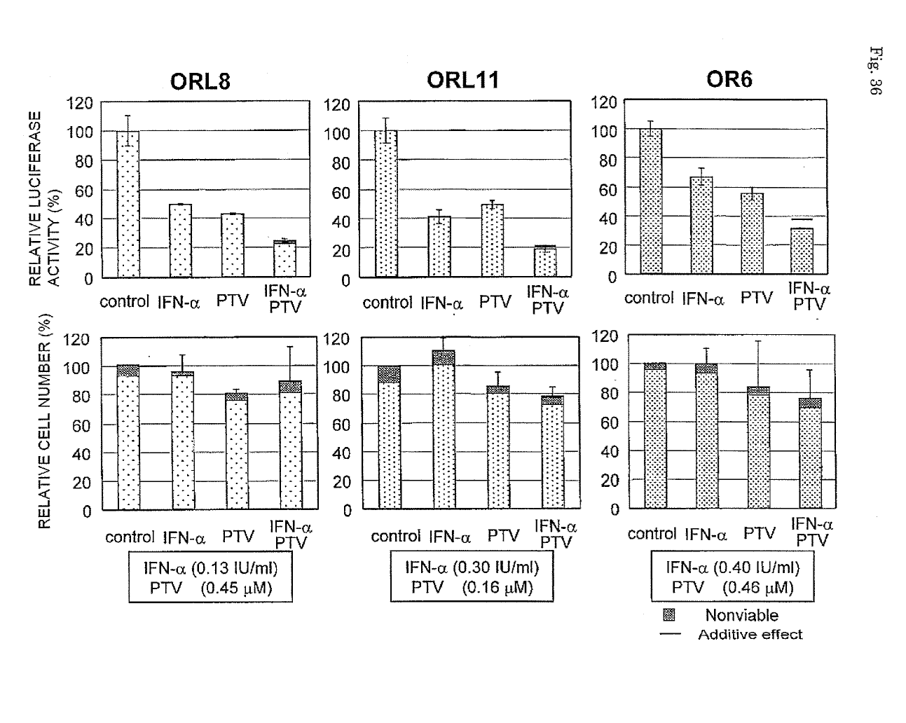

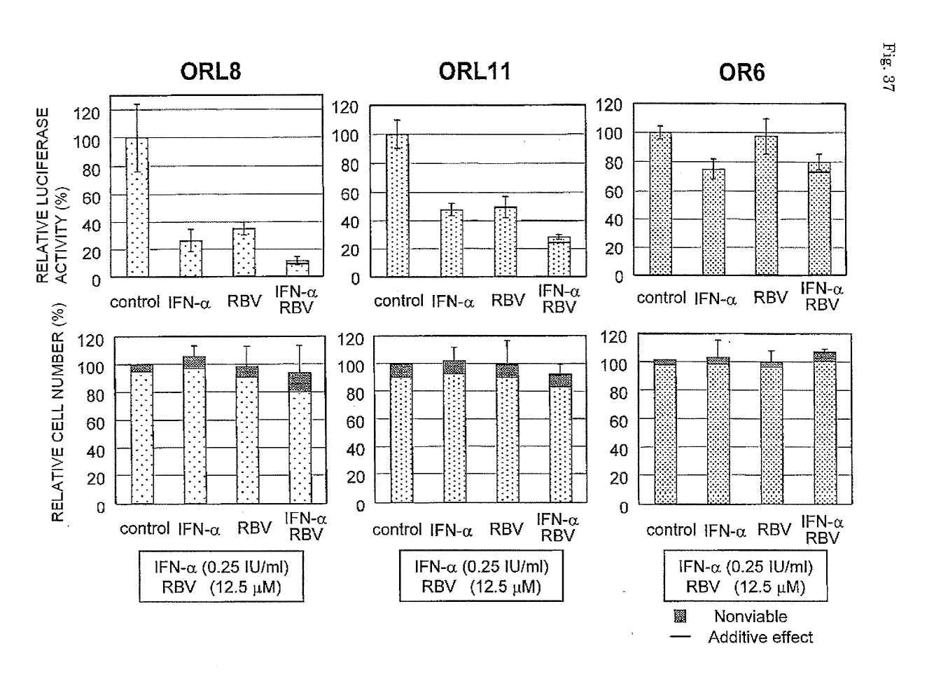

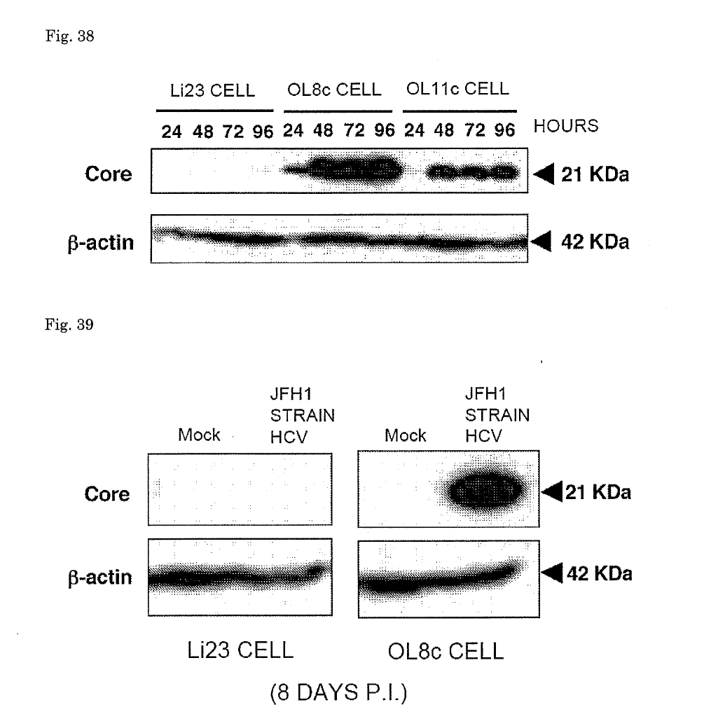

JFH1 strain HCV RNA (20 μg) was introduced into Li23 cells (parental strain), OL8c cells, and OL11c cells (2×10⁶ each), using the electroporation technique. Following the introduction, the cells were transferred to a 6-well plate (about 4×10⁵ each), and the expression levels of the core protein in each cell line ( 1/20 of the cells in each well was used for the analysis) were analyzed by Western blotting after 24, 48, 72, and 96 hours (FIG. 38). The core protein was not recognized at all in Li23 cells. In OL8c cells, the core protein was recognized 24 hours after the introduction, and enhancement of expression level was observed after 48 hours. The core protein was not detected in OL11c cells after 24 hours from the introduction; however, expression of the core protein was recognized after 48 hours. These results appear to be solely due to the replication and propagation of the JFH1 strain HCV RNA in the OL8c cells and OL11c cells, suggesting that the OL8c and OL11c cells are permissive for the replication of the HCV RNAs of not only the HCV-O strain but also the JFH1 strain.

RSc cells were infected with JFH1 strain HCV, and the supernatant after 145 days was used as virus fluid. The virus fluid was expected to contain 10^{5.3} TCID₅₀ of infectious HCV particles per ml. The RSc cells are HuH-7 cell-derived cloned cells, and efficiently and persistently produce infectious HCV particles with the JFH1 strain HCV RNA introduced into the cells (Ariumi et al., JVI 81: 13922-13926, 2007).

The virus fluid was added to Li23 cells (2×10⁴ cells/24 wells) and to OL8c cells (2×10⁴ cells/24 wells), and the medium was replaced after 2 hours. After 8 days from the infection, the expression level of core protein in each cell line was determined by Western blotting. Mock experiment was conducted in parallel using culture. An equivalent of 2×10⁴ cells was used for the assay. While the core protein was not detected at all in Li23 cells, strong core protein expression was recognized in OL8c cells (FIG. 39). The results suggest that the OL8c cells are permissive not only for the replication of JFH1 strain HCV RNA but also for the infection of JFH1 strain HCV.

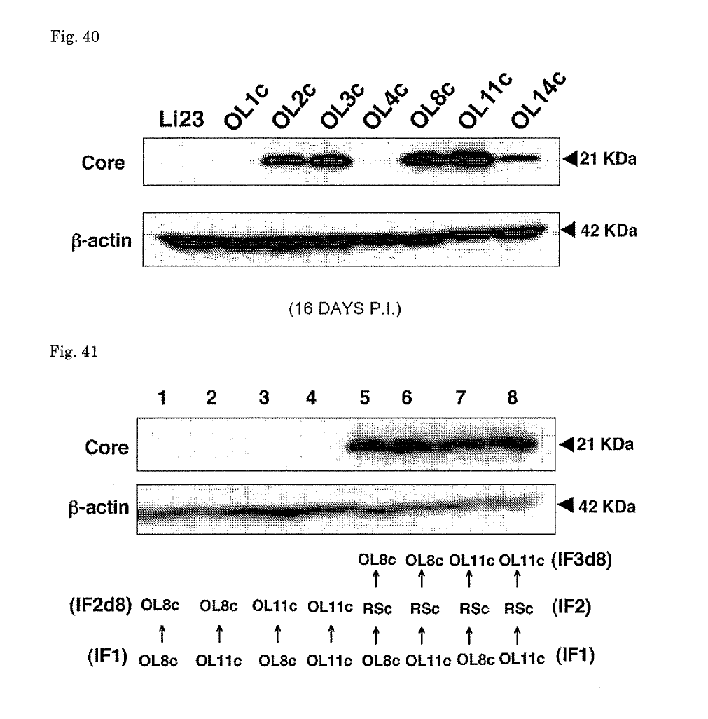

Non-OL8c cured cells were also examined with respect to JFH1 strain HCV infection and propagation. JFH1 strain HCV infection experiment was conducted using Li23, and cured cells (OL1c cells, OL2c cells, OL3c cells, OL4c cells, OL8c cells, OL11c cells, and OL14c cells) prepared from various cloned OL cells. The expression level of core protein in each cell line after 16 days from the infection was determined by Western blotting. While the core protein was not detected at all in Li23 cells, strong core protein expression was recognized in OL8c cells even after 16 days from infection (FIG. 40). This suggests sustained HCV RNA replication in the cells after the infection. The OL2c cells, OL3c cells, and OL11c cells had the same level of core protein expression as OL8c cells, whereas expression was weak in OL14c cells. The core protein was not detected at all in OL1c cells and OL4c cells. Given the lack of correlation with the amount of full-length HCV RNA (the replication level of HCV-O strain HCV RNA) in the cells, the differences among the cell clones may be due to differences in permissiveness in the HCV infection step, or differences in replication efficiency based on different HCV strains.

### 6-2 Production of Infectious HCV Particles from OL Cured Cells

Production of infectious HCV particles from JFH1 strain HCV-infected OL8c and OL11c cells was examined. Note that the cells were infected by replacing medium after 2 hours from the addition of virus fluid to the cells (FIG. 41).

The culture supernatants of OL8c and OL11c cells (2×10⁴ cells/24 wells each) 7 days post infection (100 μl each, IF1 in the figure) were used to infect OL8c and OL11c cells (2×10⁴ cells/24 wells each). The core protein expression level of each cell line 8 days post infection (IF2d8 in the figure) was analyzed by Western blotting. The results are shown in lanes 1 to 4. The core protein was not detected in OL8c and OL11c cells, and it was not possible to confirm production of infectious HCV particles from OL8c and OL11c cells.

The culture supernatants of OL8c and OL11c cells (2×10⁴ cells/24 wells each) 7 days post infection (100 μl each, IF1 in the figure) were used to infect RSc cells (2×10⁴ cells/24 wells). The culture supernatant 7 days post infection (100 μl IF2 in the figure) was used to infect separately prepared OL8c and OL11c cells (2×10⁴ cells/24 wells each). The core protein expression level in each cell line 8 days post infection (IF3d8 in the figure) was analyzed by Western blotting. The results are shown in lanes 5 to 8. The core protein was detected in OL8c and OL11c cells. The result suggests small production of infectious HCV particles from OL8c and OL11c cells, and production and amplification of infectious HCV particles via RSc cells.

Because small production of infectious HCV particles from OL8c and OL11c cells was indicated, the HCV infected cells were continuously cultured for 27 days, and analyzed by Western blotting.

The culture supernatants of OL8c and OL11c cells (2×10⁴ cells/24 wells each) 7 days post infection (100 μl each, IF1 in the figure) were used to infect OL8c and OL11c cells (2×10⁴ cells/24 wells each). As a control of HCV-producing cells, Mock experiment using a Li23 cell supernatant or culture was also conducted (FIG. 42). The core protein expression level in each cell line 27 days post infection (IF2d27 in the figure) was analyzed by Western blotting. It was found that the expression level of core protein in the cells was very high in the infection of RSc cells with the supernatants of the JFH1 strain HCV-infected OL8c and OL11c cells. Conceivably, this is due to the propagation of infectious HCV particles via RSc cells.

The core protein 27 days post infection reached the detectable level by Western blot analysis only when the culture supernatant derived from the JFH1 strain HCV-infected ORL8c cells were used to reinfect OL8c cells. This fact suggests the completion of the HCV lifecycle in OL8c cells, specifically, reproduction of JFH1 strain HCV as infectious particles after replication and propagation in the infected OL8c cells, and replication and propagation of the infectious particles in the reinfected OL8c cells.

### 6-3 Preparation of ORL Cured Cell Lines

Though OL8c cells were shown to be capable of reproducing the HCV lifecycle, the capability is far below those of other cells such as RSc cells. Further, from the practical standpoint, experiment takes time. Thus, ORL8c and ORL11c cells considered to be superior to OL8c and OL11c cells in terms of HCV RNA replication environment were prepared by adding IFN-γ (10³IU/μl) to ORL8 and ORL11 cells in the absence of G418.

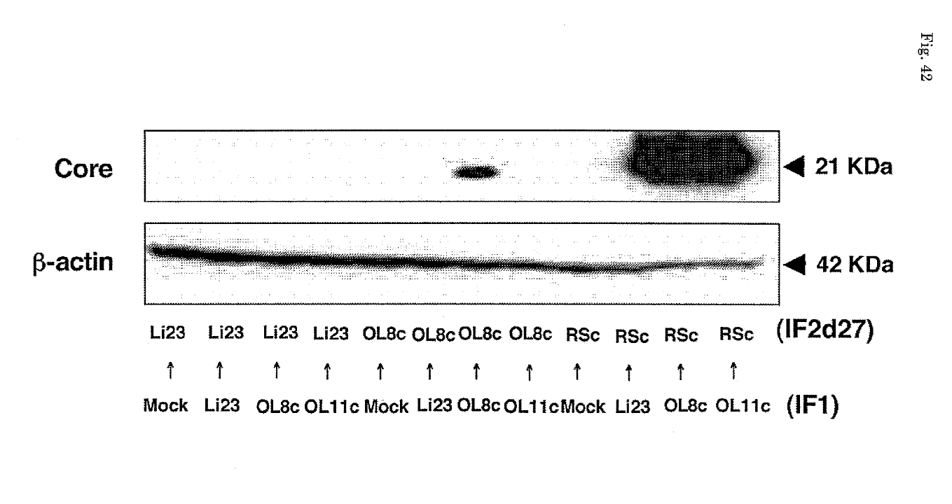

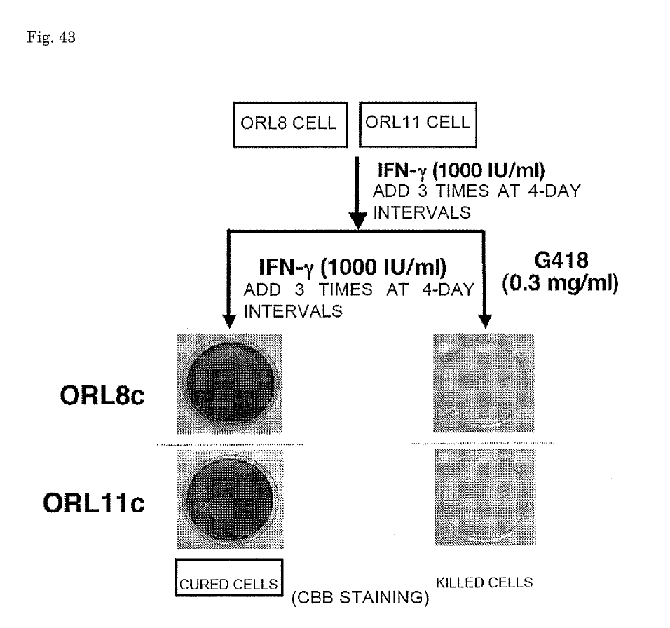

About 5×10⁵ ORL8 cells and ORL11 cells were inoculated on dishes having an outer diameter of 10 cm, and IFN-γ (1,000 IU/ml) was added three times at 4-day intervals. The cells were appropriately subcultured when the dish became full. The cells subcultured after the 3rd addition of IFN-γ were divided into two groups of dishes. One group contained medium supplemented with G418 (0.3 mg/ml) and NaHCO₃ (0.15%). The petri dish in the other group was continuously used to culture the cells with the medium alone. IFN-γ was then added three times to each group while the cells were subcultured as required, followed by CBB staining. While the cells cultured in the G418-free medium grew and filled the dish (cured cells), the cells were completely killed when continuously cultured in the medium supplemented with G418 (FIG. 43).

### 6-4 Production of Infectious HCV Particles from ORL Cured Cells

The prepared cured cells (ORL8c or ORL11c cells) were used for JFH1 strain HCV infection experiment, and HCV particle production capability was examined. As noted above, the Rsc cells used as control are HuH-7 cell-derived cloned cells, and efficiently and persistently produce infectious HCV particles with the JFH1 strain HCV RNA introduced into the cells (Ariumi et al., JVI 81: 13922-13926, 2007).

The culture supernatants of RSc cells, ORL8c cells, and ORL11c cells (2×10⁴ cells each (3×10⁴ cells for ORL11c cells)/24 wells; 100 μl each) 7 days post infection were used to infect separately prepared RSc cells (2×10⁴ cells/24 wells). Mock infection experiment was also conducted using culture. The core protein expression levels in each cell line 7 days and 14 days post infection (IF2d7 and IF2d14, respectively, in the figure) were analyzed by Western blotting (lanes 1 to 4 in FIG. 44). The core protein expression levels were substantially the same in lanes 2, 3, and 4. That is, the JFH1 strain HCV-infected RSc, ORL8c, and ORL11c cells produced substantially the same level of infectious HCV, and the ORL8c and ORL11c cells were shown to have higher infectious HCV production capability than OL8c and OL11c cells, comparable to that of HuH-7 cell-derived RSc cells.

The culture supernatants of RSc and ORL8c cells (2×10⁴ cells/24 wells each) 7 days post infection (100 μl each) were used to infect separately prepared ORL8c cells (2×10⁴ cells/24 wells). Mock infection experiment was also conducted using culture. The core protein expression levels in each of the cells 7 days, 14 days, 21 days, and 30 days post infection (IF2d7, IF2d14, IF2d21, and IF2d30, respectively, in the figure) were analyzed by Western blotting (lanes 5 to 7 in FIG. 44). The level of the core protein produced in the ORL8c cells infected with HCV produced from RSc cells became maximum after 7 days from infection, remained at almost the same level until day 21, and decreased by day 30 (lane 6). On the other hand, the core protein produced in the ORL8c cells infected with HCV produced from ORL8c cells had a substantial expression level by 7 days post infection, and the expression level increased time-dependently, and was maintained at higher levels even after 30 days relative to day 7 (lane 7). These results suggest that the HCV produced in ORL8c cells reinfects ORL8c cells, and that HCV production is maintained for at least 1 month. ORL8c cells were found to have infectious HCV particle production capability far superior to that of OL8c cells, which is the parental cell line of ORL8c cells, and such HCV production capability was comparable to that of HuH-7 cell-derived RSc cells.

The culture supernatants of RSc and ORL11c cells (2×10⁴ cells or 3×10⁴ cells/24 wells each) 7 days post infection (100 μl each) were used to infect separately prepared ORL11c cells (3×10⁴ cells/24 wells). Mock experiment was also conducted using culture. The core protein expression levels in each cell line 7 days, 14 days, 21 days, and 30 days post infection (IF2d7, IF2d14, IF2d21, and IF2d30, respectively, in the figure) were analyzed by Western blotting (lanes 8 to 10 in FIG. 44). As in the case of ORL8c cells, the level of the core protein produced in the ORL11c cells infected with HCV produced from RSc cells became maximum after 7 days from infection, remained at about the same level until day 21, and decreased by day 30 (lane 9). On the other hand, the core protein was not produced in the ORL11c cells infected with HCV produced from ORL11c cells (lane 10). It was found from these results that, unlike ORL8c cells, the ORL11c cells were permissive for infection and propagation of HCV produced from RSc cells, but not permissive for reinfection of ORL11c cells by the ORL11c cell-produced HCV and for propagation of the ORL11c cell-produced HCV.

### 6-5 HCV RNA Replication Level in ORL Cured Cells

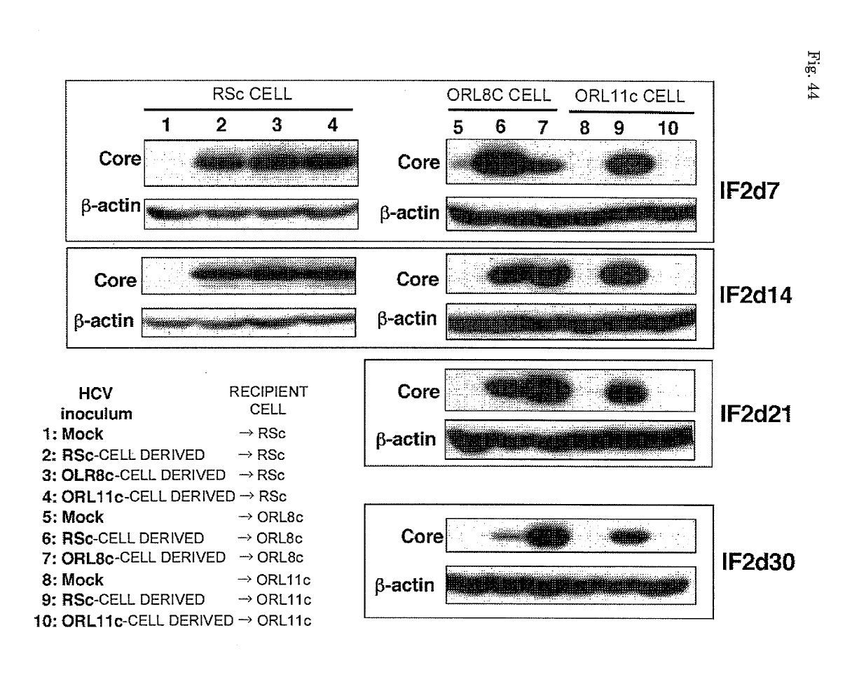

For detection of double-stranded RNA (dsRNA), a replication intermediate of HCV RNA, in JFH1 strain HCV-infected ORL8c cells (IF2d7), ORL8c cells were observed using the immunofluorescent technique with anti-dsRNA antibodies, according to the foregoing procedure. JFH1 strain HCV-infected RSc cells were used as positive control, and mock-infected ORL8c cells as negative control. The results of observation using a confocal laser scanning microscope are shown in FIG. 45 (bar length, 20 μm).

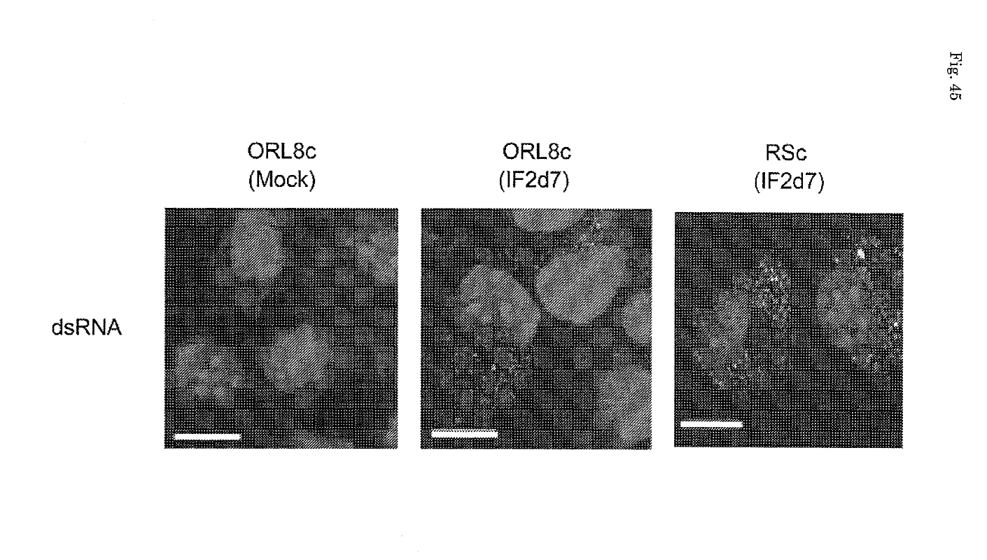

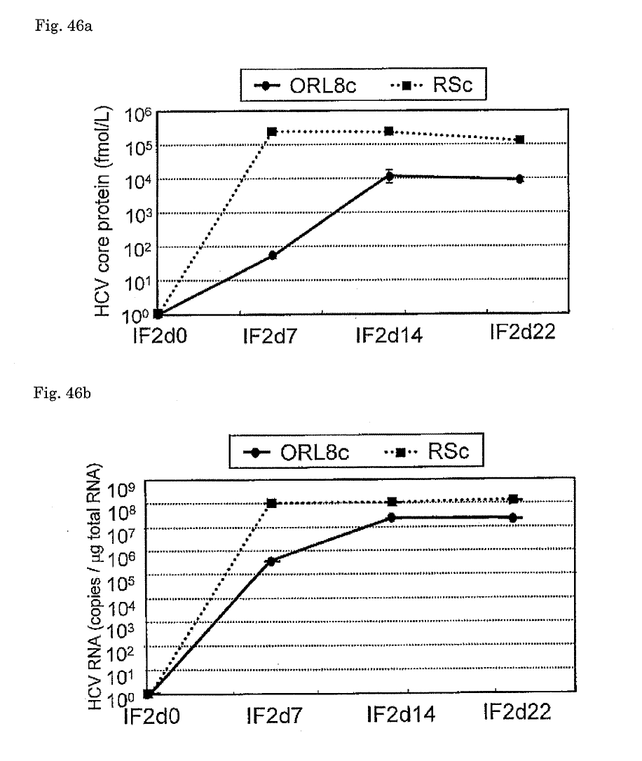

As shown in the figure, dot-like fluorescence scattered over the cytoplasm was observed in HCV-infected ORL8c and RSc cells. Such fluorescence was not observed at all in the mock-infected ORL8c cells. These results can be taken as the basis for the specific detection of dsRNA (replication intermediate of HCV RNA) in ORL8c cells. Note that the fact that the fluorescence intensity observed in ORL8c cells was comparable to that of RSc cells suggests that the HCV RNA replication level in ORL8c cells does not differ greatly from that in RSc cells, as shown in FIG. 46(b).

The HCV replication levels in ORL8c cells and RSc cells were compared. In order to detect and quantify the HCV particles released into the culture supernatants of ORL8c cells and RSc cells at the same time points used in FIG. 44 (7 days, 14 days, 21 days, and 30 days post infection; IF2d7, IF2d14, IF2d21, and IF2d30, respectively, in the figure), the secretion levels of HCV core protein in the culture supernatants of JFH1 strain HCV-infected ORL8c and RSc cells (IF2d7, IF2d14, IF2d22) were measured by ELISA (measurement was made by Mitsubishi BCL). Experiment was conducted three times, and 1 ml of supernatant was used for the measurement (FIG. 46(a)).

As shown in the figure, in RSc cells, the level of HCV particles released into the culture supernatant reached 10⁵ fmol/L or more 7 days post injection, and leveled off or decreased thereafter. On the other hand, in ORL8c cells, the HCV particle level remained low at 10² fmol/L 7 days post infection, increased to 10⁴ fmol/L by 14 days post infection, and remained at the same level until day 22 post infection. Taken together, it was found that the HCV particle production capability of ORL8c cells was one order of magnitude smaller than that of RSc cells. However, this level was found to be sufficient for the behavior analysis of various viruses.

The HCV RNAs in these cells were quantified according to the procedure of real-time LightCycler PCR described above. Experiment was conducted three times (FIG. 46(b)). In RSc cells, the RNA level reached 10⁸ copies/μg total RNA at day 7 post infection, and remained at almost the same level until day 22 post infection, as with the released level of core protein into the culture supernatant. On the other hand, in ORL8c cells, the RNA level was only about 5×10⁵ copies/μg total RNA at day 7 post infection. However, the RNA level reached about 5×10⁷ copies/μg total RNA on day 14 post injection, and remained at the same level until day 22. It was found from these results that high HCV RNA replication levels were maintained in the both cells.

### 6-6 The Correlation between HCV Receptor Expression Level and HCV Particle Production Capability

For comparison of HCV receptor expression levels in various cells, the expression level of mRNA was measured using a RT-PCR method and a quantitative RT-PCR method.

HuH-7, RSc, Li23, ORL8c, and ORL11c cells were cultured in 10-cm plates (medium, 10 ml). Total RNA was extracted from these cells using an RNeasy Mini Kit (Qiagen) according to the manufacturer's experiment protocol attached to the kit. Using 2 μg of RNA as a template, reverse transcription (RT) reaction was performed with SuperScript™ II reverse transcriptase (Invitrogen) and oligo dT (Invitrogen) according to the manufacturer's experiment protocols. The resulting cDNA was used as template, and PCR was performed with the primer sets shown in Table 3. PCR amplification products were detected by ethidium bromide staining after 3% agarose gel electrophoresis.

TABLE 3
Primers used for RT-PCR analysis

| Gene | | | | Amplification | Number of |
|---|---|---|---|---|---|
| (Accession No.) | Direction | Base sequence | | product (bp) | cycles |
| CD81 | Forward | ACCTTCCAC | SEQ ID NO: 18 | 222 | 25 |
| (NM_004356) | | GAGACGCTT | | | |
| | Reverse | CAGGATCATCT | SEQ ID NO: 19 | | |
| | | CGAAGATCATG | | | |
| SR-B1 | Forward | GGTGCGGCG | SEQ ID NO: 20 | 225 | 25 |
| (NM_005505) | | GTGATGATG | | | |
| | Reverse | CCCAGAGTCGG | SEQ ID NO: 21 | | |
| | | AGTTGTTGAG | | | |
| CLDN1 | Forward | GGGGTGCGATA | SEQ ID NO: 22 | 129 | 25 |
| (NM_021101) | | TTTCTTCTTG | | | |
| | Reverse | GAGCCTGACCA | SEQ ID NO: 23 | | |
| | | AATTCGTACC | | | |
| OCLN | Forward | TTCACTTCTA | SEQ ID NO: 24 | 235 | 25 |
| (NM_002538) | | CAAATGGACC | | | |
| | Reverse | TAGCCTCCGT | SEQ ID NO: 25 | | |
| | | AGCCATAGCC | | | |
| GAPDH | Forward | GACTCATGACC | SEQ ID NO: 26 | 334 | 22 |
| (NM_002046) | | ACAGTCCATGC | | | |
| | Reverse | GAGGAGACCAC | SEQ ID NO: 27 | | |
| | | CTGGTGCTCAG | | | |

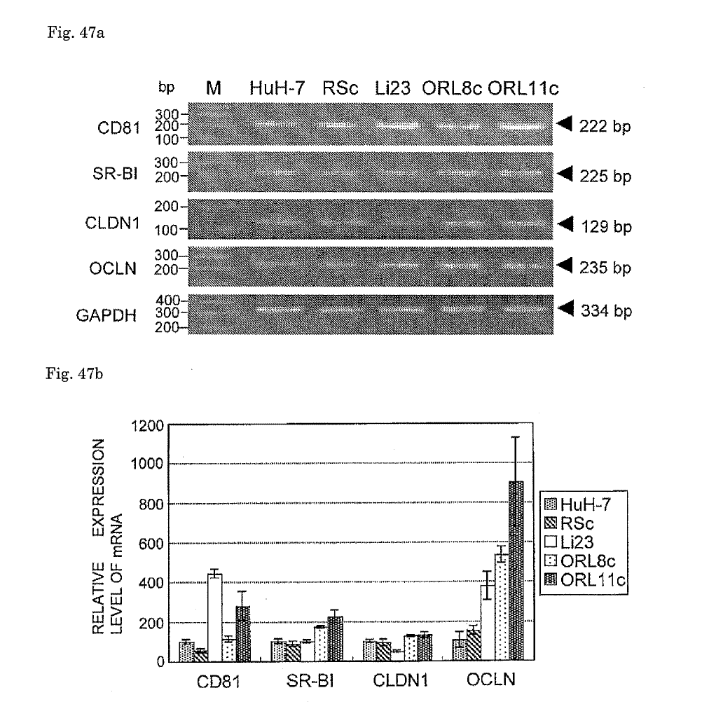

Expression of CD81, SR-B1, CLDN1 (Claudin-1), and OCLN (Occludin), reported to be HCV receptors, was confirmed in all cells along with GAPDH as internal control, though the detected bands had slightly different shades (FIG. 47(a)). For details of these HCV receptors, see Burlone M. E. and Budkowska A. Hepatitis C virus cell entry: role of lipoproteins and cellular receptor, Journal of General Virology 90, 1055-1070 (2009).

The cDNA was also used for quantification of HCV RNA according to the procedure of real-time LightCycler PCR. As a result, HCV receptor expression was confirmed in all cells as in the result of qualitative experiment presented in FIG. 47(a), and there was no HCV receptor that had a lower expression level only in ORL8c cells relative to RSc cells. Further, CLDN1 and OCLN had higher expression levels in ORL8c cells than in RSc cells (FIG. 47(b)).

### 6-7 Establishment of Full-Length HCV RNA Replicating Cell Line Derived from Non-HCV-O HCV Strains

ORL8c cells are cured cells produced by IFN-γ treatment that excludes HCV RNA from cells capable of replicating luciferase gene-carrying full-length HCV RNA derived from HCV-O strain. Thus, with ORL8c cells, it might be possible to establish cell lines capable of replicating full-length HCV RNA that derives from non-HCV-O HCV strains.

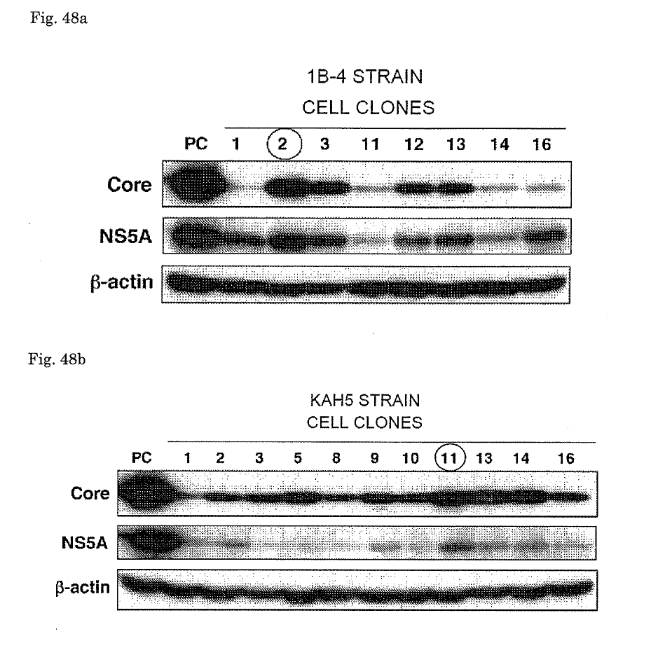

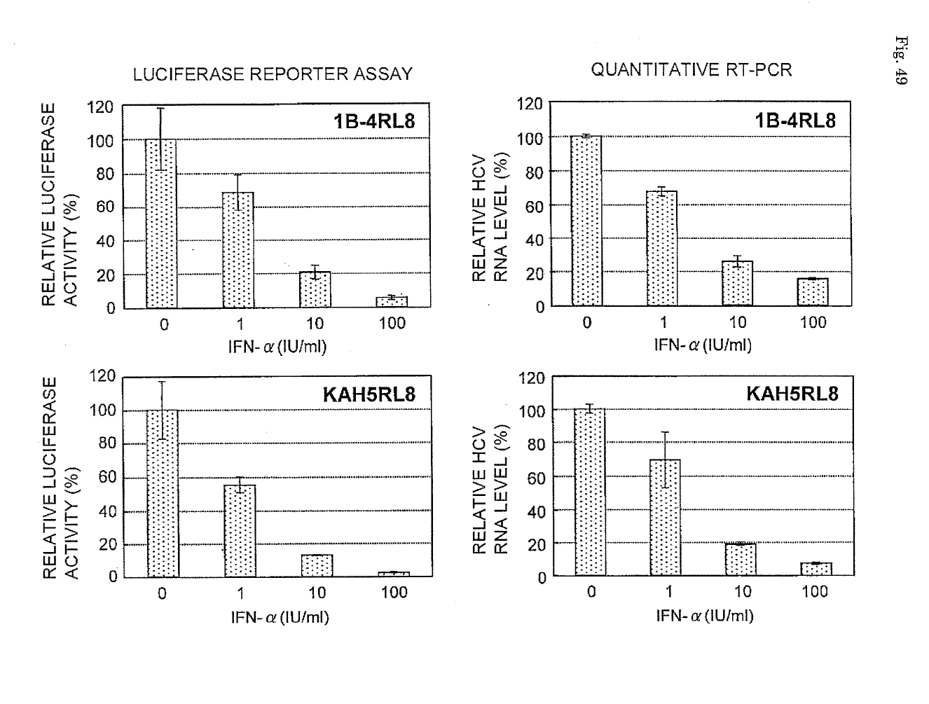

Luciferase gene-carrying full-length HCV RNA (10 μg) synthesized in vitro using the template plasmids (p1B-4RN/C-5B and pKAH5RN/C-5B) prepared from the HCV carrier strain 1B-4 (genotype 1b) and the acute hepatitis patient-derived HCV strain KAH5 (genotype 1b), respectively, was introduced into 2×10⁶ ORL8c cells using the electroporation technique. The plasmid p1B-4RN/C-5B had mutations Q1067R and S2200R introduced at two locations. The plasmid pKAH5RN/C-5B had mutations Ins2040K (insertion of lysine at position 2040 of the HCV polyprotein), R2328Q, E2401G, and V2416A introduced at four locations. S2200R and Ins2040K are known adaptive mutations. Starting from day 2 after the introduction of RNA, the medium was replaced with G418 (0.3 mg/ml)-containing medium every 4 days, and the cells were cultured for about 3 weeks. Cells believed to have high levels of sustained replication of luciferase gene-carrying full-length HCV RNA were obtained as G418-resistant colonies (clones). For the selection of cell clones having high HCV RNA replication levels from these clones, the expression levels of core protein and NS5A protein were analyzed by Western blotting (FIG. 48). OR6c cell-derived cells that showed efficient replication of HCV-O strain-derived full-length HCV RNA (with four adaptive mutations) were used as positive control (PC in the figure).

As shown in FIG. 48(a), the 1B-4 strain had the highest level of HCV protein expression in clone #2. Cell clone #2 was grown into a new cell line (will be referred to as “1E-4RL8 cells”). As shown in FIG. 48(b), the KAH5 strain had the highest level of HCV protein expression in clone #11. Cell clone #11 was grown into a new cell line (will be referred to as “KAH5RL8 cells”).

The 1B-4RL8 and KAH5RL8 cells were examined with regard to a possible correlation between luciferase activity and HCV RNA level (FIG. 49). The graphs on the left represent the measured luciferase activities 24 hours after the addition of IFN-α (0, 1, 10, 100 IU/ml) to the 1B-4RL8 and KAH5RL8 cells (each cultured for 2 days after inoculating 2×10⁴ cells in a 24-well plate). Measurements were made in at least 3 wells at each point. SD values are also shown in the figure. A one hundred percent measurement value of 300,000 was obtained for the 1B-4RL8 and KAH5RL8 cells. Though the result is slightly below the results obtained for ORL8 and ORL11 cells (FIG. 14), the value is sufficient for the analysis of drug anti-HCV effects. The graphs on the right represent the results of quantitative HCV RNA measurements by LightCycler PCR 24 hours after the addition of IFN-α (0, 1, 10, 100 IU/ml) to the 1B-4RL8 and KAH5RL8 cells (each cultured for 2 days after inoculating 2×10⁵ cells in a 6-well plate). Measurements were made in at least 3 wells at each point. SD values are also shown in the figure. As represented in the figure, the luciferase activity and the HCV RNA level decreased in a manner that depended on IFN-α concentration.

The results of these measurements had a good correlation as did the results from OR6 cells. It was therefore demonstrated that the 1B-4RL8 and KAH5RL8 cells were useful for the quantification of HCV RNA replication level with a simple luciferase assay, and could provide desirable assay systems for the activity evaluation of anti-HCV agents against 1B-4 strain HCV and KAH5 strain HCV. Specifically, it can be said that ORL8c cells have not only infectious JFH1 strain HCV production capability, but the ability to permit replication of HCV RNAs of new HCV strains (particularly, replication of RNAs longer than the original HCV RNA (9.6 kb), as in the 12-kb RNA having a luciferase gene).

The present invention is not limited to the description of the embodiments above, but may be altered within the scope of the claims. An embodiment based on a proper combination of technical means disclosed in different embodiments is encompassed in the technical scope of the present invention.

All academic papers and patent documents cited in this specification are incorporated herein by reference.

### INDUSTRIAL APPLICABILITY

According to the present invention, an HCV life cycle reproduction system derived from a non-HuH-7 cell line can be constructed. Further, combined use of this system with an HCV life cycle reproduction system derived from a known HuH-7 cell line enables the detection of a substance that is known to have an anti-HCV action, and also enables the detection of the anti-HCV action of a substance that is considered not to have an anti-HCV action. Accordingly, the present invention can provide a method of screening a substance having an anti-HCV action, and a therapeutic agent for hepatitis C, and thus contributes to the development of the reagent industry, the pharmaceutical industry, etc.

1. A method of producing an HCV replicon-replicating cell, comprising
a step of introducing RNA containing an HCV replicon sequence and a selectable marker gene sequence into a Li23 cell or a cured cell derived from a Li23 cell,
wherein the HCV replicon sequence comprises a base sequence encoding an amino acid sequence as set forth in SEQ ID NO: 2 containing amino acid substitutions at Q1112R, K1609E and S2200R or at Q1112R, P1115L and S2200R.

2. The method according to claim 1 wherein the HCV replicon sequence is as set forth in SEQ ID NO: 3 or 5.

3. A method of producing a full-length HCV RNA-replicating cell, comprising a step of introducing RNA containing a full-length HCV genome sequence and a selective marker into a cured cell derived from a Li23 cell.

4. The method according to claim 3 wherein the full-length HCV genome sequence comprises a base sequence encoding an amino acid sequence as set forth in SEQ ID NO: 2 containing amino acid substitutions at Q1112R, K1609E and S2200R or at Q1112R, P1115L and S2200R.

5. The method according to claim 4 wherein the base sequence is as set forth in SEQ ID NO: 7 or 9.

6. The method according to claim 4 wherein the full-length HCV genome sequence contains a base sequence as set forth in SEQ ID NO: 11 or 13.

7. The method according to claim 3 wherein the RNA further contains a reporter gene sequence.

8. The method according to claim 3 wherein the RNA further contains an exogenous internal ribosomal entry site (IRES) sequence.

9. A method of screening a substance having an anti-HCV action, comprising
a step of incubating a cell prepared by the method of claim 1 with a candidate agent; and
a step of measuring the level of an HCV gene product.

10. A kit for screening a substance having an anti-HCV action, comprising
a cell prepared by the method of claim 1; and
a reagent for measuring the level of an HCV gene product.

11. A method of screening a substance having an anti-HCV action, comprising
a step of incubating a cell prepared by the method of claim 7 with a candidate agent; and
a step of measuring the level of a reporter gene product.

12. A kit for screening a substance having an anti-HCV action, comprising
a cell prepared by the method of claim 7; and
a reagent for measuring the level of a reporter gene product.

13. A method of producing a cured cell derived from a Li23 cell, comprising a step of culturing the cell prepared by the method of claim 1 in a medium containing a pharmaceutical agent having an anti-viral action.

14. A method of producing an infectious HCV particle, comprising a step of incubating the cured cell prepared by the method of claim 13 with infectious HCV RNA.
